(19) United States
(12) Patent Application Publication
(10) Pub. No.: US 2025/0361469 A1
(43) Pub. Date: Nov. 27, 2025

(54) Lymph Node on a Chip Device and Method

(71) Applicant: New York University, New York, NY (US)

(72) Inventors: Weiqiang Chen, New York, NY (US); Fanghao Shi, Brooklyn, NY (US)

(21) Appl. No.: 19/214,577

(22) Filed: May 21, 2025

Related U.S. Application Data
(60) Provisional application No. 63/651,081, filed on May 23, 2024.

(51) Int. Cl. C12M 3/00; C12M 1/00; C12M 3/06; G01N 33/50

(52) U.S. Cl. CPC C12M 21/08; C12M 23/02; C12M 23/16; C12M 23/34; C12M 23/40; G01N 33/5088

(57) ABSTRACT

A lymph node on a chip device includes a microfluidic chip having a top and bottom surface, a central chamber embedded in the chip, one or more openings in the chip fluidly connected to the central chamber with one or more channels, a plurality of micropillars arranged within the central chamber such that the central chamber is partitioned into an inner region, one or more outer regions positioned around the inner region, and a circumferential region surrounding the one or more outer regions, with the micropillars forming channels extending from the inner region to at least the outer region, and paracortex cells configured to mimic a paracortex region positioned in the inner region, follicle cells configured to mimic one or more follicle regions positioned in the one or more outer regions, and interfollicular cells configured to mimic one or more interfollicular regions positioned in the one or more channels.

### CROSS-REFERENCE TO RELATED APPLICATIONS

This application claims priority to U.S. Provisional Application No. 63/651,081 filed on May 23, 2024, incorporated herein by reference in its entirety.

### STATEMENT REGARDING FEDERALLY SPONSORED RESEARCH OR DEVELOPMENT

This invention was made with government support under R35 GM133646 awarded by the National Institutes of Health. The government has certain rights in the invention.

### BACKGROUND OF THE INVENTION

Vaccines play an essential role in controlling and preventing infectious diseases in humans [Dube et al., Annu Rev Public Health 42, 175-191 (2021); M. Jeyanathan et al., Nat Rev Immunol 20, 615-632 (2020); Pingali et al., Morbidity and Mortality Weekly Report 70, 1183-1190 (2021)]. Successful vaccination requires efficient delivery of antigenic substances to induce a strong therapeutic or protective immune response [S. Sell, Expert Rev Vaccines 18, 993-1015 (2019); L. Sompayrac, How the Immune System Works. (2019)]. Because vaccine antigens primarily activate the adaptive immune system, which remembers and eliminates the antigen as a foreign invader, the efficacy of the vaccine depends on the extent to which the vaccine enhances the adaptive immune response in the body [Ding et al., Adv Drug Deliv Rev 179, 113914 (2021); Leleux et al., J Control Release 219, 610-621 (2015)]. Adaptive immune response is mainly initiated in lymph nodes (LNs), which serve as vital hubs for adaptive immune-related lymphocytes, including antigen-presenting cells (APCs), CD4+ helper T cells, CD8+ T cells, and B cells [Grant et al., J Cell Sci 133, (2020)]. However, depletion of CD4+ T cells, reduced germinal centers, and LN extracellular matrix (ECM) fibrosis due to aging and chronic disease can lead to delayed immune responses and hinder the generation of memory B cells, affecting vaccine efficacy across populations [Cakala-Jakimowicz et al., Cells 10, (2021); Chen et al., Trends Mol Med 28, 1100-1111 (2022); Ahmadi et al., ANZ J Surg 83, 612-618 (2013)]. Therefore, understanding the impact of the LN niche on adaptive immunity is critical for developing effective vaccines and new vaccination strategies tailored to different populations.

Vaccine development is an exceedingly intricate and time-consuming process [B. C. Buckland, Nature Medicine 11, S16-S19 (2005)]. The lack of comprehensive preclinical data, and a dearth of precise information on the correlates of immune protection have frequently led to vaccine products failing in clinical trials [Pulendran & Davis, Science (New York, N.Y.) 369, (2020)]. To address this issue, it is crucial to develop more relevant animal models and to collect and analyze human samples extensively.

However, interspecies differences between animal models and humans hinder accurate replication of immune responses following vaccination [Walls et al., Cell Rep 40, 111299 (2022)]. Furthermore, ethical and safety concerns surrounding paid recruitment of clinical volunteers have faced widespread criticism [Calina et al., Journal of Faculty of Pharmacy, Tehran University of Medical Sciences 28, 807-812 (2020)]. These underscore the pressing importance of devising new vaccine testing models.

LNs, as secondary immune organs, play a pivotal role in capturing vaccine antigens and generating both memory cells for long-term immunity and plasma cells for antibody secretion [Moysi et al., Expert Rev Vaccines 21, 633-644 (2022)]. The adaptive immune processes that occur in LNs after vaccination form the foundation for the body's enduring immunity [Amanna & Slifka, Curr Top Microbiol Immunol 428, 1-30 (2020)].

Thus, there is the need in the art for humanized models that simulate adaptive responses within human LNs and investigate the variations in vaccine responses among different populations. The present invention meets this need.

### SUMMARY OF THE INVENTION

Aspects of the present invention relate to a lymph node on a chip device including a microfluidic chip having a top and bottom surface, a central chamber embedded in the microfluidic chip, one or more openings in the chip fluidly connected to the central chamber with one or more channels, a plurality of micropillars arranged within the central chamber such that the central chamber is partitioned into an inner region, one or more outer regions positioned around the inner region, and a circumferential region surrounding the one or more outer regions, with the micropillars forming one or more channels extending from the inner region to at least the outer region, and one or more paracortex cells configured to mimic a paracortex region positioned in the inner region, one or more follicle cells configured to mimic one or more follicle regions positioned in the one or more outer regions, and one or more interfollicular cells configured to mimic one or more interfollicular regions positioned in the one or more channels.

In some embodiments, the one or more paracortex cells are selected from the group consisting of: paracortex niche cells, paracortex niche supporting cells, stromal cells, T cells, T lymphocytes, T helper cells, cytotoxic T cells, regulatory T cells (Tregs), dendritic cells (DC), fibroblastic reticular cells (FRC), and blood vessel endothelial cells.

In some embodiments, the one or more follicle cells are selected from the group consisting of: follicle niche cells, follicle niche supporting cells, B cells, naïve B cells, memory B cells, plasma cells, follicular dendritic cells (FDC), and blood-derived dendritic cells (DC).

In some embodiments, the one or more interfollicular cells are selected from the group consisting of: interfollicular niche cells, interfollicular niche supporting cells, stromal cells, fibroblastic reticular cells (FRC), endothelial cells, blood vessel cells, blood vessel endothelial cells, T cells, T lymphocytes, T helper cells, cytotoxic T cells, and regulatory T cells (Tregs)

In some embodiments, the device further includes one or more subcapsular sinus cells configured to mimic a subcapsular sinus positioned in the circumferential region.

In some embodiments, the one or more subcapsular sinus cells are selected from the group consisting of: sinus cells, subcapsular sinus cells, macrophages, lymphatic endothelial cells, marginal reticular cells, dendritic cells.

In some embodiments, the device further includes one or more cell culture media components in the central chamber selected from the group consisting of: cell culture media, growth factors, growth factors for fibroblasts, growth factors for endothelial cells, fibroblast medium (2301, ScienCell), RPMI 1640 medium (Gibco), endothelial cell growth medium (EGM-2, Lonza), and Vascular endothelial growth factor (VEGF).

In some embodiments, the device further includes one or more extracellular matrix components in the central chamber selected from the group consisting of: membrane, basement membrane, solubilized basement membrane, Matrigel (Corning), polymer, gel, hydrogel, fibrin hydrogel (Sigma), collagen, collagen I (Corning).

In some embodiments, the device further includes one or more reservoirs embedded in the microfluidic chip fluidly connected to the central chamber.

In some embodiments, the central chamber is at least partially formed in one or more shapes selected from the group consisting of: irregular, limagon, cardioid, heart, kidney, elliptical, ovular, and round.

In some embodiments, the central chamber is formed in a cardioid shape and the one or more reservoirs include a first reservoir fluidly connected to the cusp region of the central chamber, and a second and third reservoirs fluidly connected to positions opposite the cusp region of the central chamber.

In some embodiments, each micropillar of the plurality of micropillars is at least partially formed in one or more shapes selected from the group consisting of: column, cylinder, round, frustum, cone, oblong, irregular.

In some embodiments, each micropillar has a width or diameter, a height, and a spacing to the next micropillar; wherein the width or diameter ranges between about 100 μm and about 200 μm, the height ranges between about 50 μm and about 200 μm, and the spacing to the next micropillar ranges between about 50 μm and about 200 μm.

Aspects of the present invention relate a method of measuring an immune response having the steps of providing a lymph node on a chip device (e.g., device 100 disclosed herein), administering at least one treatment to the device, and determining treatment responsiveness based on at least one measured change on the device.

In some embodiments, the at least one treatment is selected from the group consisting of: vaccine, mRNA vaccine, inactivated vaccine, adenovirus-based vaccine, small molecule, protein, and nucleic acid molecule.

In some embodiments, the measured change includes any of: antibody secretions, cell migration, cell proliferation, cell activation, cell infiltration, cell speed, cell trajectory, cell distance, cell position, cell motility, cell maturation, cell maturation efficiency, cell maturation efficiency, cell phenotype, cell differentiation, cell concentration, cell recruitment, cell migration within one region (e.g., the inner region), cell migration from one region to another (e.g., the inner region to the interfollicular region), pH in the central chamber, pH in a culture medium, number, size and spatial distribution of germinal centers, number, size and spatial distribution of germinal centers in the one or more outer regions.

In some embodiments, the at least one measured change includes antibody secretion (e.g., immunoglobulin such as IgG, IgM, IgE, IgD, or secretion levels of total IgG, secretion levels of total IgM), and T cell migration from the paracortex region to the interfollicular region.

In some embodiments, the at least one measured change further includes cytokine concentration.

In some embodiments, the device further includes the step of administering at least one adjuvant in combination with the at least one treatment. In some embodiments, the at least one adjuvant is selected from the group consisting of: AS03, CpG Oligodeoxynucleotides, squalene, monophosphoryl Lipid A, aluminum salts and MF59, or any combinations thereof.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A depicts an exemplary lymph node (LN) on a chip device according to aspects of the present invention.

FIG. 1B depicts an enlarged view of a central chamber of the LN on a chip device of FIG. 1A.

FIG. 1C depicts an exemplary central chamber of an LN on a chip device according to aspects of the present invention.

FIG. 1D depicts an exemplary central chamber (left) and an exemplary LN on a chip device (right) according to aspects of the present invention.

FIG. 1E is a diagram depicting a method of measuring an immune response using the disclosed LN on a chip device according to aspects of the present invention.

FIG. 2A is an image depicting the structure and function of human LNs.

FIG. 2B is a diagram showing the adaptive immune process in LN after vaccination.

FIG. 3A depicts an exemplary central chamber of an LN on a chip device with a human LN in vitro model comprising an organotypic microfluidic chip design according to aspects of the present invention.

FIG. 3B depicts various adaptive immunity research techniques (e.g., adaptive immune response research) enabled by the disclosed LN-on-the-chip device.

FIG. 3C are images of a whole scan of the LN-on-a-chip device.

FIG. 3D is an image showing blood vessels in the paracortex region of the disclosed LN-on-a-chip device.

FIG. 4A through FIG. 4D is a diagram depicting LN organization. FIG. 4A shows that dendritic cells (DCs) arrive at the LN via afferent vessels and then migrate into the cortex. FIG. 4B shows that B cells are located in follicles and interact with follicular dendritic cells (FDCs). FIG. 4C shows that T cells are in the paracortex to interact with DCs, supported by fibroblastic reticular cells (FRCs). FIG. 4D shows that DCs migrate on reticular fibers to the high endothelial venules (HEVs), where they interact with naïve lymphocytes entering the LN from the HEV, and activated B and T cells crawl along the medullary sinus to leave the LN.

FIG. 5A through FIG. 5F shows remodeling of the stromal niche of paracortical regions on the disclosed LN on a chip device. FIG. 5A is images showing HEVs and FRCs reticular system in the paracortical areas of the disclosed LN on a chip device. FIG. 5B shows perivascular FRC around blood vessels that support vessel integrity on the disclosed LN on a chip device. FIG. 5C shows T cells attach and grow on the FRC surface. FIG. 5D and FIG. 5E are plots showing results of the secretion of CCL19 (FIG. 5D) and CCL21 (FIG. 5E) by DCs and FRCs in the paracortical area increases under TNF-α. FIG. 5F is images showing T cells migrate along the FRCs stromal reticulum on the disclosed LN on a chip device.

FIG. 6A through FIG. 6D depict a DC-mediated T cell activation on the disclosed LN on a chip device. FIG. 6A depicts the maturation of DCs. FIG. 6B shows imaging and results of the effects of OVA I-induced mDCs and imDCs (control) on T cell migration in the paracortex area on disclosed LN on a chip device. FIG. 6C is a plot showing results indicating that T cell proliferation is higher on chips with mature DCs (mDCs) than chips with immature DCs (imDCs). FIG. 6D is plots showing results indicating the secretion of T cell activation-related cytokines increased in chips with mDCs than chips with imDCs.

FIG. 7A through FIG. 7C show B cell differentiation and germinal center formation on the disclosed LN on a chip device. FIG. 7A is a diagram showing a paracortex region and formation of germinal center structure. FIG. 7B is a staining image of germinal center structure formed in B cell follicles on the chip device upon antigen stimulation. FIG. 7C are staining images and quantitative results showing CD138+ plasma cells in B cell follicles with (left) and without (right) antigen stimulation.

FIG. 8 is a set of images (left) and plot (right) showing results of AID expression of B cells derived from peripheral blood in the disclosed LN on a chip device.

FIG. 9A through FIG. 9C show plots of results for the evaluation of seasonal influenza virus vaccine on the disclosed LN on a chip device using cells sampled from different subject age groups. FIG. 9A is a plot showing the results of the changes in key cytokines/chemokines of adaptive immunity. FIG. 9B is a plot showing the results of cell differentiated CD138+ plasma cells. FIG. 9C is a plot showing the results of Total Immunoglobulin G (IgG) secretion.

FIG. 10A and FIG. 10B depict an exemplary human LN-on-a-Chip device in vitro model and imaging scans of the device. FIG. 10A is a diagram depicting an exemplary central region (left) and microfluidic chip design (right) for a human LN-on-a-Chip device. FIG. 10B is a set of images showing a whole scan of cells on the chip (left) and enlarged views of specific regions of the chip (right top, right bottom).

FIG. 11A and FIG. 11B depict exemplary configurations for the disclosed LN on a chip device and the related remodeling of stromal cell distribution within the chip through sustained lymph fluid perfusion. FIG. 11A depicts a static culture (left, middle) and shows the results for the static culture (right). FIG. 11B depicts a dynamic culture (left, middle) and shows the results for the dynamic culture (right).

### DETAILED DESCRIPTION

The present invention relates to devices that mimic lymph nodes (LNs) and/or LN microenvironments in a microfluidic chip, and associated methods of use. The devices can be used to model response and/or effectiveness of therapy, or in other examples, to model certain disease states related to the LNs, such as lymphatic diseases. The devices can be adapted to replicate the microenvironment from patient-specific cells such that treatment conditions can be modeled and tailored to individual patients. In some embodiments, the devices are suitable for evaluating any therapy including, but not limited to, vaccine, mRNA vaccine, inactivated vaccine, adenovirus-based vaccine, small molecule, protein, nucleic acid molecule, anti-cancer drug, immunotherapy, chemotherapy, radiation therapy, chemoradiation therapy, and targeted therapy on a patient-specific basis.

It is to be understood that the figures and descriptions of the present invention have been simplified to illustrate elements that are relevant for a clear understanding of the present invention, while eliminating, for the purpose of clarity many other elements found in related devices, systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Lymph Node-on-a-Chip Device

The disclosed LN on a Chip device (in some examples referred to as an “LN on a Chip in vitro model”, or “LN chip”) recapitulates human LN function and structural compartmentalization, vasculature, stromal and immune niches, while providing biological and biophysical controls over the device. In some embodiments, the disclosed LN on a Chip device comprises a 3D organotypic human LN model using state-of-the-art microfluidic organ-on-a-chip technology to create a physiologically relevant LN microenvironment. The disclosed device mimics natural LN niche compartments (paracortex, follicle, subcapsular sinus) with extracellular matrix (ECM), T cells, B cells, dendritic cells (DCs), fibroblasts, lymphatic vessels, and continuous lymph fluid perfusion. In some embodiments, the disclosed LN-on-a-Chip device is designed for human adaptive immunity modeling and vaccine or therapy evaluation. In some embodiments, the device can recapitulate adaptive immune environments of varied populations in vitro to monitor crucial immune responses including, but not limited to, cell chemotaxis, lymphocyte activation, and antibody secretion.

The disclosed LN on a Chip device provides a more accurate representation of adaptive immune responses than traditional animal models for vaccine efficacy evaluation. In some embodiments, the disclosed device allows testing of vaccines and adjuvants on long-term adaptive immune responses from various populations, considering factors like age, gender, race, health status, and genetic variations, thus aiding in optimizing vaccination strategies for optimal protection for different populations.

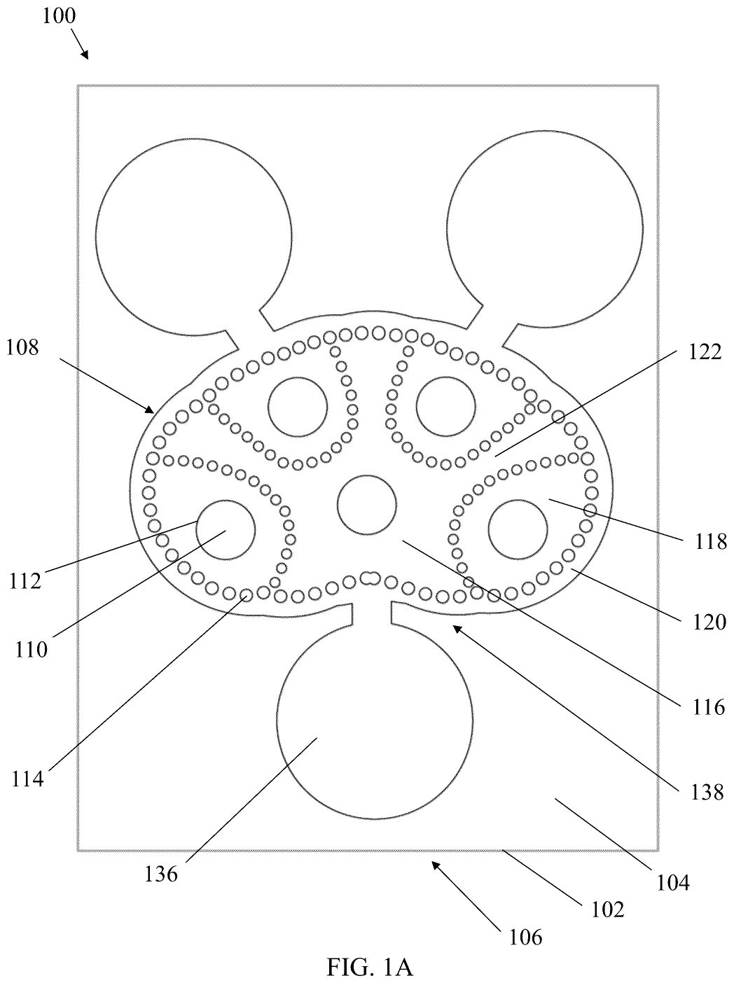

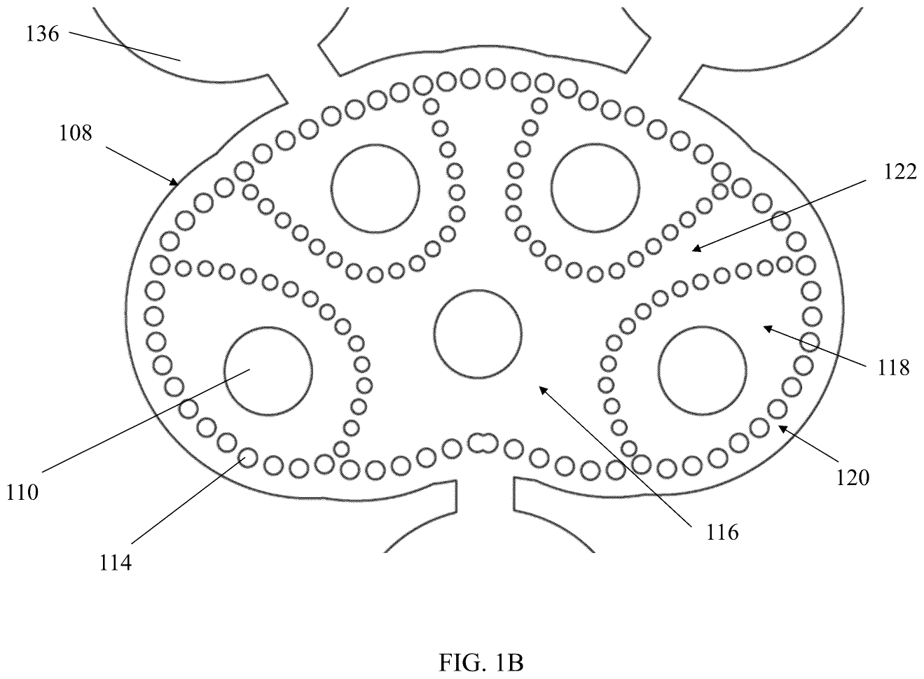

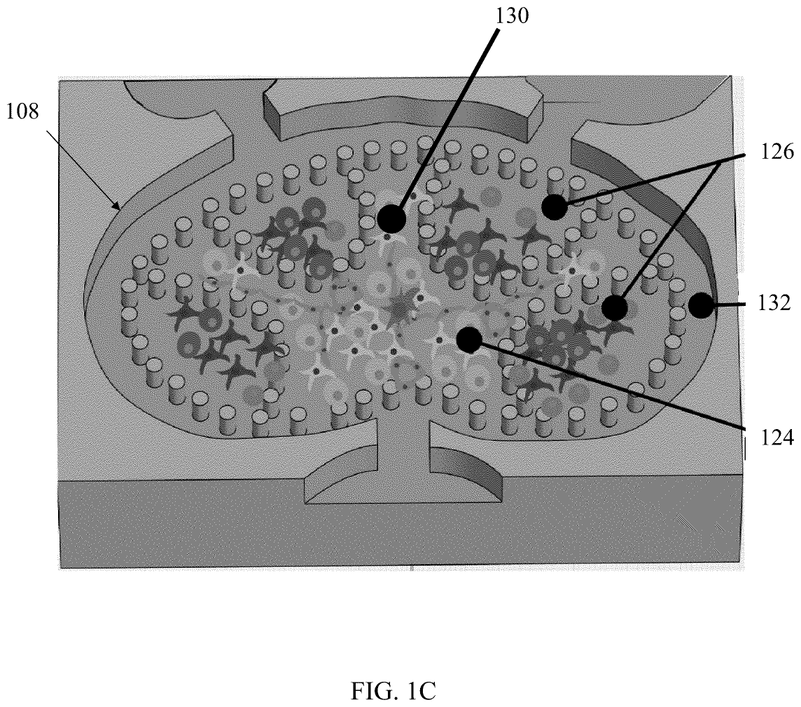

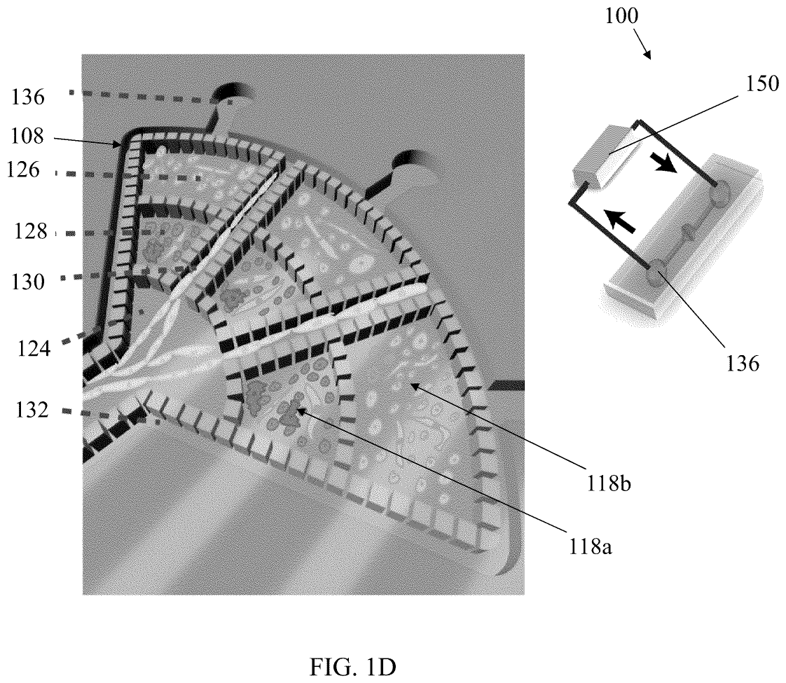

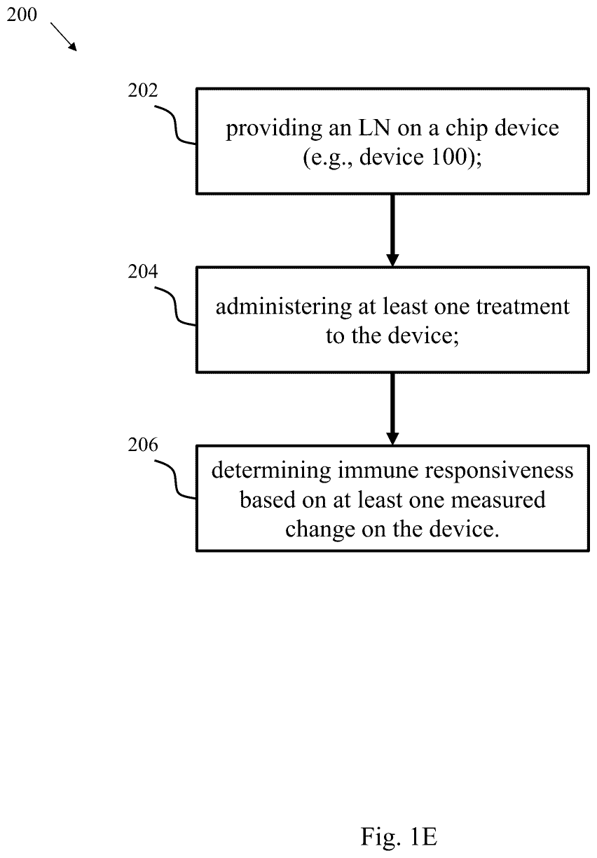

Aspects of the present invention relate to an LN on a Chip device comprising a microfluidic chip configured to mimic or recapitulate at least one lymph node. Referring now to FIG. 1A, shown is an exemplary LN on a chip device 100 according to aspects of the present invention. In some embodiments, device 100 comprises a microfluidic chip 102 comprising a top surface 104 and bottom surface 106, at least one central chamber 108 embedded in the microfluidic chip 102, and one or more openings 110 in the microfluidic chip 102 fluidly connected to the central chamber 108 with one or more channels 112. Device 100 and configurations thereof may be configured on, connected to, or constructed with, any known microfluidic chip, device, or system, organ on chip device or system, and the like. Further, device 100 may comprise any known microfluidic chip components, pumps, sensors, fluids and supporting equipment as would be known by one of normal skill in the art.

Device 100 generally comprises a microfluidic chip 102 with at least one central chamber 108 containing a plurality of micropillars 114 arranged to create one or more areas or regions to recapitulate at least one LN niche or microenvironment. Referring now to FIG. 1B, in some embodiments, device 100 comprises a central chamber 108 comprising a plurality of micropillars 114 configured in an arrangement intended to mimic portions of at least one LN niche or microenvironment. The plurality of micropillars 114 serve to partition the central chamber 108 into distinct, but fluidly connected regions or areas. For example, in some embodiments, device 100 comprises a plurality of micropillars 114 arranged within the central chamber 108, such that the central chamber 108 is partitioned into an inner region 116, one or more outer regions 118 positioned around the inner region 116, and a circumferential region 120 surrounding the one or more outer regions 118. In some embodiments, central chamber 108 comprises one or more channels 122 extending from at least the inner region 116 to the one or more outer regions 118, and/or to the circumferential region 120. In some embodiments, the inner region 116 is intended to mimic the paracortex region of an LN, the one or more outer regions 118 mimic at least one follicle region, the circumferential region 120 mimics a subcapsular sinus region, and the one or more channels 122 mimic an interfollicular region. In some embodiments, inner region 116 and one or more channels comprise one continuous region or area within central chamber 108. In some embodiments, device 100 further comprises one or more reservoirs 136 fluidly connected to the central chamber 108. In some embodiments, the one or more reservoirs 136 are embedded in the microfluidic chip 102.

Aspects of the present invention relate to positioning one or more cells in central chamber 108 within specific areas or regions in order to recapitulate an LN niche or microenvironment on device 100. Referring now to FIG. 1C, in some embodiments, device 100 comprises one or more paracortex cells 124 configured to mimic a paracortex region positioned in the inner region 116, one or more follicle cells 126 configured to mimic one or more follicle regions positioned in the one or more outer regions 118, and one or more interfollicular cells 130 configured to mimic one or more interfollicular regions positioned in the one or more channels 122. In some embodiments, device 100 comprises one or more subcapsular sinus cells 132 configured to mimic a subcapsular sinus positioned in the circumferential region 120.

Aspects of the present invention relate to providing one or more paracortex cells to central chamber 108 in order to recapitulate a paracortex region on device 100. In some embodiments, the one or more paracortex cells 124 are loaded or positioned into inner region 116 of central chamber 108. In some embodiments, the one or more paracortex cells 124 are selected from the group consisting of: paracortex niche cells, paracortex niche supporting cells, stromal cells, T cells, T lymphocytes, T helper cells, cytotoxic T cells, regulatory T cells (Tregs), dendritic cells (DC), fibroblastic reticular cells (FRC), and blood vessel endothelial cells.

Aspects of the present invention relate to providing one or more follicle cells to central chamber 108 in order to recapitulate a follicle region on device 100. For example, in some embodiments, one or more follicle cells 126 are loaded or positioned into outer region 118 of central chamber 108. In some embodiments, the one or more follicle cells 126 are selected from the group consisting of: follicle niche cells, follicle niche supporting cells, B cells, naïve B cells, memory B cells, plasma cells, follicular dendritic cells (FDC), and blood-derived dendritic cells (DC).

Aspects of the present invention relate to providing one or more interfollicular cells to central chamber 108 in order to recapitulate an interfollicular region on device 100. In some embodiments, the one or more interfollicular cells 130 are loaded or positioned in the one or more channels 122 of central chamber 108. In some embodiments, the one or more interfollicular cells 130 are selected from the group consisting of: interfollicular niche cells, interfollicular niche supporting cells, stromal cells, fibroblastic reticular cells (FRC), endothelial cells, blood vessel cells, blood vessel endothelial cells, T cells, T lymphocytes, T helper cells, cytotoxic T cells, and regulatory T cells (Tregs).

Aspects of the present invention relate to providing one or more subcapsular sinus cells to central chamber 108 in order to recapitulate a subcapsular sinus region on device 100. For example, in some embodiments, one or more subcapsular sinus cells 132 are positioned in the circumferential region 120 of central chamber 108. In some embodiments, the one or more subcapsular sinus cells 132 are selected from the group consisting of: sinus cells, subcapsular sinus cells, macrophages, lymphatic endothelial cells, marginal reticular cells, dendritic cells.

Referring now to FIG. 1D, in some embodiments, the one or more outer regions 118 comprise at least a first outer region 118a and a second outer region 118b. In some embodiments, first outer region 118a comprises one or more paracortex cells, or paracortex supporting cells, and first outer region 118b comprises one or more follicle cells 126. In some embodiments, the first outer region 118a comprises one or more paracortex cells 128, separate from the paracortex cells 124 of inner region 116. In some embodiments, the one or more paracortex cells 128 are selected from the group consisting of: paracortex niche cells, paracortex niche supporting cells, stromal cells, T cells, T lymphocytes, T helper cells, cytotoxic T cells, regulatory T cells (Tregs), dendritic cells (DC), fibroblastic reticular cells (FRC).

Aspects of the present invention relate to providing or perfusing one or more fluids to or through at least a portion of device 100 (e.g., reservoir 136, central chamber 108). Device 100 may be configured with a static configuration, only utilizing reservoirs embedded in the microfluidic chip 102, or in a dynamic or flow configuration, wherein at least one pump or perfusion system provides fluid flow to microfluidic chip 102. In some embodiments, device 100 further comprises one or more pumps 150 fluidly connected to central chamber 108. In some embodiments, the one or more pumps 150 fluidly connect to the one or more reservoirs 136. In some embodiments, one or more fluids may be loaded or filled into the one or more reservoirs 136 in order to fluidly supply or perfuse central chamber 108. In some embodiments, a flow rate is provided to at least a portion of device 100 ranging between from 0-120 μL/h to simulate lymphatic flow within the device. For example, in some embodiments, a fluid flow of about 60 μL/h is provided to one or more reservoirs 136 and central chamber 108. It should be appreciated that device 100 may comprise any known tubes, conduits, connectors, manifolds, pumps, fluid sources, controllers, or combinations thereof, as would known by one of normal skill in the art in order to provide a desired flow of fluid to or from the device.

Aspects of the present invention relate to supporting or culturing one or more cells on device 100 to recapitulate an LN niche. For example, in some embodiments, one or more cell media components are provided to at least a portion of device 100 (e.g., central chamber 108 and/or reservoir 136). In some embodiments, the one or more cell culture media components are selected from the group consisting of: cell culture media, growth factors, growth factors for fibroblasts, growth factors for endothelial cells, fibroblast medium (2301, ScienCell), RPMI 1640 medium (Gibco), endothelial cell growth medium (EGM-2, Lonza), and Vascular endothelial growth factor (VEGF). In some embodiments, device 100 further comprises one or more ECM components positioned within or on at least a portion of device 100 (e.g., central chamber 108 and/or reservoir 136) selected from the group consisting of: membrane, basement membrane, solubilized basement membrane, Matrigel (Corning), polymer, gel, hydrogel, fibrin hydrogel (Sigma), collagen, collagen I (Corning), and any combinations thereof.

Aspects of the present invention relate to forming a central chamber 108 in device 100 that recapitulates an LN niche. For example, in some embodiments, central chamber 108 is at least partially shaped like a typical human LN. In some embodiments, the central chamber 108 is at least partially formed in one or more shapes selected from the group consisting of: irregular, limagon, cardioid, heart, kidney, elliptical, ovular, round, and any combinations thereof. In some embodiments, the central chamber 108 is formed in a cardioid shape and the one or more reservoirs 136 comprise a first reservoir fluidly connected to a cusp region 138 of the central chamber 108, and a second reservoir and a third reservoir is fluidly connected to positions opposite the cusp region 138 of the central chamber 108. In some embodiments, the one or more reservoirs 136 comprises 1 reservoir, 2 reservoirs, 3 reservoirs, 4 reservoirs, 5 reservoirs, 6 reservoirs, 7 reservoirs, 8 reservoirs, 9 reservoirs, 10 reservoirs, or any number of reservoirs. For example, in some embodiments, the one or more reservoirs 136 comprises 5 reservoirs positioned around central chamber 108 on microfluidic chip 102. In some embodiments, device 100 comprises more than one central chamber 108, each central chamber 108 fluidly connected to at least one other central chamber 108. For example, in some embodiments, device 100 comprises 1, 2, 3, 4, 5, 6 7, 8 9, or 10 central chamber 108, or a plurality of central chambers 108.

Aspects of the present invention relate to sizes and dimensions of device 100. In some embodiments, microfluidic chip 102 has a length ranging between 1 mm and 100 mm, a width ranging between 1 mm and 100 mm, and a height ranging between 1 mm and 100 mm. For example, in some embodiments, microfluidic chip 102 has a length of about 15 mm, a width of about 10 mm, and a height of about 5 mm. In some embodiments, central chamber 108 has a width or diameter ranging between 1 mm and 100 mm, and length ranging between 1 mm and 100 mm, and a height or depth ranging between 0.01 mm and 50 mm. For example, in some embodiments, central chamber 108 has a diameter of about 4 mm, and a depth of about 0.08 mm. In some embodiments, each reservoir 136 has a diameter ranging between 1 mm and 50 mm, and a height or depth ranging between 0.01 mm and 50 mm.

In some embodiments, inner region 116 has an area ranging between 0.1 mm² and 100 mm², one or more outer regions 118 each have an area ranging between 0.1 mm² and 20 mm², circumferential region 120 has an area ranging between 0.1 mm² and 10 mm². In some embodiments, one or more channels 122 have an area ranging between 0.1 mm² and 10 mm².

Aspects of the present invention relate to the size and shapes of the micropillars of device 100. For example, in some embodiments, each micropillar of the plurality of micropillars 114 is at least partially formed in one or more shapes selected from the group consisting of: column, cylinder, round, frustum, cone, oblong, irregular. In some embodiments, each micropillar of the plurality of micropillars 114 has a width or diameter, a height, and a spacing to the next micropillar; wherein the width or diameter ranges between about 100 μm and about 200 μm, the height ranges between about 50 μm and about 200 μm, and the spacing to the next micropillar ranges between about 50 μm and about 200 μm. In some embodiments, each micropillar has the same shape, width or diameter, height, and spacing, or may have different shape, width or diameter, height and spacing.

In some embodiments, device 100 provides a platform for multidimensional evaluation of vaccine effectiveness. In some embodiments, device 100 enables testing the effects of different vaccines (e.g. human seasonal influenza virus vaccine, rabies virus vaccine) and adjuvants (e.g., MF59, AS03) in LNs.

In some embodiments, device 100 may be configured to replicate one or more disease states (e.g., lymphoma). In some embodiments, device 100 effectively replicates the complex microenvironment of LNs, critical sites for the development and evolution of lymphoma. Importantly, the configuration of device 100 does not require structural changes in order to replicate one or more disease states. The device can be used to simulate B-cell lymphoma simply by changing the cell types, such as introducing B-cell lymphoma cells. By simulating the physiological conditions of LNs, device 100 enables researchers to delve deeper into the mechanisms of lymphoma's initiation, growth, and spread within a highly realistic lymphatic context. The capabilities of device 100 are instrumental in elucidating the complex interactions between lymphoma cells and the immune system. Understanding these dynamics is essential for the advancement of novel immunotherapeutic strategies, which aim to harness and enhance the body's immune response against cancer cells. The device's ability to mimic these interactions can reveal new pathways and targets for intervention, potentially leading to breakthroughs in cancer immunotherapy. In some embodiments, device 100 can be personalized for the individual or subject. By incorporating cells derived from individual lymphoma patients, device 100 can simulate the specific characteristics of a patient's cancer, paving the way for personalized medicine approaches. This individualized strategy can revolutionize treatment protocols, allowing clinicians to tailor therapies based on the unique genetic and molecular profile of each patient's lymphoma, thus maximizing efficacy and minimizing side effects.

In some embodiments, device 100 may be configured to replicate one or more disease states, such as LN metastasis. Within a subject, cancer cells break away from the primary tumor, travel through the lymphatic system, and establish new tumors in LNs. The presence of tumor cells in the LN is often a critical factor in cancer staging, which helps determine the prognosis and treatment strategy. In some embodiments, device 100 can be combined with other tumor chips to study in detail how cancer cells metastasize to lymph nodes and the subsequent immune response. This is particularly significant in understanding and combating cancer spread. Furthermore, in some embodiments, device 100 may be used for specific antigen screening. By identifying unique proteins or antigens present on tumor cells, it aids in understanding the immune system's recognition and response mechanisms, which are central to developing targeted immunotherapies like cancer vaccines or adoptive T cell therapies. Additionally, the adaptability of device 100 to use patient-specific cells marks a significant stride towards personalized medicine, enabling the modeling of individual tumor-immune interactions and facilitating the testing of tailored treatment strategies. This personalized approach is instrumental in determining the most effective antigens and therapies for each patient.

In some embodiments, device 100 comprises key LN niche factors in central chamber 108 with controllable biological features (e.g. cell type and density, extra-cellular matrix (ECM) components, biophysical parameters like oxygen level and IFP). In some embodiments, device 100 recapitulates an LN with patient-specific organoids and/or patent derived organoids (PDOs). In some embodiments, device 100 maintains the original patient-specific features in PDOs with high pathological relevance. In some embodiments, device 100 enables real-time characterization of pathophysiological processes at high spatiotemporal resolution.

### Fabrication of Device

Aspects of the present invention relate to a method of fabricating an LN on a chip device (e.g., device 100). In some embodiments, microfluidic chip 102 of device 100 comprises a PDMS-based microfluidic chip fabricated using a standard soft lithography replica-molding method [Ma et al., Sci Adv. 2020; 6(44)]. In some embodiments, the different culture compartments (e.g., inner region 116, one or more outer regions 118, circumferential region 120, and one or more channels 122) are partitioned by regularly spaced PDMS micropillars that confine cell-embedded hydrogels. In some embodiments, T cells, and FRCs are loaded into the paracortex area (i.e., inner region 116, also referred to herein as the “T cell zone”), and B cells and FDCs were loaded into the B cell follicle region (i.e., one or more outer regions 118) in device 100. Additionally, in some embodiments, 3D blood vessels are created mimicking high endothelial venule (HEV) located in the T cell zone of the LN chip for lymphocytes trafficking, by compartmentalizing human primary endothelial cells (HUVECs) in the paracortex and surrounding channels (i.e., one or more channels 122) of the immune compartments (e.g., central chamber 108). In some embodiments, cell seeding density for each cell type (10⁶-10⁸ cells/ml) ranges based on physiological data at different locations in the LNs. In some embodiments, after forming the LN paracortex, B cell follicles compartments, and 3D blood vessels after 5 days, DCs are loaded from the microfluidic lymphatic vessel channels (one or more reservoirs 136) and enter the paracortex region (inner region 116) with perfusion of fluids with a digitally controllable pressure pump, to induce adaptive immune response on device 100. The flow rate of lymph fluid through human LNs is highly variable, ranging from a few to tens of microliters per hour. Actual rates vary based on factors like health, node location, and physiological conditions [Zawieja D C., Lymphat Res Biol. 2009; 7(2):87-96].

In some embodiments, cells from human LN are used within device 100 as follows: Mesenteric LNs dissected from non-neoplastic gastrointestinal resection specimens (e.g., inflammatory bowel disease and diverticular disease with dysplasia) are utilized to construct the disclosed device 100. In some embodiments, the tissue dissociation process involves digestion into a single-cell suspension using 0.8 mg/ml Dispase (Stemcell) and 0.2 mg/ml Collagenase P (Sigma-Aldrich). Then cell classification is conducted using FACSAria IIu SORP (BD Biosciences). In some embodiments, the collected cell populations encompass T cells (CD45+, CD3+), B cells (CD45+, CD19+), DCs (CD45+, MHC+, and CD11c+), FRCs (CD45−, CD31−, PDPN+) and FDCs (CD21+, CD35+). In some embodiments, T cells and FRCs are loaded into the paracortex compartment (e.g., inner region 116) and load B cells and FDCs into the B cell follicle regions (one or more outer regions 118) that are partitioned by spaced PDMS micropillars (e.g., plurality of micropillars 114).

### Methods

Device 100 and methods disclosed herein can be used to model response and/or effectiveness of therapy, or in other examples, to model certain disease states related to the LNs, such as lymphatic diseases. In some embodiments, device 100 can be adapted to replicate the microenvironment from patient-specific cells such that treatment conditions can be modeled and tailored to individual patients. In some embodiments, the devices are suitable for evaluating any therapy including, but not limited to, vaccine, mRNA vaccine, inactivated vaccine, adenovirus-based vaccine, small molecule, protein, nucleic acid molecule, anti-cancer drug, immunotherapy, chemotherapy, radiation therapy, chemoradiation therapy, and targeted therapy on a patient-specific basis.

Aspects of the present invention relate to a method of measuring or evaluating an immune response (e.g., an adaptive immune response). Referring now to FIG. 1E, shown is an exemplary method 200 of measuring an immune response comprising the steps of: 202 providing an LN on a chip device (e.g., device 100), 204 administering at least one treatment to the device, and 206 determining immune responsiveness based on at least one measured change on the device.

Aspects of the present invention relate to a method of measuring or evaluating a response to a treatment or therapy. In some embodiments, the method of measuring or evaluating a response to a treatment or therapy comprises the steps of providing an LN on a chip device (e.g., device 100), administering at least one treatment or therapy to the device, and determining treatment responsiveness based on at least one measured change on the device. Exemplary procedures and further detail for conducting or performing the assays, measurements, and determining treatment responsiveness can be found in the examples section below.

In some embodiments, any disclosed method further comprises the step of administering at least one adjuvant in combination with the at least one treatment. In some embodiments, at least one adjuvant is selected from the group consisting of: AS03, CpG Oligodeoxynucleotides, squalene, monophosphoryl Lipid A, aluminum salts and MF59, or any combinations thereof.

In some embodiments, the step of administering at least one treatment and/or adjuvant to the device comprises administering the at least one treatment and/or adjuvant to central chamber 108 and/or one or more reservoirs 136. It should be appreciated that the at least one treatment and/or adjuvant may comprise a fluid, solid, liquid, gas, or mixture, or any combination thereof, of treatments, therapies and/or adjuvants disclosed herein. In some embodiments at least one treatment may be administered through one or more openings 110 of device 100 into one or more specific areas or regions of the device (e.g., reservoirs 136, inner region 116 (paracortex region), outer region 118 (follicle region), circumferential region 120 (subcapsular sinus region). The at least one treatment may be administered using any device or method known by one of normal skill in the art, including, but not limited to, a syringe, a needle, a dropper, a pipette, applicator bottle, a tube, conduit, catheter, or the like, or any combination thereof.

In some embodiments, the at least one treatment is selected from the group consisting of: vaccine, mRNA vaccine, inactivated vaccine, adenovirus-based vaccine, small molecule, protein, nucleic acid molecule anti-cancer drug, immunotherapy, chemotherapy, radiation therapy, chemoradiation therapy, and targeted therapy on a patient-specific basis.

In some embodiments, the measured change comprises any of: antibody secretions, cell migration, cell proliferation, cell activation, cell infiltration, cell speed, cell trajectory, cell distance, cell position, cell motility, cell maturation, cell maturation efficiency, cell maturation efficiency, cell phenotype, cell differentiation, cell concentration, cell recruitment, cell migration within one region (e.g., the inner region), cell migration from one region to another (e.g., the inner region to the interfollicular region), pH in the central chamber, pH in a culture medium, number, size and spatial distribution of germinal centers, number, size and spatial distribution of germinal centers in the one or more outer regions.

In some embodiments, the at least one measured change comprises: antibody secretion (e.g., immunoglobulin such as IgG, IgM, IgE, IgD, or secretion levels of total IgG, secretion levels of total IgM), and T cell migration from the paracortex region to the interfollicular region. In some embodiments, the at least one measured change further comprises cytokine concentration.

In some embodiments, Fluzone® Quadrivalent Influenza Vaccine (2017-2018) is added to central chamber 108 of device 100 at a dilution ratio of 1:10,000. In some embodiments, the secretion of vaccine-specific antibodies (H1N1, H3N2, Influenza B) is measured in or from device 100 (e.g., from the effluent of device 100) over 14 days using ELISA. In some embodiments, a higher vaccine-specific antibody secretion at day 14 is expected or anticipated in the vaccine group, indicating a vaccine response. In some embodiments, simultaneously, images of GL7+ germinal center-like cells are captured or imaged within device 100 using a confocal microscope (Nikon), and statistical analysis is formed using ImageJ. In some embodiments, a significant increase in the number of GL7+ germinal center-like cells in the vaccine group is expected or anticipated, indicating a vaccine response.

Aspects of the present invention relate to simulating one or more disease states on the disclosed LN on a chip device. In some embodiments, device 100 is configured to simulate one or more disease states. In another example, any disclosed method may further comprise the step of simulating one or more disease states on the device. In some embodiments, the one or more disease states comprise lymphatic diseases, autoimmune disorders, cancer, acute lymphadenitis, lymph node tuberculosis, rheumatoid arthritis, systemic lupus erythematosus, Hodgkin lymphoma, Non-Hodgkin lymphoma, and metastatic cancer.

Aspects of the present invention relate to culturing one or more patient or subject cells in or on the disclosed LN on a chip device. In some embodiments, any disclosed method further comprises the step of culturing one or more subject or patient derived cells on device 100. In some embodiments, the step of culturing one or more cells on device 100 comprises culturing at least a portion of device 100 with one or more cells from a subject, patient-specific organoids and/or patent derived organoids (PDOs). For example, in some embodiments, one or more cells from a subject are seeded and cultured within central chamber 108 of device 100. In some embodiments, the one or more cells from a subject are seeded simultaneously with the other cells on device 100 (e.g., paracortex cells 124, follicle cells 126, interfollicular cells 130, sinus cells 132).

In some embodiments, any disclosed method comprises the step of providing a fluid flow to device 100 to simulate lymph flow within the device. In some embodiments, the step of providing a fluid flow comprises providing a fluid flow to at least a portion of device 100 ranging between from 0-120 μL/h to simulate lymph flow within the device. For example, in some embodiments, the step comprises providing a fluid flow of about 60 μL/h to one or more reservoirs 136 and/or central chamber 108. In some embodiments, a chip culture medium is provided or injected to device 100 with one or more fluid flows. In some embodiments, the chip culture medium comprises any of EGM™-2 (Lonza), RPMI-1640 (Gibco), and/or HLF (Sciencells), or any combinations thereof.

In some embodiments, the disclosed methods allows one to determine cellular and transcriptomic signatures by gene expression (scRNAseq). For example, in some embodiments, any disclosed method comprises the step of sampling or isolating the cells in device 100 from one or more areas or regions in the device (e.g., inner region 116 (paracortex region), outer region 118 (follicle region), circumferential region 120 (subcapsular sinus region), one or more channels 122 (interfollicular region) for evaluating one or more genomic, transcriptomic and/or proteomic signatures. In some embodiments, variable cellular states and subsets of FRCs and immune cells can be stratified using the single-cell transcriptome sequencing data, enabling a detailed understanding of cellular heterogeneity. In some embodiments, proteomic (cytokine and matrix metalloproteinase) measures can also be examined. In some embodiments, the data generated can be examined in context of histopathological characteristics from the original biopsy specimens.

In some aspects, the present invention relates to a method of evaluating vaccine effectiveness using an LN on a chip device (e.g., device 100) wherein the method comprises five key functional indices normalized with range of 0-1 to comprehensively evaluate vaccine effectiveness. The five functional indices are as follows: 1. Immune cell phenotypes: DC maturation (CD80, CD83, and HLA-DR), T cell proliferation (ki-67) and differentiation types (CD4+ Th cells, CD8+ cytotoxic T cells), B cell differentiation types (CD27+ memory B cells, CD138+ plasma cells); 2. Lymphocyte trafficking: DC recruitment and T cell migration; 3. Proteomic characterization related to immune cell homing (CCL19, CCL21), T cell activation (IFN-γ, IL-10, IL-8, IL-2), T cell migration (e.g. CCL17), and B cell differentiation (IL-4, IL-6); 4. Number and size of germinal centers (GL7+); 5. Antibody secretions: total IgG, IgM, and vaccine-specific neutralizing antibody concentrations.

Aspects of the present invention relate to a method of testing the effects of different vaccines (e.g. human seasonal influenza virus vaccine, rabies virus vaccine) and adjuvants (e.g., MF59, AS03) in LNs, wherein at least one measured change comprises any of: 1. Cell-cell communications: DCs and T cells, Tfh and naïve B cells. Immunofluorescence can be used to visualize T cell types (CD4+, CD8+), B cell types (CD27+ memory B cell, CD138+ plasma cell) and germinal centers (GL7+) in vitro. 2. Cytokines: ELISA to track cytokine changes related to T cell activation (IFN-γ, IL-10, IL-2) and B cell differentiation (IL-4, IL-6) across populations. 3. Lymph fluid impact: The chip allows one to compare in vitro LN niche interactions (cell-cell communication, cytokine changes) under fluid vs. static conditions. 4. Antibody secretion analysis: The chip allows one to use ELISA to measure total, IgG, IgM, and vaccine-specific antibodies, assessing vaccine effects across different population groups (age, gender, race etc.). Further descriptions of the disclosed device and methods are discussed in the examples below.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Tissue-Engineered Organotypic Human Lymph Node for Adaptive Immunity Modeling and Vaccination Effectiveness Assessment

Vaccine development is an exceedingly intricate and time-consuming process [B. C. Buckland, Nature Medicine 11, S16-S19 (2005)]. The lack of comprehensive preclinical data and a dearth of precise information on the correlates of immune protection have frequently led to vaccine products failing in clinical trials [B. Pulendran, M. M. Davis, Science (New York, N.Y.) 369, (2020)]. To address this issue, it is crucial to develop more relevant animal models and to collect and analyze human samples extensively. However, interspecies differences between animal models and humans hinder accurate replication of immune responses following vaccination [A. C. Walls et al., Cell Rep 40, 111299 (2022)]. Furthermore, ethical and safety concerns surrounding paid recruitment of clinical volunteers have faced widespread criticism [D. Calina et al., Journal of Faculty of Pharmacy, Tehran University of Medical Sciences 28, 807-812 (2020)]. These underscore the pressing importance of devising new vaccine testing models. Lymph nodes (LNs), as secondary immune organs, play a pivotal role in capturing vaccine antigens and generating both memory cells for long-term immunity and plasma cells for antibody secretion [E. Moysi et al., Expert Rev Vaccines 21, 633-644 (2022)]. The adaptive immune processes that occur in LNs after vaccination form the foundation for the body's enduring immunity [I. J. Amanna, M. K. Slifka, Curr Top Microbiol Immunol 428, 1-30 (2020).]. Nevertheless, there remains a critical unmet need for humanized models that simulate adaptive responses within human lymph nodes (LNs) and investigate the variations in vaccine responses among different populations. The disclosed device and method is the result of significant experience in the field of bioengineered microfluidic vascularized immunocompetent organ-on-a-chip devices, which facilitate the study of cellular interactions within tissue microenvironments in vitro [M. T. Witkowski et al., Cancer Cell 37, 867-882 e812 (2020); Z. Zhang et al., Small Methods 5, (2021); B. Ma et al., Biosensors & Bioelectronics 230, 115247 (2023); X. Cui et al., Elife 9, (2020); Ma et al., Science Advances 6, (2020)]. Disclosed herein is a human “Lymph node-on-a-Chip” microphysiological system (i.e., LN on a chip device) with microanatomic organization of T lymphocytes, B lymphocytes, blood-derived dendritic cells (DCs), human LN fibroblastic reticular cells (FRCs), follicular dendritic cells (FDCs), and vascular cells that replicates the in vivo physiology of the human LN microenvironment. The incorporation of bioengineered vasculature within the system allows for continuous perfusion, offering a unique opportunity to examine cell behaviors and signals originating from the peripheral bloodstream, as well as those within the stromal environment. The disclosed device is invaluable for investigating immune cell recruitment into the tissue and for testing the effects of cytokines.

Engineering a 3D ‘LN-on-a-Chip’ in vitro organotypic platform that mimics in vivo LN niche: the disclosed microphysiological system (i.e., LN on a chip device, “LN chip” or “chip”) recapitulates the human stromal cellular organization and immune functional cellular organization in the LN niche. The disclosed device is engineered with real-time imaging capabilities and integrates continuous perfusion fluid control that enables precise control over biophysical and lymphatic flow within the LN microenvironment formed on the device. Through the strategic placement of micropillars, the chip was divided into functional units mirroring key structural components of a natural LN, including the paracortex (T cell area), the B cell follicle (B cell area), the subcapsular sinus (emulating the lymph flow area), and the blood vessel (emulating sites of immune cell recruitment). The LN stromal reticulum was reconstructed, and the key LN stromal spatial organization was analyzed on-chip, showing T cell region FRCs (TRCs, gp38⁺,CD157⁺, and CD31⁻) to be at highest density in the paracortex area, perivascular reticular cells (PRCs, gp38⁻,CD157⁺, and CD31⁻) surround high endothelial venules (HEVs, blood vessel area), and follicular dendritic cells (FDCs, CD35⁺, and CD31⁻) to be located at maximum density in the B cell follicle. The disclosed device (and method of use thereof) allows one to dissect the heterogeneity of stromal microenvironments between different populations (age, health status, etc.). Further, the device and method allows one to determine cellular and transcriptomic signatures by gene expression (scRNAseq). Variable cellular states and subsets of FRCs and immune cells can be stratified using the single-cell transcriptome sequencing data, enabling a detailed understanding of cellular heterogeneity. Proteomic (cytokine and matrix metalloproteinase) measures can also be examined. Data generated can be examined in context of histopathological characteristics from the original biopsy specimens.

Modeling adaptive immune processes within LNs to assess vaccine efficacy and vaccination strategies: the LN on a Chip device for adaptive immune modeling can be used with immune cells derived from different populations, including healthy people of different ages, different health states (e.g., hypertension, diabetes, etc.). Using the disclosed device and method, it can be tested on chip whether aging and chronic disease-induced reductions in vaccination efficiency are associated with changes in the LNs niche [M. Cakala-Jakimowicz et al., Cells 10, (2021); J. Chen et al., Trends Mol Med 28, 1100-1111 (2022)], and to evaluate the effectiveness of vaccines and vaccination strategies in different populations. Using the disclosed device, the seasonal influenza virus inactivated vaccine (ATCC BEI) and Covid-19 inactivated virus (ATCC BEI) can be used to characterize the key processes of the adaptive immune response of the LN, including DCs-mediated antigen presentation, T cell activation, germinal center formation (B cell differentiation), and secretion of antigen-specific antibodies. In some embodiments, the evaluation of the effects after vaccination can be determined by the following factors: DC-mediated antigen presentation efficiency, T cell activation, B cell differentiation (CD27⁺ memory cells, CD138⁺ plasma), number and size of germinal centers (GL7⁺), changing levels of cytokines (IFN-γ, IL-10, IL-2, IL-4, IL-6) and neutralizing antibody concentrations. The effectiveness of vaccine adjuvants such as aluminum salts and MH59 on vaccination can also analyzed. The disclosed device and method provides a means for the discovery of better vaccination strategies for susceptible populations by combining vaccine adjuvants. The disclosed device and method provides a robust and easily reproducible LN-on-a-Chip model that can be used to study adaptive immune mechanisms and evaluate vaccine efficacy and vaccination strategies in large populations.

Vaccines play an essential role in controlling and preventing infectious diseases in humans [E. Dube et al., Annu Rev Public Health 42, 175-191 (2021); M. Jeyanathan et al., Nat Rev Immunol 20, 615-632 (2020); D. Y. Cassandra Pingali et al., Morbidity and Mortality Weekly Report 70, 1183-1190 (2021)]. Successful vaccination requires efficient delivery of antigenic substances to induce a strong therapeutic or protective immune response [S. Sell, Expert Rev Vaccines 18, 993-1015 (2019); L. Sompayrac, (2019)]. Because vaccine antigens primarily activate the adaptive immune system, which remembers and eliminates the antigen as a foreign invader, the efficacy of the vaccine depends on the extent to which the vaccine enhances the adaptive immune response in the body [Y. Ding et al., Adv Drug Deliv Rev 179, 113914 (2021); J. Leleux et al., J Control Release 219, 610-621 (2015)]. Adaptive immune response is mainly initiated in LN, which serve as vital hubs for adaptive immune-related lymphocytes, including antigen-presenting cells (APCs), CD4⁺ helper T cells, CD8⁺ T cells, and B cells [S. M. Grant et al., J Cell Sci 133, (2020)]. However, depletion of CD4⁺ T cells, reduced germinal centers, and LN extracellular matrix (ECM) fibrosis due to aging and chronic disease can lead to delayed immune responses and hinder the generation of memory B cells, affecting vaccine efficacy across populations [M. Cakala-Jakimowicz et al., Cells 10, (2021); J. Chen et al., Trends Mol Med 28, 1100-1111 (2022); O. Ahmadi et al., ANZ J Surg 83, 612-618 (2013)]. Therefore, understanding the impact of the LN niche on adaptive immunity is critical for developing effective vaccines and new vaccination strategies tailored to different populations.

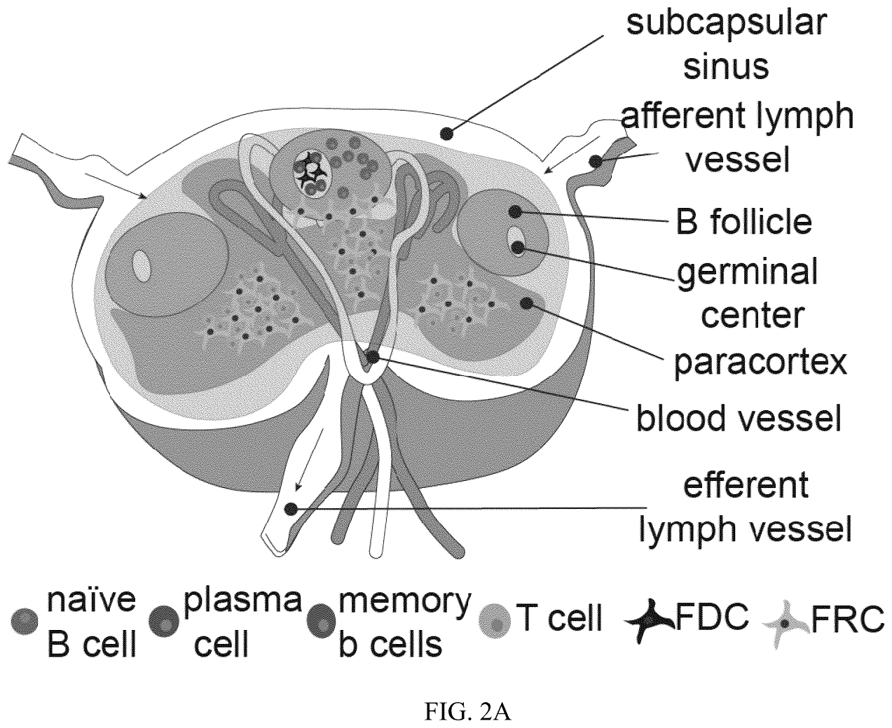

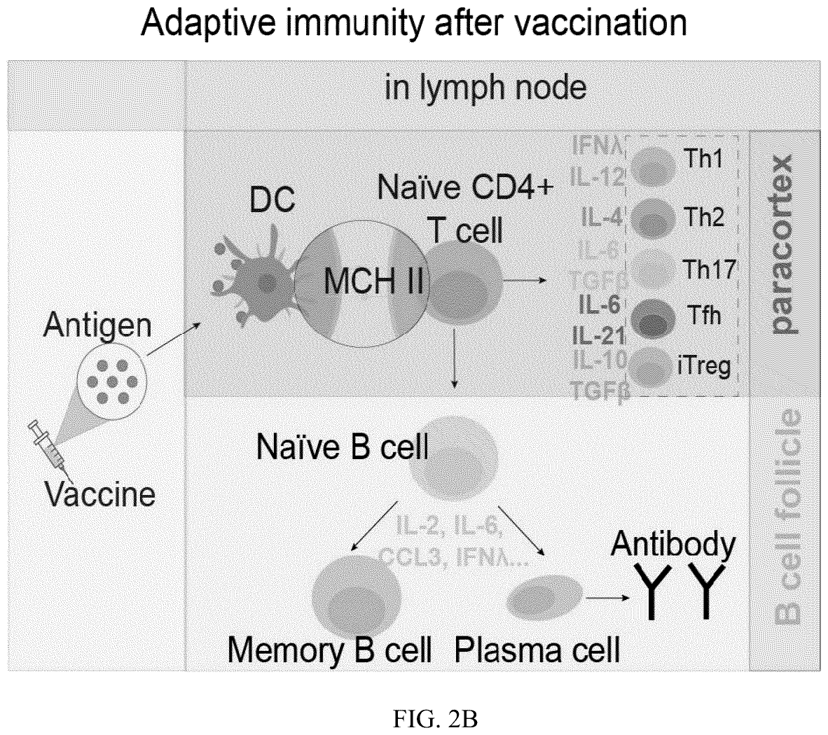

The LN is a highly structured organ, with its essential functions reliant on the unique spatial sorting of lymphocytes and stromal cells, as well as the chemokines that drive the signaling cascades underlying the immune response (see FIG. 2A and FIG. 2B) [B. E. Burrell et al., Front Immunol 2, 64 (2011); S. Liao, P. Y. von der Weid, Semin Cell Dev Biol 38, 83-89 (2015); J. E. Chang, S. J. Turley, Trends Immunol 36, 30-39 (2015); Y. Chen et al., Ace Chem Res 53, 2055-2067 (2020)]. FIG. 2A is a schematic diagram depicting the structure and function of human lymph nodes. FIG. 2B is a schematic diagram of the adaptive immune process in LN after vaccination. Developing models of LNs that accurately replicate their intact immune niche, while describing the dynamics and organization of this organ, is a significant challenge in human adaptive immunity research. To date, no in vitro model system or device of LNs has been able to simulate the complete dynamic process of adaptive immunity [T. Ozulumba et al., Front Immunol 14, 1183286 (2023)]. While humanized animal models are currently available for vaccine evaluation, they come with certain limitations. These include inefficient cooperation between T cells and B cells, reduced efficiency in immunoglobulin class switching, and a limited number of LNs [T. T. Murooka, T. R. Mempel, J Infect Dis 208 Suppl 2, S137-144 (2013); C. Zhang et al., Pathogens 12, (2023); R. S. Herati, E. J. Wherry, Cold Spring Harb Perspect Biol 10, (2018)]. Additionally, 3D organoid cultures partially replicate organ complexity but struggle to model lymphatic fluid dynamics and vascular structure, which are essential for immune cell entry into LN and adaptive responses [A. Purwada et al., Biomaterials 63, 24-34 (2015)]. The lack of anatomical and microphysiological control in current models leads to less controllable and reproducible experiments [A. Purwada et al., Biomaterials 63, 24-34 (2015)]. Furthermore, existing organotypic chips for LNs tend to focus on specific regional LN structural functions, such as DCs and T cell migration influenced by cytokines and immune cells homing influenced by lymph fluid [K. G. Birmingham et al., iScience 23, 101751 (2020); N. Hallfors et al., Bioengineering (Basel) 8, (2021); P. Moura Rosa et al., Lab Chip 16, 3728-3740 (2016)]. These models lack full functional compartmentalization and key cellular LN components, hindering complete adaptive immunity replication. The disclosed biomimetic immunocompetent LN-on-a-chip in vitro model (i.e., LN on a chip device) replicates the intricate structure and immune niche of natural LNs, effectively overcoming the limitations of current model systems in vaccine evaluation.

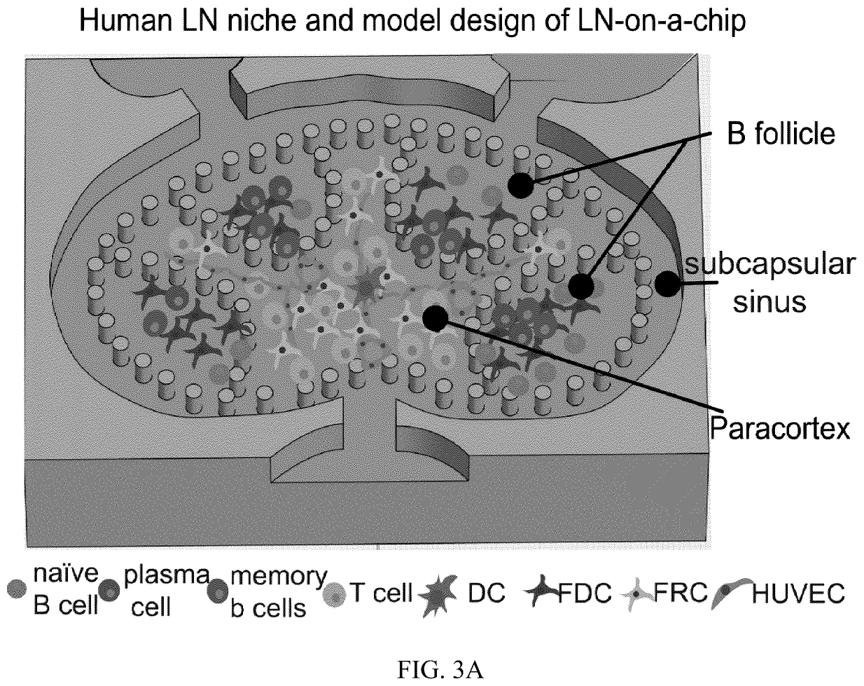

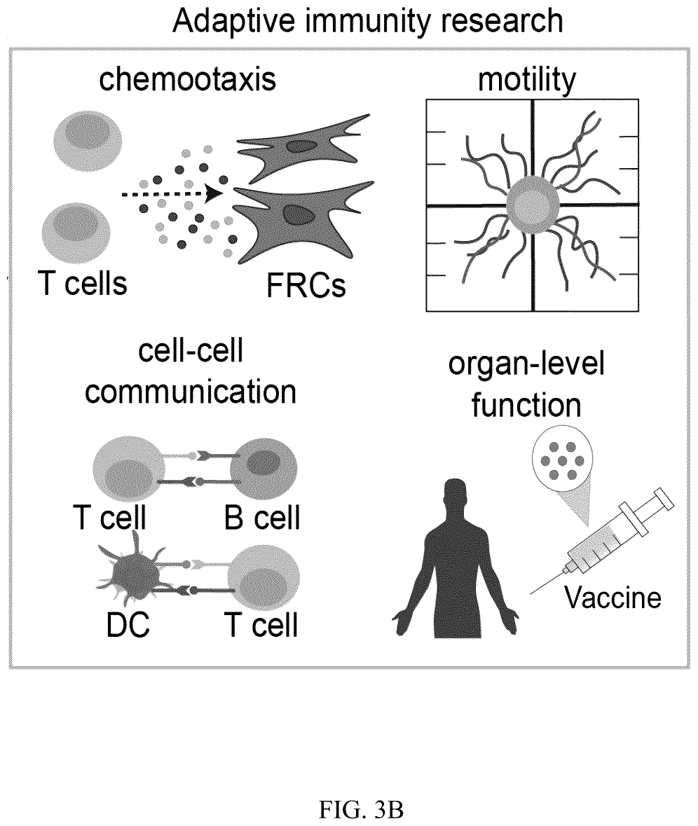

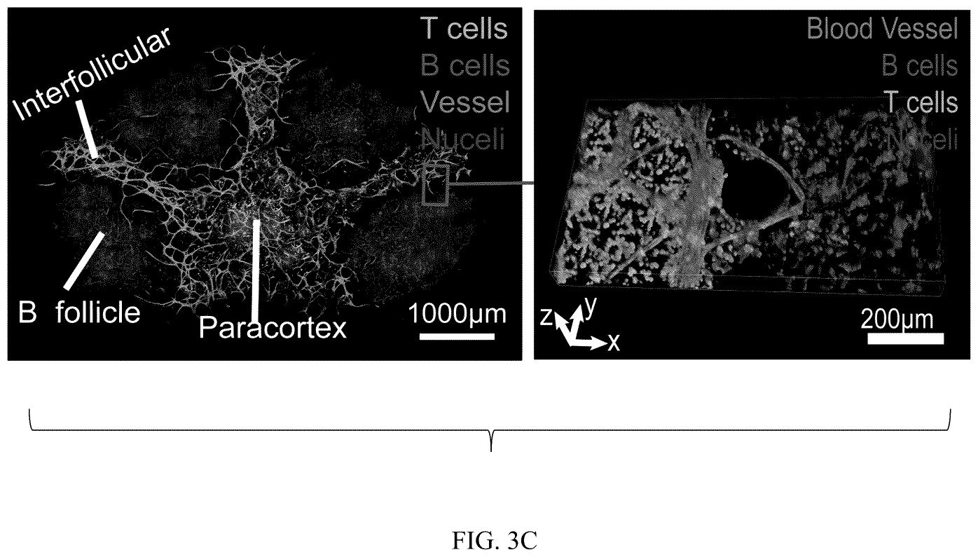

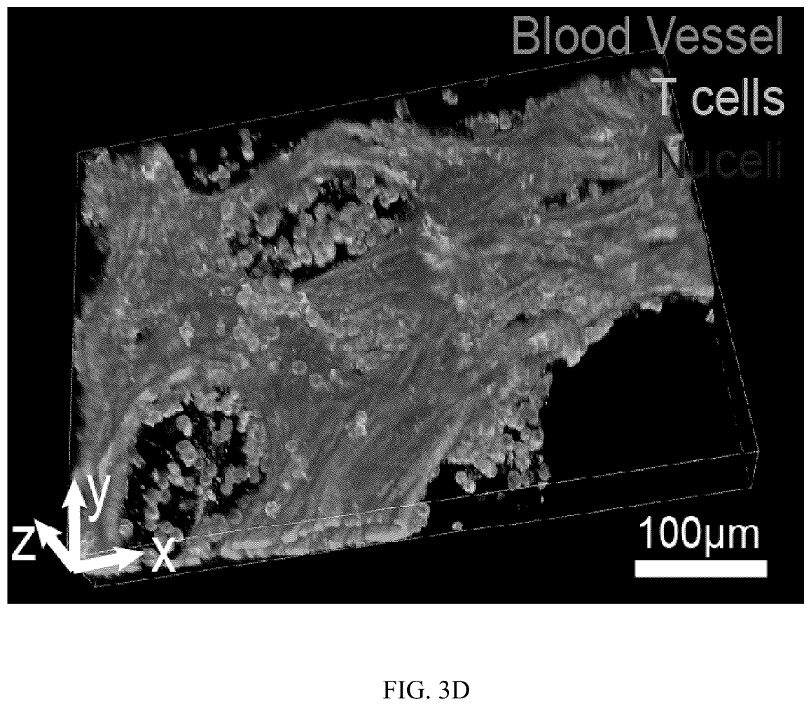

An engineered 3D platform that mimics the structure of the native LN niche (see FIG. 3A through FIG. 3D) was developed. FIG. 3A depicts an exemplary LN on a chip device with a human LN in vitro model comprising an organotypic microfluidic chip design. FIG. 3B depicts various adaptive immunity research techniques (e.g., adaptive immune response research) enabled on the disclosed LN-on-the-chip device. FIG. 3C shows images of a whole scan of the LN-on-a-chip device. FIG. 3D is an image showing blood vessels in the paracortex region of the disclosed LN-on-a-chip device. The LN-on-a-Chip device with LN niche model was constructed with major LN immune functional compartments (paracortex, follicle, subcapsular sinus) with biomimetic ECM, key immune cell populations (T cells, B cells, dendritic cells) and stromal cells (fibroblast and lymphatic vessels), as well as continuous perfusion of lymph fluids in the LN niche. In the disclosed work, an organotypic and immunocompetent human “LN-on-a-Chip” microphysiological system (i.e., LN on a chip device) was developed that replicates the complex structure and immune function of native LN. Modeling and decoding the LN microenvironment using the disclosed device provides a new approach to modelling human adaptive immune response and offers a powerful tool for vaccine evaluation, screening, and new vaccination strategy development.

Novel biomimetic “LN-on-a-Chip” organotypic modeling system: the disclosed device provides a platform for investigating human adaptive immune mechanisms and assessing vaccine efficacy and vaccination strategies. Through the incorporation of human cells in finely crafted microenvironments that closely mimic physiological conditions, microfluidic organ-on-a-chip technologies facilitate the recreation of essential functions and structures found in miniature human tissues [C. Ma et al., Trends Pharmacol Sci 42, 119-133 (2021)]. However, there is no organ-on-a-chip model of the human LN capable of simulating a complete adaptive immune response in vitro. The disclosed LN-on-a-Chip in vitro model recapitulates human LN functional structural compartmentalization, vasculature, stromal and immune niches, with biological and biophysical controls. In some embodiments, cell-cell communication during adaptive immunity can be analyzed with the disclosed device. Further, immune cells from different populations may be used to construct LNs on the chip for evaluating vaccine efficacy and vaccination strategies tailored to diverse populations. The disclosed organ-on-chip model (i.e., LN on a chip device) simulates the complete adaptive immune process (including DCs transmitting antigen signals to CD4+ T cells, and Tfh assisting naïve B cells differentiation into memory B cells and antibody-secreting plasma cells) of a typical LN (e.g., a human LN).

In the disclosed example, a controllable, reproducible, and easily accessible human biomimetic LN model was developed for the evaluation of vaccine efficacy and vaccination strategies. In some embodiments, the disclosed device (see FIG. 3A through FIG. 3D) allows control of various biological parameters (e.g., cell types, tissue structures, ECM composition, sustainably lymphatic system perfusion), and real-time visualization of adaptive immunity (e.g., DCs migration, T cell activation, B cell differentiation). The device is easy to set up and is compatible with high-throughput microarray analysis (e.g., molecular, cellular, and histological characterization). Additionally, the device allows for subsequent cell retrieval for in-depth genetic analysis, such as scRNA-seq. The disclosed device supports longitudinal (2-3 weeks of real-time tracking and imaging) and systemic (molecular, cellular, and tissue-scale) analysis to understand the mechanisms and functions of the LN niche during adaptive immunity. In some embodiments, the disclosed LN-on-a-Chip device is used to construct the LN niche of different populations to further explore the impact of the LN niche on adaptive immunity. In some embodiments, the disclosed device and method provides a powerful tool for investigating the adaptive immune mechanisms of human LN and for advancing vaccine development and vaccination strategies.

Engineered 3D ‘LN-on-a-Chip’ in vitro organotypic platform that mimics in vivo LN niche: increasing evidence reveals the important role of the LN tissue microenvironment in the adaptive immune process [S. Jalkanen, M. Salmi, Nat Rev Immunol 20, 566-578 (2020); J. Lian, A. D. Luster, Curr Opin Cell Biol 36, 1-6 (2015)]. A better understanding of LN functional structural compartmentalization, vasculature, immune cell communication (e.g., DCs and T cells, T helper cells and B cells), and extracellular signals (e.g., ECM, lymph fluid dynamics) enhances the understanding of human adaptive immune mechanisms. To address this, an LN-on-a-Chip microphysiological system (i.e., LN on a chip device) was derived from human immune cells to accurately reconstruct the highly organized immune processes of the LN niche in vitro.

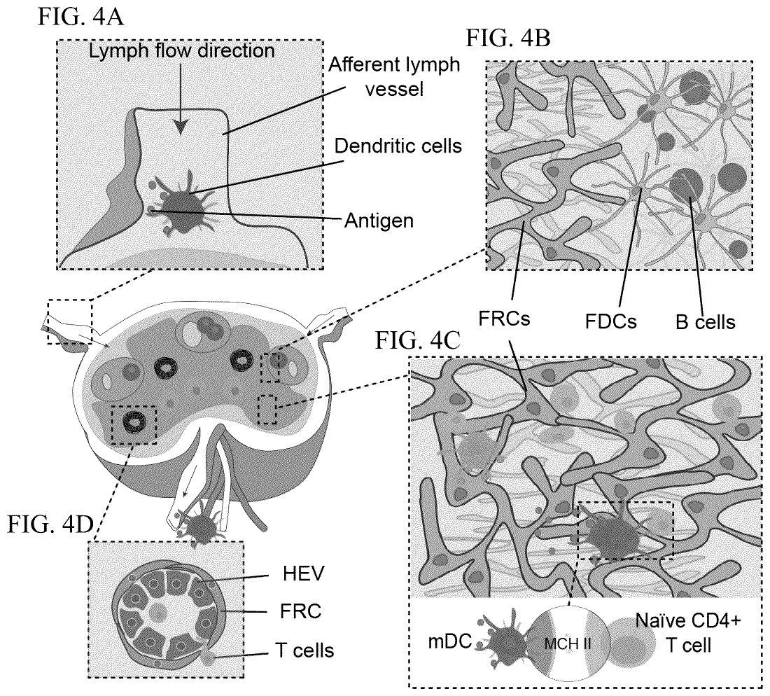

Chip design: The microfluidic LN-on-a-chip model was constructed with major functional immune niche compartments of LNs (see FIG. 3A through FIG. 3D), including the B cell follicles, paracortex (T cell zone), supported with key stromal niche cells (FRC, FDCs, blood vessels) and biomimetic ECM. Specifically, the paracortex is the T cell region where DCs present antigens to T cells and activate adaptive immune responses (see FIG. 4A through FIG. 4F) [B. C. Duckworth et al., Immunol Rev 306, 76-92 (2022)]. FIG. 4A through FIG. 4D is a diagram depicting an LN organization. FIG. 4A shows that DCs arrive at the LN via afferent vessels and then migrate into the cortex. FIG. 4B shows that B cells are located in follicles and interact with FDCs. FIG. 4C shows that T cells are in the paracortex to interact with DCs, supported by FRCs. FIG. 4D shows that DCs migrate on reticular fibers to the HEVs, where they interact with naïve lymphocytes entering the LN from the HEV, and activated B and T cells crawl along the medullary sinus to leave the LN. The B cell follicles are areas of B cells within LN that are involved in the production of antibodies and immune responses to specific antigens [C. Young, R. Brink, Immunity 54, 1652-1664 (2021)]. In some embodiments, the disclosed device will support 2-3 weeks of continuous monitoring and real-time imaging, enabling comprehensive, systematic testing of adaptive immune response within a physiologically relevant LN microenvironment. In some embodiments, in addition to these cellular compartments, the device also integrates with surrounding microfluidic channels mimicking lymphatic vessels and subcapsular structures that allow continuous perfusion of fluids into the LN niche and study of DCs recruitment during adaptive immune response [S. A. Elmore, Toxicol Pathol 34, 425-454 (2006)].

Chip microfabrication: The PDMS-based microfluidic chip was fabricated using a standard soft lithography replica-molding method [C. Ma et al., Science Advances 6, (2020)]. The different culture compartments of the chip are partitioned by regularly spaced PDMS micropillars that confine cell-embedded hydrogels. T cells, and FRCs were loaded into the paracortex area (T cell zone), and B cells and FDCs were loaded into the B cell follicle region in the LN chip. In addition, 3D blood vessels mimicking high endothelial venule (HEV) located in the T cell zone of the LN chip were created for lymphocytes trafficking, by compartmentalizing human primary endothelial cells (HUVECs) in the paracortex channels of the immune compartments (see FIG. 3C). Cell seeding density was optimized for each cell type (10⁶-10⁸ cells/ml) based on physiological data at different locations in the LNs. After forming the LN paracortex, B cell follicles compartments, and 3D blood vessels after 5 days (see FIG. 3D), DCs were loaded from the microfluidic lymphatic vessel channels, and entered the paracortex region with perfusion of fluids with a digitally controllable pressure pump, to induce adaptive immune response on chip. In some embodiments, the flow rate of lymph fluid through human LNs is highly variable, ranging from a few to tens of microliters per hour. In some embodiments, actual rates vary based on factors like health, node location, and physiological conditions [D. C. Zawieja, Lymphat Res Biol 7, 87-96 (2009)]. Flow rate of the perfusion system from 0-120 μL/h was optimized to simulate lymph flow within the lymphatic system.

Cell samples: PBMC-derived immune cells were used to build the disclosed LN chip. Specifically, naïve B cells, T cells, and monocytes were isolated from human PBMCs using magnetic beads Cell Isolation Kits (StemCell Technologies). DCs were differentiated from isolated CD14⁺ monocytes via treating with 400 ng/mL GM-CSF and 250 ng/mL IL-4 in complete RPMI medium for 5-6 days. Additionally, human FRCs (Sciencell) and HUVEC (Lonza) were used as stromal cells and for vascularization of the chip. FDCs were obtained, and the FDC cell line, KY6, were isolated from discarded human tonsils and immortalized by retroviral transduction with pBABE_TERT.Hygro, P53DD_Thy1.1 and CDK4_R24C_Thy1.1 as described in [R. Caeser et al., Nat Commun 10, 4543 (2019)].

Alternatively, we can use primary cells from human LN into our model as follows: Mesenteric lymph nodes dissected from non-neoplastic gastrointestinal resection specimens (e.g., inflammatory bowel disease and diverticular disease with dysplasia) will be utilized to build our LN chip model. Specifically, the tissue dissociation process will involve digestion into a single-cell suspension using 0.8 mg/ml Dispase (Stemcell) and 0.2 mg/ml Collagenase P (Sigma-Aldrich). Then cell classification will be conducted using FACSAria IIu SORP (BD Biosciences). The collected cell populations will encompass T cells (CD45⁺, CD3⁺), B cells (CD45⁺, CD19⁺), DCs (CD45⁺, MHC⁺, and CD11c⁺), FRCs (CD45⁻, CD31⁻, PDPN⁺) and FDCs (CD21⁺, CD35⁺). We will load T cells and FRCs into the paracortex compartment and load B cells and FDCs into the B cell follicle regions that are partitioned by spaced PDMS micropillars.

ECM components: The ECM of natural LNs is mainly composed of type I, III and IV collagen, laminin, albumin and fibronectin, etc. [F. N. Morgado et al., Cells 9, (2020)]. In order to simulate the ECM of natural LNs, a mixture of Matrigel (rich in collagen and laminin) and fibrin hydrogel (Sigma) were used as the ECM of this model. Fibrin hydrogel is a cytoplasmic matrix that is widely used in the development of vascularized organs in vitro [B. T. Wonski et al., Bioengineering (Basel) 10, (2023)]. Culture optimizations: Since multiple types of cells were cultured in the system, culture media was optimized with a mixture of media for HUVEC (EGM™-2, Lonza), FRC (2301, Sciencells) and lymphocytes (RPMI1640, GIBCO) at different ratios (e.g., 1:1:1, 2:1; 1, or 1:2:1). In some embodiments, chemokines are added to promote vascularization (e.g. VEGF) and germinal center formation (e.g. IL-4, and anti-CD40Ab). In some embodiments, 50% of the culture medium is replaced with fresh medium every 2 days. In some embodiments, perfusion is tested to see if static culture fails to maintain primary cells.

Characterization of the spatial organization of LN niche on chip: the chip was immunostained with CD3, CD19, and CD31 respectively on day 10 to prove successful forming the LN paracortex (T cell zone), B cell follicles compartment, and 3D blood vessels (HEVs). The localization and density of T cells (CD4⁺ Helper T cells, CD8⁺ cytotoxic T cells) and PDPN⁺ FRCs in the paracortex (T cell zone), CD19⁺ B cells and CD35⁺ CD21⁺ FDCs in the B cell follicle region of the LN chip was determined by immunostaining (see FIG. 4A through FIG. 4D).

Study of the LN stromal niche on Chip: LN stromal cells, including the HEVs, specialized FRCs and FDCs, form reticular microenvironments to support adaptive immune responses not only by structural components, but also by secretion of soluble factors that guide, retain, and promote immune cells in specific zones of the LN [L. B. Rodda et al., Immunity 48, 1014-1028.e1016 (2018)]. Depending on their localization in the LN, these cells display heterogeneous properties supporting the different activities of the adaptive immune response.

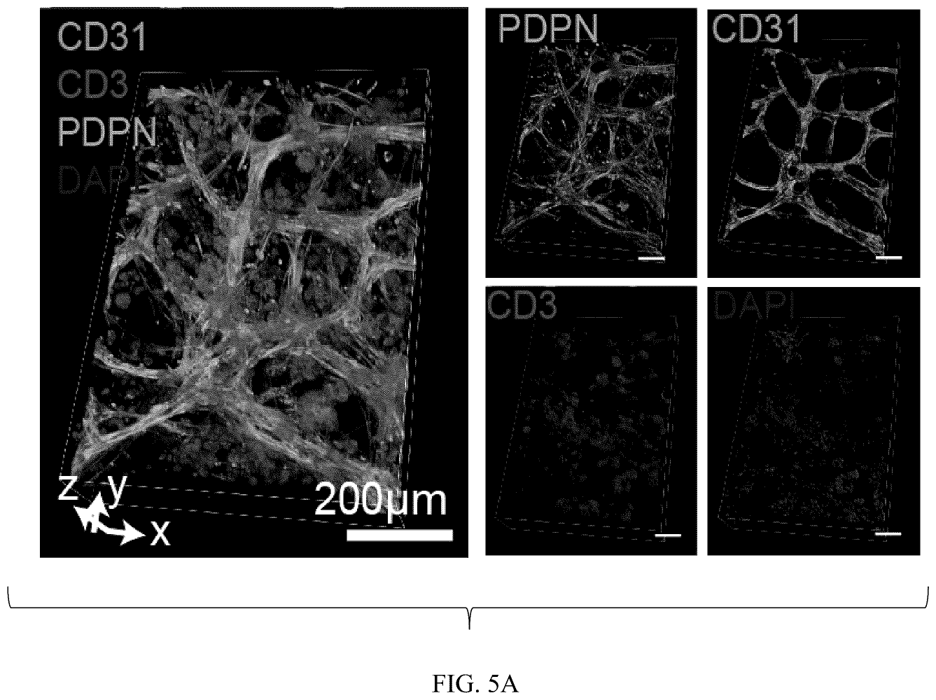

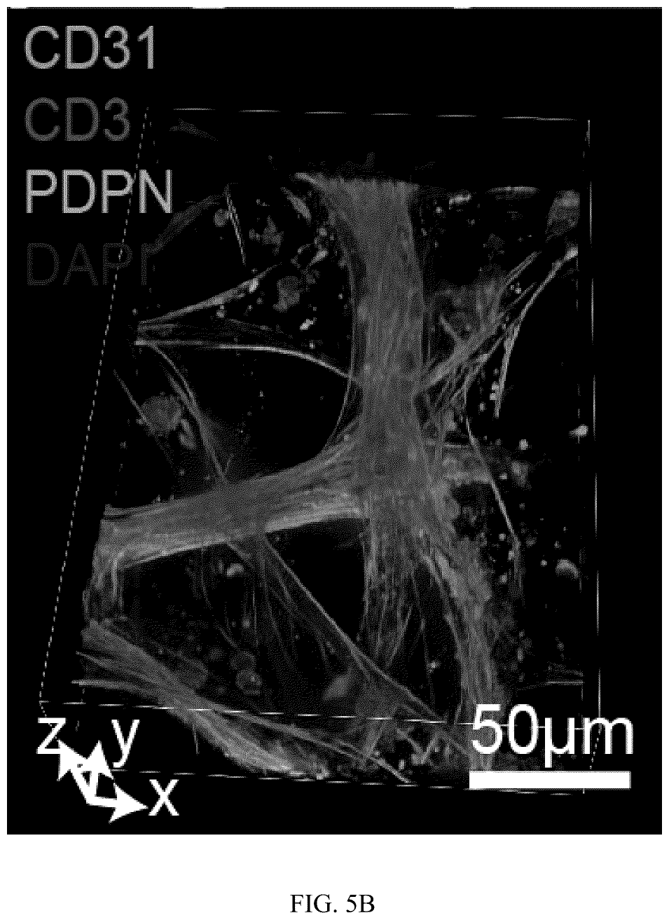

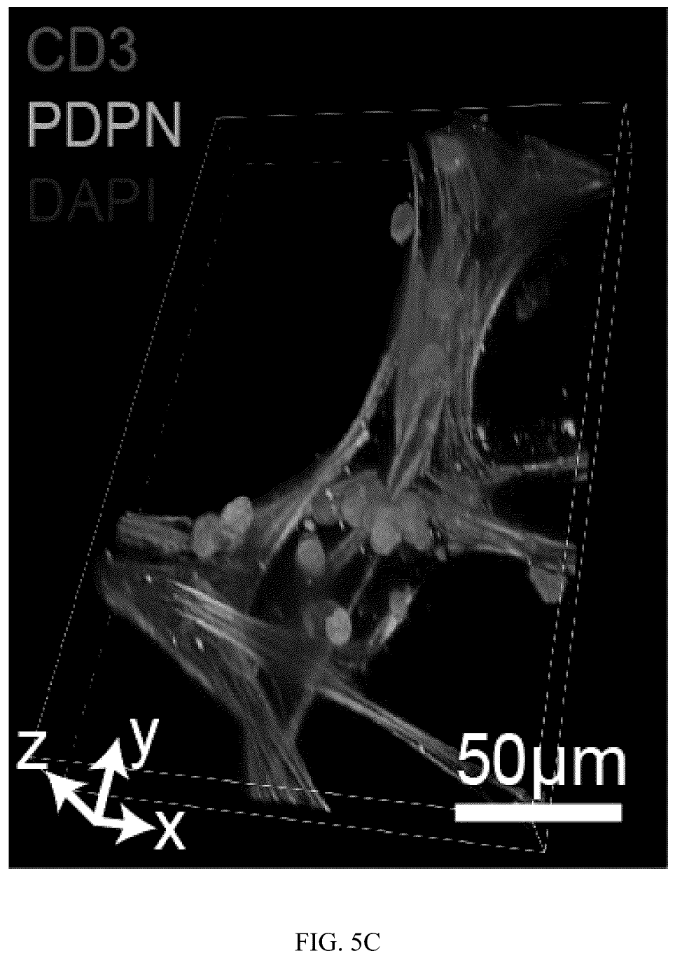

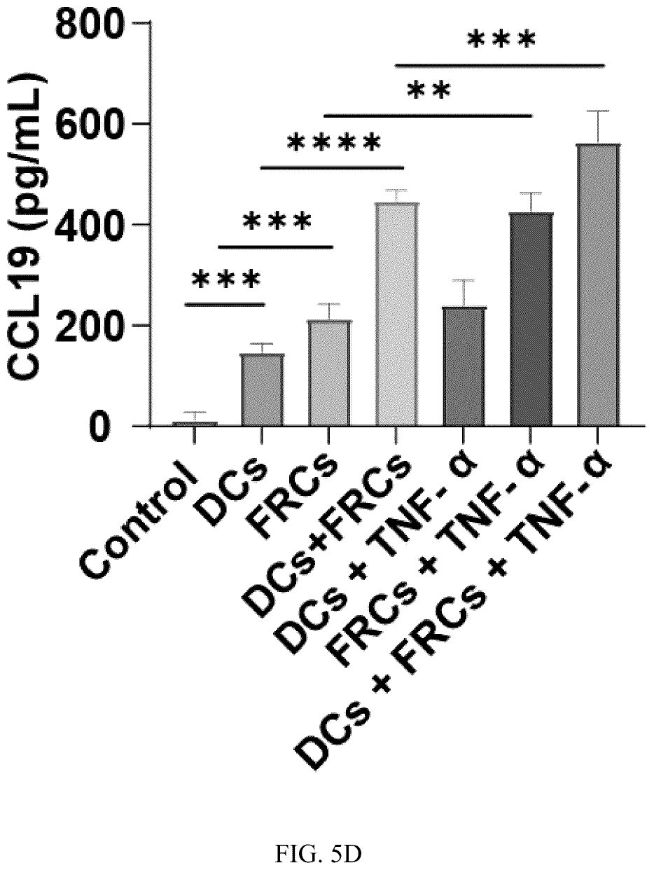

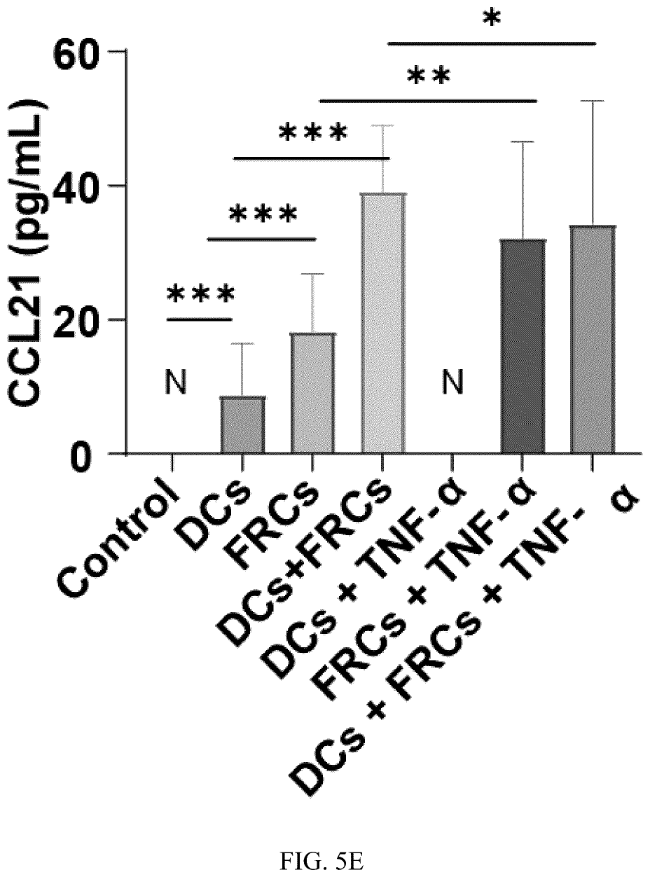

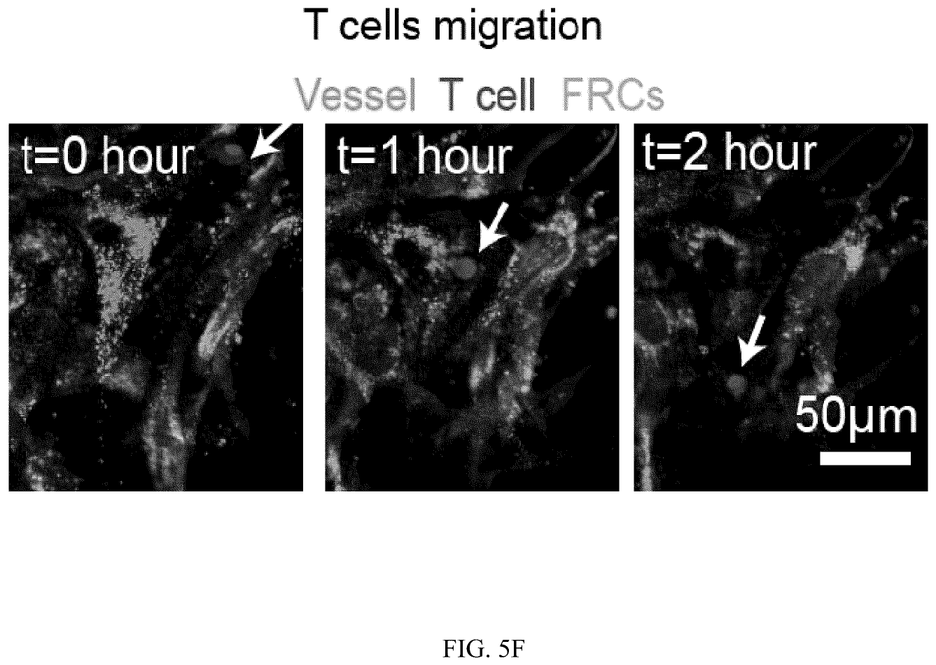

3D blood vessels (HEVs) facilitated the recruitment process of T cells and B cells in LN: Based on the disclosed results, 3D blood vessels were formed through self-assembly and grew into the T cell zone on the chip in 5-7 days. The area and diameter of formed vessels were measured under a confocal microscope (Nikon C2i) (see FIG. 5A), and the intactness of the blood vessels were confirmed by Dextran Alexa Fluor™ 488 (Thermo Fisher). Based on the disclosed results, it was shown that FRCs grew around blood vessels (see FIG. 5B), which verified the potential ability of FRCs to support the generation of HEVs and form a reticular system. FIG. 5A through FIG. 5F shows remodeling of the stromal niche of paracortical regions on the disclosed LN on a chip device. FIG. 5A are images showing HEVs and FRCs reticular system in the paracortical areas of the disclosed LN on a chip device. FIG. 5B shows perivascular FRC around blood vessels that support vessel integrity on the disclosed LN on a chip device. FIG. 5C shows T cells attach and grow on the FRCs surface. FIG. 5D and FIG. 5E are plots showing results of the secretion of CCL19 and CCL21 by DCs and FRCs in the paracortical area increases under TNF-α. FIG. 5F are images showing T cells migrate along the FRCs stromal reticulum on the disclosed LN on a chip device. In order to further understand the differences in the spatial distribution of inflammatory FRC, CD157 and gp38 were used to verify the spatial distribution of T cell region FRCs (gp38⁺, CD157⁺) and perivascular reticular cells (gp38⁻, CD157⁺) in the paracortex region. More perivascular FRCs surrounding CD31⁺ vessels in the T cell zone supports the intactness of the blood vessels [H. G. Alvarenga, L. Marti, J Immunol Res 2014, 402038 (2014)].

T cell region FRCs (TRCs) form a reticular system to facilitate lymph flow and T cell-DC interactions within the paracortex (see FIG. 4C) [T. Katakai et al., J Exp Med 200, 783-795 (2004)]. In the disclosed device, it was shown that in response to continuous contact with lymphocytes, FRCs secreted ER-TR7 antigen as an ECM component to form FRCs stromal reticulum within 5 days, a reticular meshwork of FRCs and ECM that supports the movement and interactions of DCs and T cells within the paracortex area (see FIG. 5C) [T. Katakai et al., J Exp Med 200, 783-795 (2004)]. Further, the disclosed device displayed that CCL19 and CCL21 released by mDCs and FRCs attracted T cell migration toward mDCs [W. H. Koh et al., iScience 23, 101427 (2020); K. Veerman et al., Cell Rep 26, 3116-3131.e3115 (2019)]. Based on the disclosed results, it was shown that under inflammatory conditions affected by TNF-α, the secretion of CCL19 and CCL21 by DCs and FRCs increased (see FIG. 5D & FIG. 5E), and it was expected that T cell migration would increase at this time. CD4⁺ T cells and FRCs pre-labeled with lipophilic trackers DiD and DiO were loaded into the system, and images were captured by confocal microscope (Nikon C2i) for 2 hours. This allowed for the observation of the T cells within the FRCs stromal reticulum. ImageJ was used to quantify the distance and speed of T cell migration. As shown in the results, the T cells migrated along the stromal network formed by FRCs (see FIG. 5F). Additionally, using the disclosed device and method allows for the examination of the effect of frequency of T cell-FRC contacts in the T cell region on the production of the ER-TR7 antigen using ELISA, deposition of laminin or fibronectin during the formation of the FRC stromal reticulum using immunostaining.

FDCs are located in B cell follicles, where they present antigen via complement receptors to B cells and form a dense network support primary B-follicles and germinal center formation (see FIG. 4B) [L. G. Barnett et al., J Immunol 192, 3607-3617 (2014)]. FDCs were stained with CD35 and CD21, and formed an FDCs stromal reticulum in the B follicle region after 10 days. Using immunostaining, it was shown that the formation of FDC stromal reticulum is accompanied by the deposition of collagen I and the attachment of a large number of CD19⁺ B cells. If the FDC stromal reticulum formation is not ideal, TNF-α (10-100 ng/mL) may be added to the chip to create an inflammatory environment within the simulated LN in a manner that promotes the formation of FDC stromal network [N. Li et al., J Immunol 178, 4214-4221 (2007); L. Gillot et al., Cell Mol Life Sci 78, 5987-6002 (2021)].

Sustainable perfusion of lymph fluid: Lymph fluid flow is vital for immune stability in LN. Lymph fluid flows in lymphatic vessels can assist in transporting antigens, activating immune cells, establishing chemokine gradients, and maintaining a dynamic environment required for effective immune responses [K. G. Birmingham et al., iScience 23, 101751 (2020); H. R. Hampton, T. Chtanova, Front Immunol 10, 1168 (2019); H. R. Hampton et al., Nat Commun 6, 7139 (2015)]. Engineered microfluidic channel simulations were used to simulate lymphatic vessels in a chip model, and simulate the entry/exit of lymph into/out of LN by loading media and antigens via an external peristaltic pump (Cole Parmer). This process was mimicked on the chip by designing the subcapsular sinus with a specialized layout of micropillars (see FIG. 3A). The flow rate of lymph fluid through human LNs is highly variable, ranging from a few microliters per hour to tens of microliters per hour. Actual rates vary based on factors like health, node location, and physiological conditions [D. C. Zawieja, Lymphat Res Biol 7, 87-96 (2009)]. The flow rate of the perfusion system was optimized from 0-120 μL/h to simulate lymph flow within the lymphatic system. Furthermore, the lactic acid secreted due to the enhanced metabolic capacity during T cell activation reduced the pH of the paracortex area in the LN and inhibited the immune function of T cells [H. Wu et al., Nat Commun 11, 4113 (2020)]. pH changes of the culture medium were monitored in the chip with or without flowing lymph and the changes in T cell activation-related cytokines (IL-2, IL-10, INF-γ) were compared through ELISA. Lymph fluid maintained the pH stability of the microenvironment in LN, and the efficiency of T cell activation was improved under dynamic culture conditions.

One goal of the disclosed device and method was to reconstitute the LN stromal niche in vitro. However, there were two potential challenges identified: First, when monitoring T cell migration along FRCs using confocal microscopy and lipophilic trackers DiD and DiO, there is the potential for fluorescence quenching during prolonged observation. To address this, a cell line, GFP-FDCs, was constructed that can stably express green fluorescent protein. This was achieved by transfecting the pLenti-CMV-GFP-Puro plasmid (Plasmid #17448) using a lentivirus. Second, there was some uncertainty regarding whether DCs can effectively enter the T cell area in the chip through continuously injected lymph fluid. This might be due to precipitation or attachment to the tube wall. To address these challenges, a solution was to mix the treated DCs with T cells and load them into the paracortex region.

Modeling adaptive immune processes to vaccine in the LN-on-a-Chip device: LNs play a pivotal role in human adaptive immune response that are vital for vaccine efficacy. After vaccination, vaccines drain to the LNs, where DCs recognize and phagocytize vaccine antigens, then activate naïve CD4⁺ T cells in the paracortex and induce naïve B cell activation and differentiation into memory B cells and plasma cells in the B follicles that sustain long-term immune responses (see FIG. 2B). Thus, the disclosed LN-on-a-Chip device simulates the key adaptive immune process in LNs (T cell activation, germinal center formation, and antibody secretion) in response to human seasonal influenza virus vaccine (ATCC BEI) and Covid-19 inactivated virus (ATCC BEI).

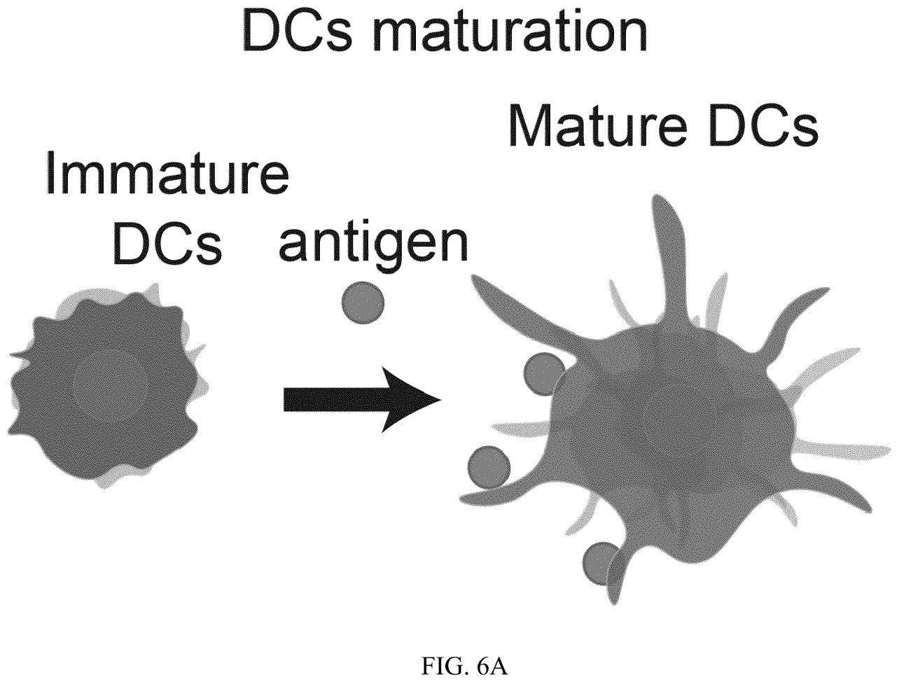

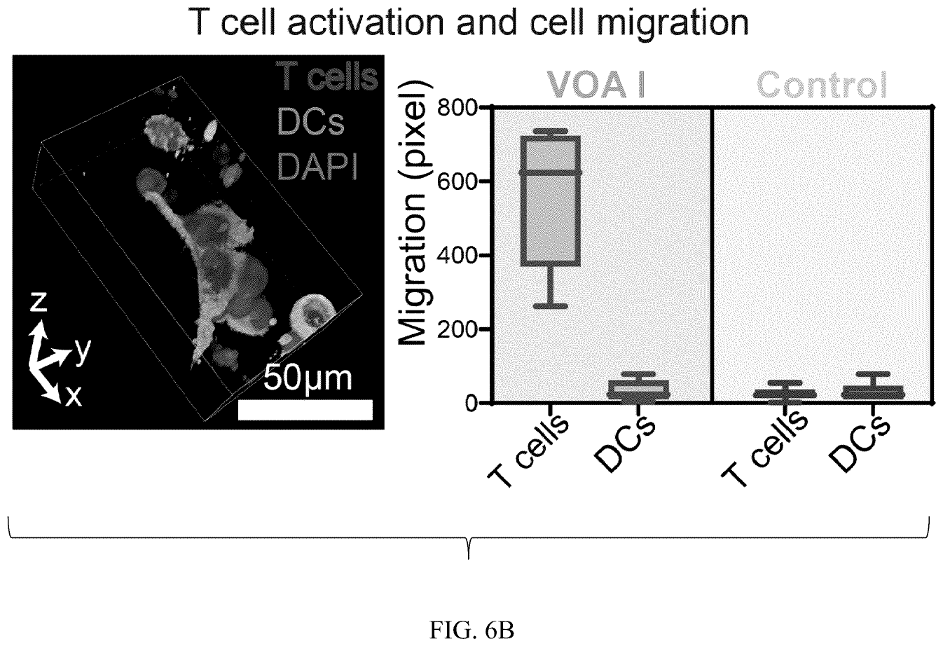

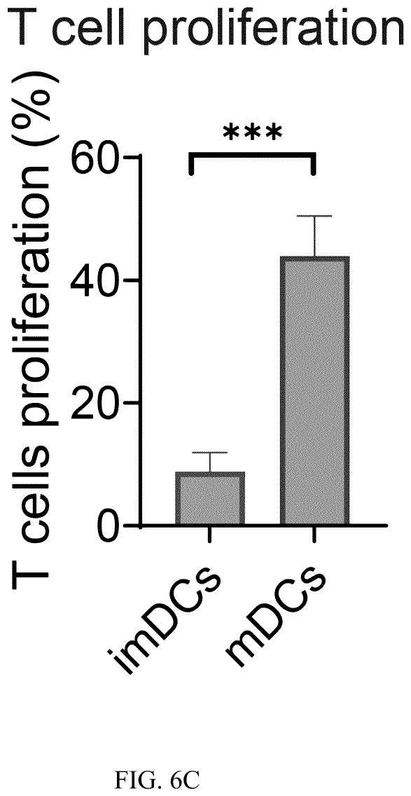

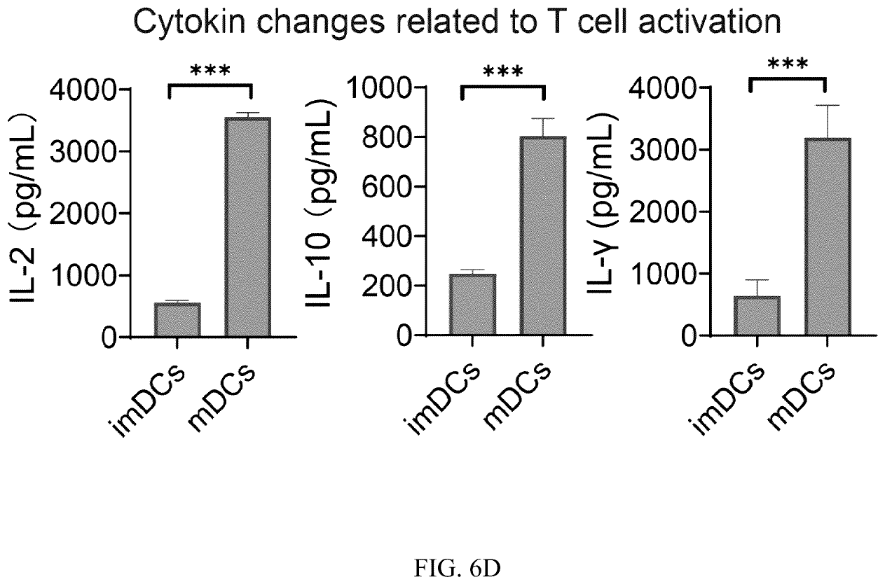

DC maturation and antigen presentation: After capturing the antigen, immature DCs (imDCs) transform into mature DCs (mDCs) (see FIG. 6A) and enter the paracortical area with lymph fluid (see FIG. 4A), where they contact and activate T cells (see FIG. 4C). This antigen presentation process marks the beginning of the adaptive immune response [C. Fu et al., Immunological Investigations 51, 2133-2158 (2022)]. Thus, using the disclosed device it was assessed whether vaccine antigens can induce imDC maturation and trigger adaptive immune response. In some embodiments, human seasonal influenza virus vaccine was used to induce DC maturation on or off chip for 7 days, then flow cytometry was used to analyze the maturation efficiency and phenotype of DCs with a panel of surface markers for imDCs (CD11c, CD1a) and mDCs (CD40, CD80, CD83, and HLA-DR). It was shown that upon vaccine antigen stimulation, the expression of mDC markers CD40, CD80, CD83, and HLA-DR increased significantly. In the disclosed example, Ovalbimin (OVA I, Sigma Aldrich) was used as model antigen [S. H. Karandikar et al., JCI Insight 5, (2019); J. Blobner et al., Neuro-oncology Advances 3, vdab147 (2021)] to initiate the adaptive immune process in the LN chip, and it was observed that imDCs transformed into mDCs, thus resulting in enhanced T cell migration, proliferation and activation (see FIG. 5A through FIG. 5F).

FIG. 6A through FIG. 6D depicts a DC-mediated T cell activation on the disclosed LN on a chip device. FIG. 6A depicts the maturation of DCs. FIG. 6B shows imaging and results of the effects of OVA I-induced mDCs and imDCs (control) on T cell migration in the paracortex area on disclosed LN on a chip device. FIG. 6C is a plot showing results indicating that T cell proliferation is higher on chips with mDCs than chips with imDCs. FIG. 6D are plots showing results indicating the secretion of T cell activation-related cytokines increased in chips with mDCs than chips with imDCs.

T cell migration and activation stimulated by mDCs and FRCs (T cell Zone): Successful antigen presentation by DCs will increase T cell migration in LNs. CCL19 and CCL21 released by mDCs and FRCs will attract T cell migration toward mDCs [W. H. Koh et al., iScience 23, 101427 (2020); K. Veerman et al., Cell Rep 26, 3116-3131.e3115 (2019)]. First, the concentration changes of CCL19 and CCL21 in the culture medium by ELISA were determined, after constructing the FRC stromal reticulum, and after loading mDCs respectively. Second, the enhanced T cell migration in the paracortex area influenced by mDCs and FRCs was verified. The migration of DCs and CD4⁺ T cells pre-labeled with CellTracker Red and DID dyes were tracked under a fluorescence microscopy for 1-3 days under treatment with human seasonal influenza virus vaccine. DC and T cell migration trajectory and speed were quantified in the paracortical area using Image J. Based on the disclosed results, vaccine-induced mDCs resulted in stronger T cell motility in LN (see FIG. 5B). Further, T cell migration along the FRC stromal reticulum toward antigen-carrying mDCs in paracortex area was observed. With control experiments it was proven that without mDCs and/or FRC stromal reticulum, T cell motility in paracortex area was significantly hindered. It was also observed that CD4⁺ T cells were activated after mDCs entered the paracortical area. T cell prelibation rate was measured (see FIG. 6C) using EdU (Sigma Aldrich) and changes in key cytokines related to T cell activation (IL-2, IL-10, INF-γ) were detected using ELISA (see FIG. 6D) over 7 days. Additionally, the ratio of two primary T cell subtypes in LNs, CD4⁺ helper T cells and CD8⁺ cytotoxic T cells, were analyzed upon mDC simulation [S. Poppema et al., J Exp Med 153, 30-41 (1981)]. Flow cytometry was used to analyze CD4⁺ T cell subsets (Th1, Th2, Th17, Treg cells) after T cell activation.

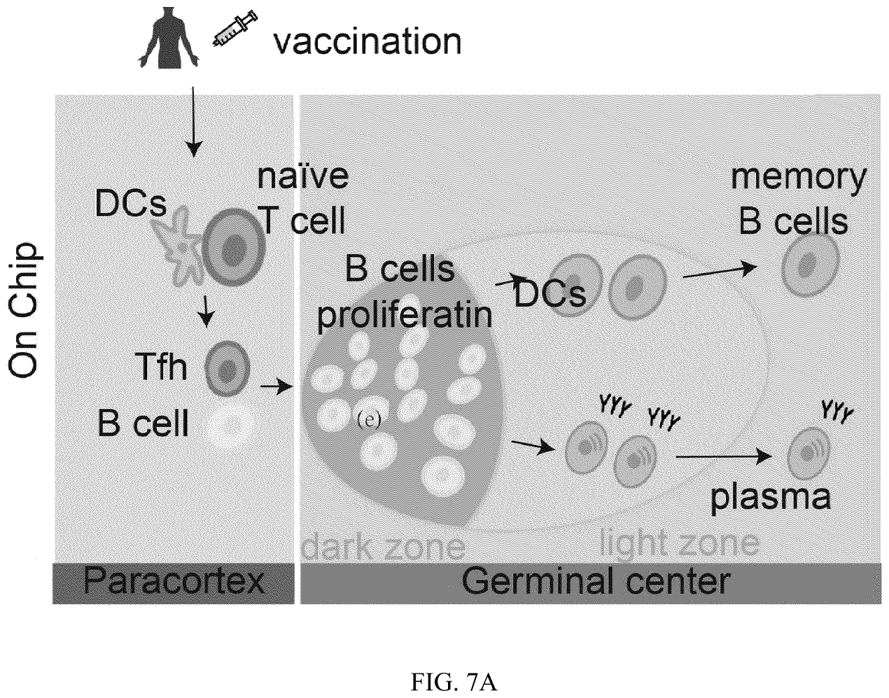

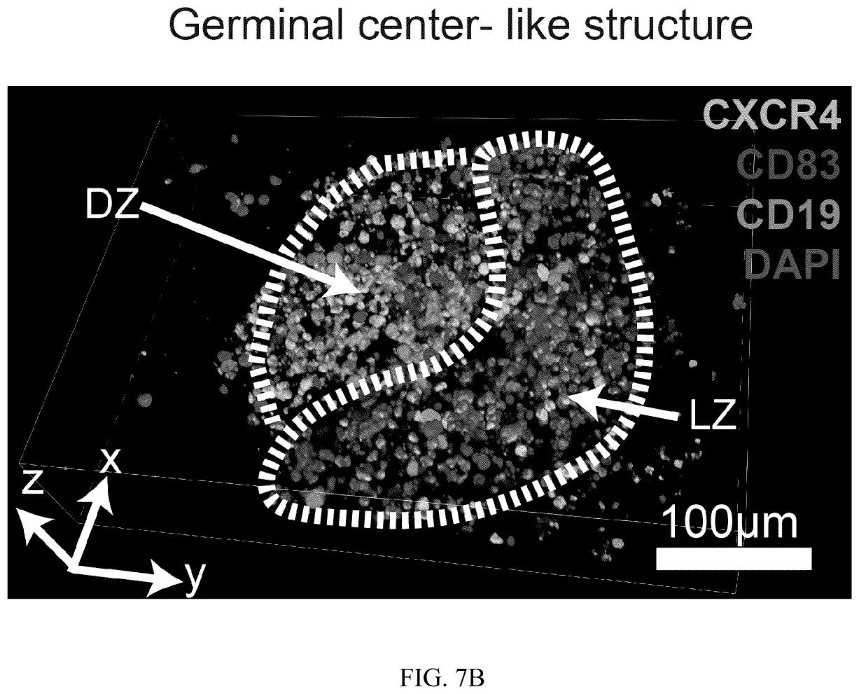

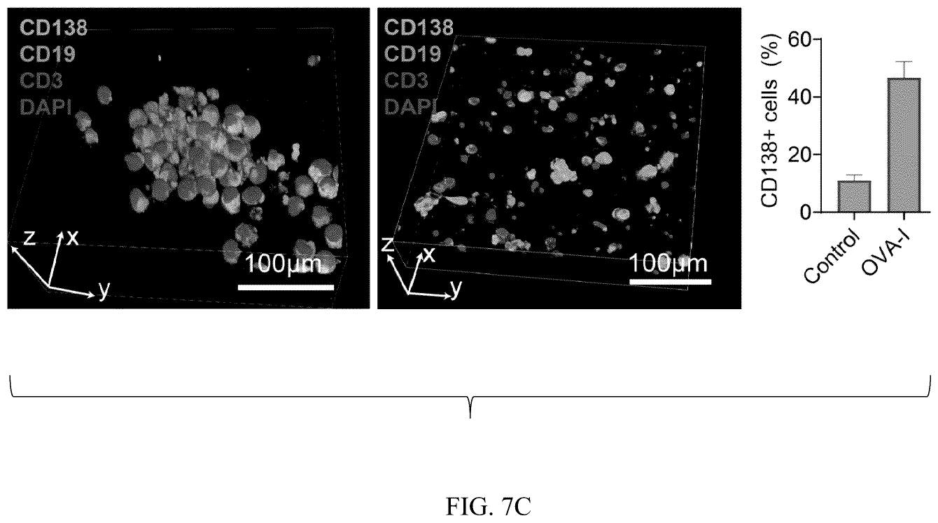

B cell differentiation and germinal center formation (B cell follicle zone): A key feature of adaptive immune response is the formation of germinal centers in B cell follicles [C. Young, R. Brink, Immunity 54, 1652-1664 (2021)]. In this process, naïve B cells, with the assistance of regulatory T cells, differentiate into memory B cells capable of producing antigen-specific antibodies to sustain long-term immunity (see FIG. 7A). The formations of a large number of germinal center-like structures in the B cell follicles region 10 days after mDCs activate T cells was shown. GL7-expressing B cells were stained to label germinal center structures, and cells with CD83 and CXCR4 were stained to define light and dark areas of the germinal center structure (see FIG. 7B) [L. E. Wagar et al., Nat Med 27, 125-135 (2021)]. The size and spatial distribution of germinal centers within the B follicle were quantified by Image J. The differentiation of B cells into CD27⁺ memory B cells and CD138⁺ plasma cells were verified using immunostaining (see FIG. 7C). The subtypes of B cells in the B follicles using flow cytometry were quantified as per the following panel: naïve B cells (CD27⁻CD38⁻), pre-germinal center (GC) B cells (CD27⁻CD38⁺), GC B cells (CD27⁺CD38⁻), memory B cells (CD27⁺CD38⁻), plasma cells (CD27⁺CD38⁺⁺) [L. E. Wagar et al., Nat Med 27, 125-135 (2021)]. The secretion levels of cytokines related to B cell activation were measured, such as IL-4, IL-6, and IL-7, in the conditioned medium using ELISA. The proliferation (Ki67) rates of T cells and B cells on chip were also quantified to confirm the activation of adaptive immune responses upon the vaccine antigen stimulation.

FIG. 7A through FIG. 7C shows B cell differentiation and germinal center formation on the disclosed LN on a chip device. FIG. 7A is a diagram showing a paracortex region and formation of germinal center structure. FIG. 7B is a staining image of germinal center structure formed in B cell follicles on chip upon antigen stimulation. FIG. 7C are staining images and quantitative results showing CD138+ plasma cells in B cell follicles with (left) and without (right) antigen stimulation.

Further, the impact of FDCs on germinal center formation was explored. CD40L-expressing FDCs form a dense network in the B cell follicles and are known to contribute to the formation of germinal centers [L. G. Barnett et al., J Immunol 192, 3607-3617 (2014)]. Thus, B cell differentiation and generation center formation was validated with/without FDCs. It was anticipated that in the absence of FDCs, B cell differentiation and germinal center formation would be impeded. Due to the scarcity of human-derived FDC, alternatively it was proposed replacing FDCs with supplementing CD40L and IL-4 in the culture medium [S. G. Tangye et al., J Immunol 169, 4298-4306 (2002)] to promote the formation of germinal center structures. Moreover, germinal centers only form when both T and B cells successfully interact at the T-B border and migrate within the B-cell follicle containing the FDC network. CD4⁺ helper T cells 10 days after mDCs activated T cells were stained, in order to prove that FDCs secrete the chemokine receptor CXCL13, and guide CD4⁺ helper T cells into the T-B border region and interact with CD19⁺ B cells thus support formation of germinal centers [J. Kranich, N. J. Krautler, Front Immunol 7, 225 (2016)].

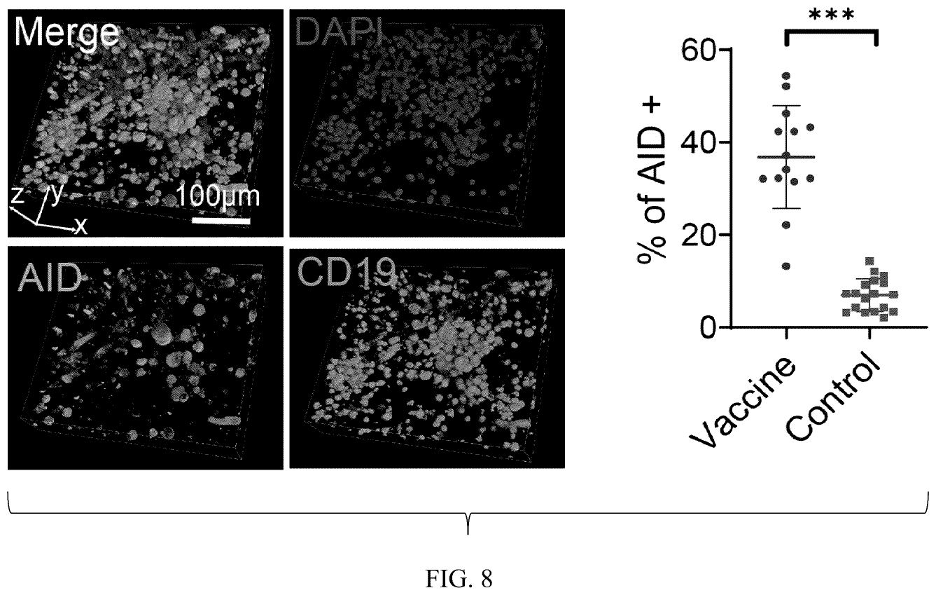

Antibody secretion: Secretion of antibodies is an important sign of the adaptive immune response and the effectiveness of vaccine. Therefore, secretion levels of total IgG, IgM, and vaccine-specific neutralizing antibodies (Influenza A antibody, Influenza B Antibody, Novus Biologicals) were measured over one week from the chip by ELISA after vaccination. A significant increase in secretion levels of these antibodies was shown on chip upon vaccine stimulation. Moreover, DNA-modifying enzymes are required by B cells to switch from one antibody isotype to another. For instance, Activation-Induced Cytidine Deaminase (AID), are exclusively expressed by B cells residing within lymphoid follicles, but they are not expressed by B cells in peripheral blood [D. Mechtcheriakova et al., Cancer Immunol Immunother 61, 1591-1598 (2012)]. Thus, a significant increase in AID expression on B cells in the disclosed LN chip model 10 days after vaccine stimulation was shown (see FIG. 8). FIG. 8 is an image and plot showing results of AID expression of B cells derived from peripheral blood in the disclosed LN on the chip.

Gene expression (scRNAseq) analysis. As adaptive immune response proceeds, DC maturation (imDC to mDC), T cell differentiation (cytotoxic, helper, regulatory T cells), and B cell differentiation (memory B cells, plasma cells) occur in LNs. Thus, cells were retrieved from the LN chips and processed with scRNAseq analysis [W. Stephenson et al., Nat Commun 9, 791 (2018)]. Variable cellular states and subsets of immune cells were stratified using the single-cell transcriptome sequencing data, enabling a detailed understanding of cellular heterogeneity.

Multidimensional evaluation of vaccine effectiveness. Based on the disclosed example, five key functional indices normalized with range of 0-1 are provided to comprehensively evaluate vaccine effectiveness. 1. Immune cell phenotypes: DC maturation (CD80, CD83, and HLA-DR), T cell proliferation (ki-67) and differentiation types (CD4⁺ Th cells, CD8⁺ cytotoxic T cells), B cell differentiation types (CD27⁺ memory B cells, CD138⁺ plasma cells); 2. Lymphocyte trafficking: DC recruitment and T cell migration; 3. Proteomic characterization related to immune cell homing (CCL19, CCL21), T cell activation (IFN-γ, IL-10, IL-8, IL-2), T cell migration (e.g. CCL17), and B cell differentiation (IL-4, IL-6); 4. Number and size of germinal centers (GL7⁺); 5. Antibody secretions: total IgG, IgM, and vaccine-specific neutralizing antibody concentrations.

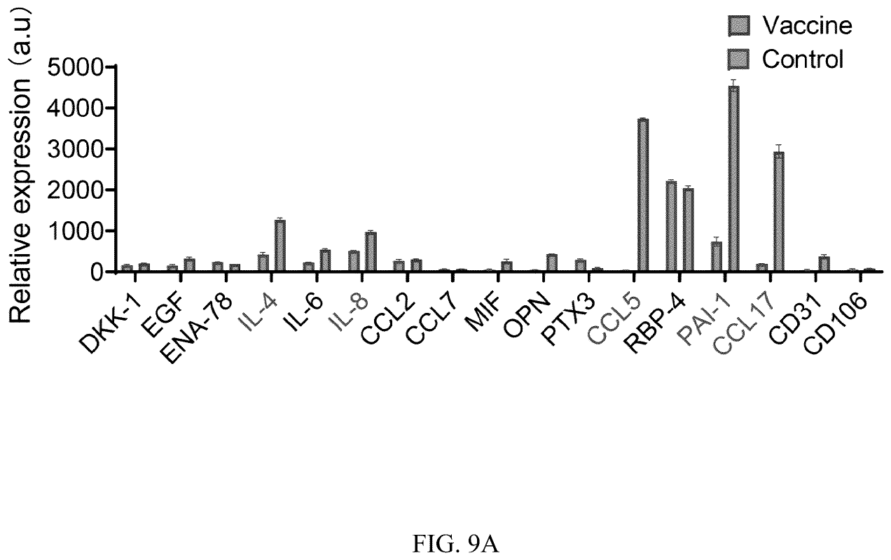

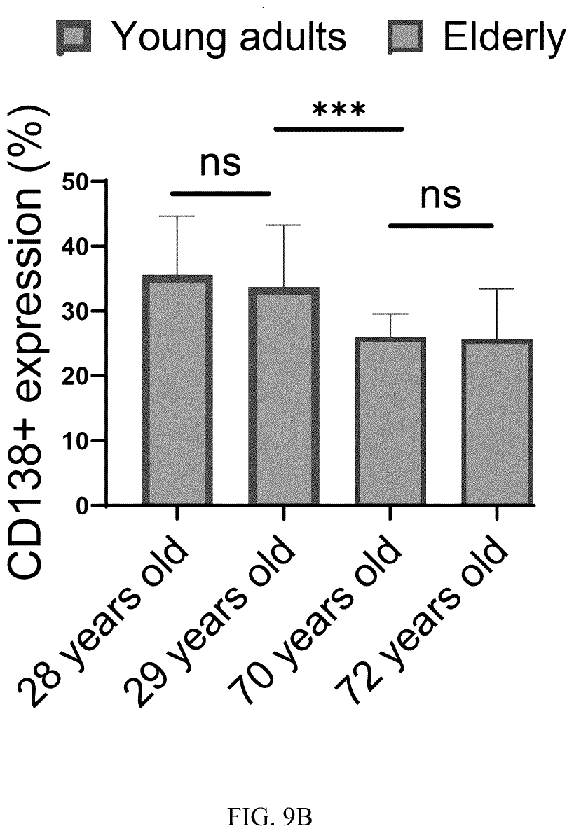

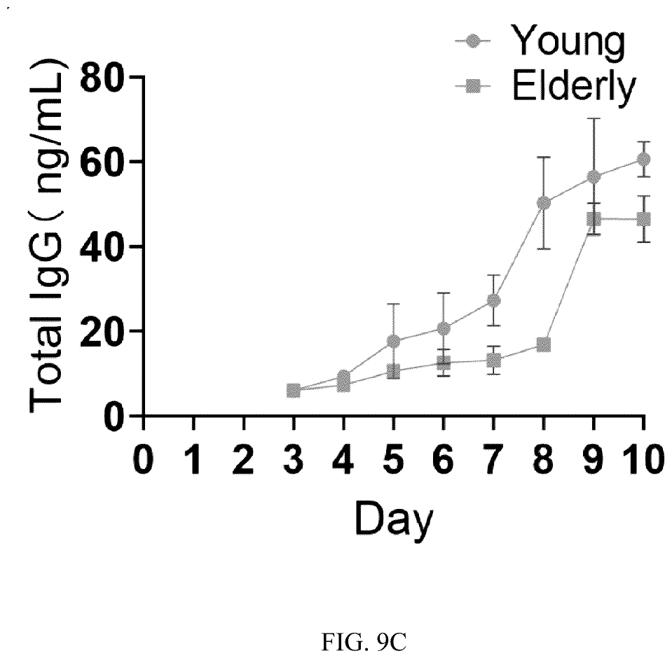

Evaluation of vaccination effectiveness in different population: The goal was to develop a robust and reproducible LN-on-a-chip platform (i.e., LN on a chip device) for large-scale vaccine efficacy assessment in diverse populations, including different age groups, health statuses, genders, and more. In the disclosed example, the LN chip was loaded with immune cells both from young (age: 28 and 29 years) and old (age: 70 and 72 years) and the population groups were validated showing distinct immune responses (see FIG. 9A through FIG. 9C). Based the disclosed example, it was shown that older age groups exhibited a less robust response to the vaccine. In some embodiments, the disclosed LN-on-a-Chip device may be applied to various populations, including healthy individuals and those with chronic diseases such as diabetes, men and women, among others. The goal was to create a robust and reproducible LN-on-a-Chip device for assessing vaccines in diverse populations. These different factors such as age, gender, race, health status may be explored with the disclosed device, and will elucidate adaptive immune processes within LNs and thus the efficacy of vaccines. In some embodiments, the disclosed LN-on-a-chip device may be used to assess different types of vaccines and vaccination strategies, such as mRNA therapies, and combined therapies.

FIG. 9A through FIG. 9C are plots showing results for the evaluation of seasonal influenza virus vaccine on the disclosed LN on a chip device using cells sampled from different subject age groups. FIG. 9A is a plot showing the results of the changes in key cytokines/chemokines of adaptive immunity. FIG. 9B is a plot showing the results of cell differentiated CD138+ plasma cells. FIG. 9C is a plot showing the results of Total IgG secretion.

Impact of vaccine adjuvants and vaccination strategies on vaccine efficacy: the impact of common vaccine adjuvants, such as aluminum salts (Strem Chemicals) and MH59 (InvivoGen) were investigated on vaccine effectiveness [T. Zhao et al., Signal Transduct Target Ther 8, 283 (2023)]. It was expected that vaccination effectiveness would increase with the addition of vaccine adjuvants. In some embodiments, using the disclosed device the impact of different adjuvants on vaccination may be evaluated according to the above vaccine effect evaluation method. At the same time, the number of vaccinations and vaccination concentration may be adjusted to investigate the optimal vaccination strategy in different populations. Then, the best vaccination strategy may be chosen according to the vaccine evaluation method mentioned above. Ultimately, the goal of the disclosed device and method is to provide a valuable tool for vaccine development and the assessment of vaccination strategies. The disclosed LN-on-a-chip device helps to discover optimized vaccination strategies for different population groups by combining vaccine adjuvants.

An in vitro model of LN that is reproducible, easily accessible, and perfectly reproduces different populations was generated in the disclosed example. This technology allows testing of vaccines and adjuvants on long-term adaptive immune responses from various populations, considering factors like age, gender, race, health status, and genetic variations, and thus aids in optimizing vaccination strategies for optimal protection for different populations. Age and chronic disease-related changes in LN niches aren't solely attributed to variations in immune cells. Alterations in stromal cells, such as fibroblasts, and the development of ECM fibrosis can also impact the adaptive immune response within LN. To account for these factors, in some embodiments, appropriate cell sources, such as FRCs in the elderly population, people with high blood pressure and diabetes are sourced for the LN-on-a-chip device. Importantly, the fundamental structure of the model does not need to be modified. Instead, these new cell sources are incorporated into the existing framework to comprehensively address these aspects and gain a more holistic understanding of adaptive immunity in diverse populations.

Statistical Analysis and Rigor: All chip experiments used 12 devices with 4 repeats per condition in parallel with appropriate control groups to ensure reproducibility. The large number of replicates possible illustrates a clear strength of the device and method. Data was compared between groups with and without antigen stimulation. T cell activation was assessed, including changes in cytokine (IFN-γ, IL-10, IL-2) concentrations, cell subpopulations (CD4⁺ Th cells, CD8⁺ cytotoxic T cells), and average migration speed. Additionally, B cell differentiation (CD27⁺ memory B cells, CD138⁺ plasma cells) was examined, focusing on changes in cytokine (IL-4, IL-6) concentrations and cell subpopulations. Germinal center formation (GL7⁺) was also analyzed in terms of the number and density of these centers. Furthermore, antibody secretion was investigated, including IgG and IgM differences. Factors associated with adaptive immunity were compared within 4 to 10 days of vaccine antigen stimulation. Specifically, the activation status of lymphocytes, the formation and proliferation of germinal centers, and the secretion of relevant molecules involved in the adaptive immune process was assessed. Markers indicative of vaccine effectiveness include T cell proliferation (Ki67 expression), the development and expansion of germinal centers (measured by GL7 and Ki67 expression), and the secretion of specific molecules related to the adaptive immune response. Continuous variables were presented as means and standard deviations, while categorical variables were expressed as counts and percentages. The normality of all data was assessed using the Shapiro-Wilk and Shapiro-Francia tests. To determine statistical significance, the Mann-Whitney U and Kruskal-Wallis tests for continuous variables, and the chi-square or Fisher's exact test for categorical variables were utilized, as appropriate. Statistical significance was defined by a two-sided p-value of p<0.05.

Other applications: Lymphoma: The disclosed LN chip device effectively replicates the complex microenvironment of lymph nodes (LNs), critical sites for the development and evolution of lymphoma. Importantly, the disclosed chip does not require structural changes. It can be used to simulate B-cell lymphoma simply by changing the cell types, such as introducing B-cell lymphoma cells. By simulating the physiological conditions of LNs, the LN Chip enables researchers to delve deeper into the mechanisms of lymphoma's initiation, growth, and spread within a highly realistic lymphatic context. The LN Chip is instrumental in elucidating the complex interactions between lymphoma cells and the immune system. Understanding these dynamics is essential for the advancement of novel immunotherapeutic strategies, which aim to harness and enhance the body's immune response against cancer cells. The chip's ability to mimic these interactions can reveal new pathways and targets for intervention, potentially leading to breakthroughs in cancer immunotherapy. Another remarkable feature of the LN Chip is its potential for personalization. By incorporating cells derived from individual lymphoma patients, the chip can simulate the specific characteristics of a patient's cancer, paving the way for personalized medicine approaches. This individualized strategy could revolutionize treatment protocols, allowing clinicians to tailor therapies based on the unique genetic and molecular profile of each patient's lymphoma, thus maximizing efficacy and minimizing side effects.

Lymph node metastasis: Cancer cells break away from the primary tumor, travel through the lymphatic system, and establish new tumors in LNs. The presence of tumor cells in the LN is often a critical factor in cancer staging, which helps determine the prognosis and treatment strategy. The disclosed chip can be easily combined with other tumor chips to study in detail how cancer cells metastasize to lymph nodes and the subsequent immune response. This is particularly significant in understanding and combating cancer spread. Furthermore, the disclosed LN chip has potential value in specific antigen screening. By identifying unique proteins or antigens present on tumor cells, it aids in understanding the immune system's recognition and response mechanisms, which are central to developing targeted immunotherapies like cancer vaccines or adoptive T cell therapies. Additionally, the adaptability of the disclosed chip to use patient-specific cells marks a significant stride towards personalized medicine, enabling the modeling of individual tumor-immune interactions and facilitating the testing of tailored treatment strategies. This personalized approach is instrumental in determining the most effective antigens and therapies for each patient.

Studies on LN associated viral infectivity are crucial for understanding the complex interactions between viruses and the immune system. LN, integral components of the immune system, can act as sites for virus replication and storage, where some viruses, like HIV, may persist in either an active or latent state. Viruses can modulate the immune response in LN, either by directly infecting immune cells or by altering the LN environment. This can impact the effectiveness of the immune response against the virus. At the start of the AIDS epidemic, lymphadenopathy was one of the first identified symptoms of HIV infection. Follicular structural changes caused by lymphadenopathy were later used to classify HIV progression into four stages: 1) follicular hyperplasia, 2) follicular lysis, 3) follicular atrophy, 4) follicular/lymphocytic depletion. In this final stage, CD4+ T cells are progressively depleted, leading to AIDS [E. M. B. Scholz, A. D. M. Kashuba, Clin Pharmacol Ther 109, 918-927 (2021)]. In the disclosed example, an in vitro lymph node (LN) model was constructed to simulate the human LN niche. The disclosed chip can replicate the complex environment of a lymph node, including its cellular and structural characteristics. This LN model holds promise for enabling studies of HIV's interaction with the immune system in a setting that is more physiologically relevant than traditional cell cultures. Antiretroviral therapy is currently a crucial method in treating AIDS, but its effectiveness varies among individuals. The disclosed LN chip can monitor cell types and matrix changes in the LN in real-time, making it an innovative platform for drug testing that could facilitate the development of more effective antiretroviral treatments. Additionally, the disclosed LN chip has the potential to simulate individual immune responses, leading to personalized medical approaches. The disclosed LN chip bridges the gap between traditional cell culture and animal models. This technology holds significant potential for HIV research, providing new insights into the complex interactions between HIV and the human immune system.

### Example 2: Lymph Node-on a Chip: Modeling Human Adaptive Immune Responses to Assess Vaccine Efficacy

Vaccines play an essential role in controlling and preventing infectious diseases in humans [Jeyanathan M et al., Nat Rev Immunol 2020; 20(10):615-32]. However, Vaccine development is an exceedingly intricate and time-consuming process [Buckland B C, Nat Med 2005; 11(4 Suppl):S16-S9]. The lack of comprehensive preclinical data and a dearth of precise information on the correlates of immune protection have frequently led to vaccine products failing in clinical trials [Pulendran B, Davis M M, Science 2020; 369(6511)]. To address this issue, it is crucial to develop more relevant models and to collect and analyze human samples extensively. However, interspecies differences between animal models and humans hinder accurate replication of immune responses following vaccination [Walls A C et al., Cell Rep 2022; 40(9):111299]. Furthermore, ethical and safety concerns surrounding paid recruitment of clinical volunteers have faced widespread criticism [Calina D et al., Daru 2020; 28(2):807-12]. These underscores the pressing importance of devising new vaccine evaluation models. The efficacy of vaccine depends on the extent to which the vaccine enhances the adaptive immune response in the body, since vaccine antigens primarily activate the adaptive immune system, which remembers and eliminates the antigen as a foreign invader [Leleux J, Atalis A, Roy K, J Control Release 2015; 219:610-21]. Adaptive immune response is mainly initiated in lymph nodes (LNs), which serve as vital hubs for adaptive immune-related lymphocytes, including antigen-presenting cells (APCs), CD4⁺ helper T cells, CD8⁺ T cells, and B cells (see FIG. 2A & FIG. 2B) [Grant S M et al., J Cell Sci 2020; 133(5)]. However, changes in the lymph node (LN) niche such as depletion of CD4⁺ T cells, reduced germinal centers, and LN extracellular matrix (ECM) fibrosis due to genetic variation, disease, aging, or chronic inflammation can lead to delayed immune responses affecting vaccine efficacy across populations [Cakala-Jakimowicz M et al., Cells 2021; 10(11); Ahmadi O et al., ANZ J Surg 2013; 83(9):612-8; Chen J et al., Trends Mol Med 2022; 28(12):1100-11]. Therefore, understanding the impact of the LN niche on adaptive immune responses is critical for developing effective vaccines and new vaccination strategies tailored to different populations. FIG. 2A is a schematic diagram depicting the structure and function of human lymph nodes. FIG. 2B is a schematic diagram of the adaptive immune process in LN after vaccination.

Current challenges in human LN in vitro models and adaptive immunity research: LNs are highly structured secondary lymphoid organs with essential functions reliant on the unique spatial arrangement of lymphocytes and stromal cells, as well as the chemokines that drive the signaling cascades underlying the immune response [Liao S, von der Weid P Y, Semin Cell Dev Biol 2015; 38:83-9]. LNs are composed of various cell types, including T cells (CD4⁺ Helper T cells, CD8⁺ cytotoxic T cells), B cells (memory B cells, plasma cells), dendritic cells (DCs), fibroblast reticular cells (FRCs), follicular dendritic cells (FDCs), lymphatic and vascular endothelial cells [Fletcher A L, Acton S E, Knoblich K, Nat Rev Immunol 2015; 15(6):350-61]. Developing models of LNs that can accurately replicate the dynamics and organization of the intact LN niche, remains a significant challenge in human adaptive immunity research. No reliable in vitro model of LNs has been able to simulate the complete dynamic process of adaptive immunity [Ozulumba T et al., Front Immunol 2023; 14:1183286]. While humanized animal models are available for vaccine evaluation, they have limitations such as inefficient T cell and B cell cooperation, reduced immunoglobulin class switching efficiency, and a limited number of LNs [Herati R S, Wherry E J, Cold Spring Harb Perspect Biol 2018; 10(4)]. Additionally, 3D organoid cultures partially replicate organ complexity but struggle to model lymphatic fluid dynamics and vascular structure, which are essential for immune cell entry into LNs and adaptive responses. The lack of anatomical and microphysiological control in current organoid models leads to less controllable and reproducible experiments [Purwada A et al., Biomaterials 2015; 63:24-34]. Recently developed microfluidic chips have shown potentials for in vitro study of LN function, however, these models lack full LN niche and only focus on a partial function of LNs, such as lymphocyte migration or homing influenced by lymph fluid [Ozulumba T et al., Front Immunol 2023; 14:1183286]. More importantly, the scarcity of human LN samples largely limits the development of in vitro models of LNs [Kwiatkowska K M, Mkindi C G, Nielsen C M, Front Immunol 2022; 13:1045529]. Consequently, most current in vitro LN models use mouse [Stem P L, Ann Allergy Asthma Immunol 2020; 125(1):17-27] or immortal cell lines (such as Jurkat and Raji) [Sonmez U M et al., Micromachines 2020; 11(4); Chen X Y, Butt A M, Mohd Amin M C I, J Control Release 2019; 311-312:50-64], which cannot fully reflect the human adaptive immune responses. Recent studies suggest that blood-derived lymphocytes have potential to build up in vitro models of adaptive immunity [Goyal G et al., Adv Sci (Weinh) 2022; 9(14):e2103241; Westdorp H et al., J Immunother Cancer 2019; 7(1):302]. Under inflammatory conditions, peripheral blood mononuclear cells (PBMCs) can ectopically form lymphoid follicles, usually found within LNs, and have shown adaptive immune responses [Cabrita R et al., Nature 2020; 577(7791):561-5]. Moreover, high-density culture of PBMC-derived lymphocytes demonstrates antibody class switching and generation of plasma cell clusters after being stimulated by exogenous substances, similar to the process of germinal center formation during adaptive immune response in LNs [Römer P S et al., Blood 2011; 118(26):6772-82]. This led to the exploration of using human PBMC-derived lymphocytes, which are easily available on a large scale, to build an immunocompetent in vitro LN model.

In the disclosed example, an organotypic and immunocompetent human ‘LN-on-a-Chip’ microphysiological system (i.e., LN on a chip device) was developed to replicate the intricate structure and immunological function of natural LNs. Modeling and decoding the LN microenvironment using the disclosed LN on a chip device provides a new approach to model human adaptive immune response and offers a powerful tool for vaccine evaluation, screening, and new vaccination strategy development.

Primarily, a 3D immunocompetent in vitro LN-on-a-Chip model for the disclosed device was engineered using state-of-the-art microfluidic organ-on-a-chip technology to create a physiologically relevant LN microenvironment. Secondly, key adaptive immune processes were modelled to assess vaccine efficacy in the LN-on-a-Chip model. Ultimately, the goal was to develop an LN-on-a-Chip model that can be used to evaluate the effectiveness of vaccines and vaccination strategies in different populations (e.g., age, health status, gender, etc.).

The disclosed tissue engineered ‘LN-on-a-Chip’ microphysiological system (i.e., LN on a chip device) recapitulates the in vivo physiology of the human LN microenvironment, and simulates human adaptive immune processes in the LNs, thus aiding in evaluating vaccine effectiveness and optimizing vaccination strategies. In some embodiments, human PBMC-derived lymphocytes can be used to build an immunocompetent in vitro LN model to simulate human adaptive immune responses. The disclosed example validates that in certain subjects, adaptive immune response is hindered or delayed and thus vaccine effectiveness is reduced in elderly population due to T lymphocyte depletion, reduction of germinal centers, functional division confusion, and ECM fibrosis in LNs.

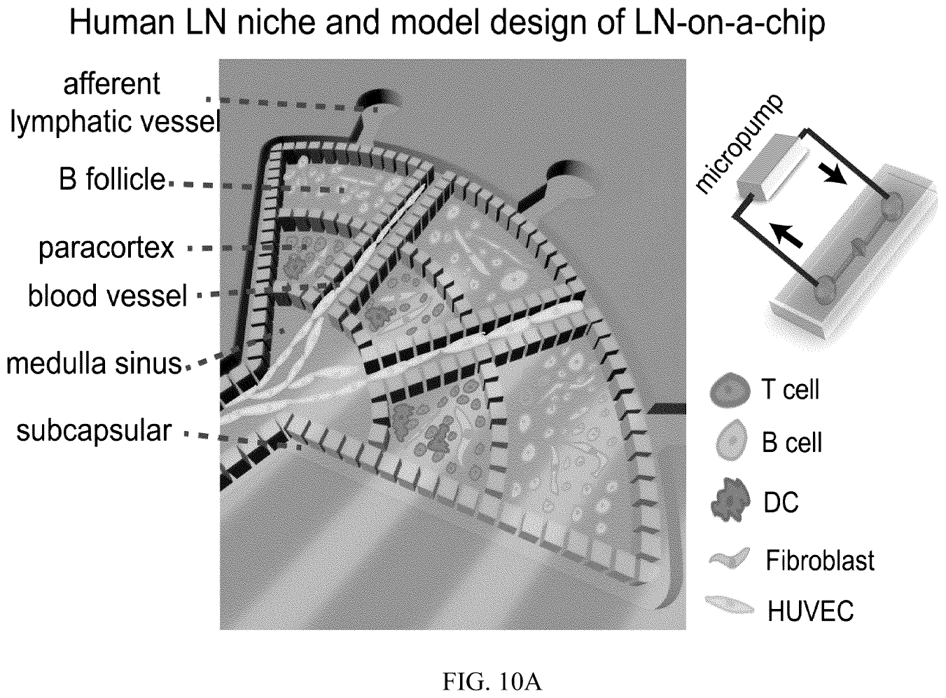

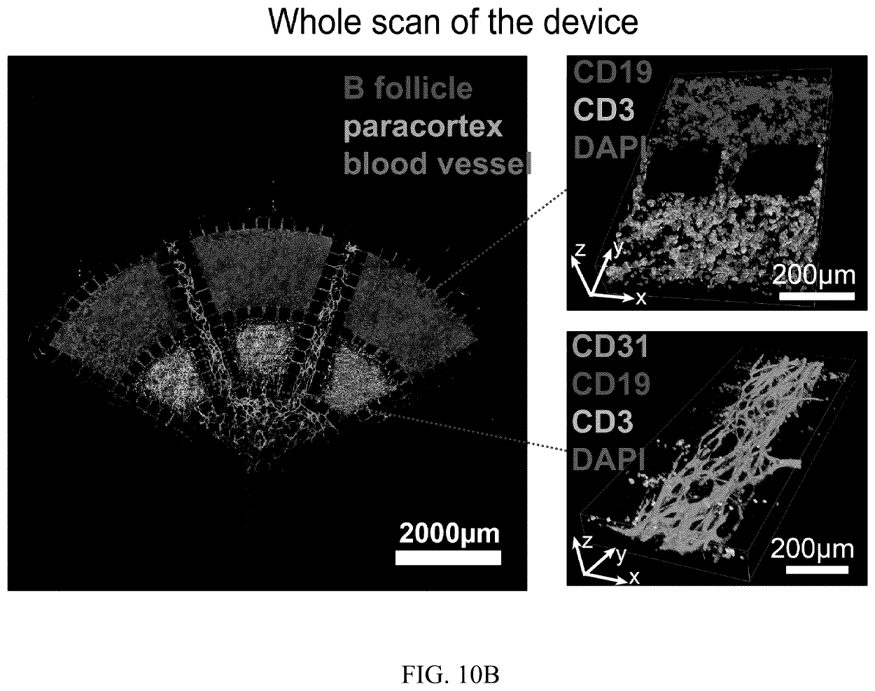

Chip design: The microfluidic LN-on-a-chip model (i.e., LN on a chip device) was constructed with major functional immune niche compartments of LNs (see FIG. 10A & FIG. 10B), including the B cell follicles, paracortex (T cell zone), supported with key stromal niche cells (FRC, FDCs, blood vessels) and biomimetic ECM. Specifically, the paracortex is the T cell region where DCs present antigens to T cells and activate adaptive immune responses [Duckworth B C, Qin R Z, Groom J R, Immunol Rev 2022; 306(1):76-92]. The B cell follicles are areas of B cells within LN that are involved in the production of antibodies and immune responses to specific antigens [Young C, Brink R, Immunity 2021; 54(8):1652-64]. The tissue engineered platform (i.e., LN on a chip device) supports 2-3 weeks of continuous monitoring and real-time imaging, enabling comprehensive, systematic testing of adaptive immune response within a physiologically relevant LN microenvironment. In addition to these cellular compartments, the LN chip was also integrated with surrounding microfluidic channels mimicking lymphatic vessels, subcapsular, and medulla sinus structures that allow continuous perfusion of fluids into the LN niche and study of DC recruitment during adaptive immune response [Elmore S A, Toxicol Pathol 2006; 34(5):425-54].

FIG. 10A and FIG. 10B depict an exemplary Human LN-on-a-Chip in vitro model and imaging scans of the device. FIG. 10A is a diagram depicting an exemplary microfluidic chip design. FIG. 10B are images showing a whole scan of cells on the chip.

Chip microfabrication: The PDMS-based microfluidic chip was fabricated using a standard soft lithography replica-molding method [Ma C et al., Sci Adv 2020; 6(44)]. These different culture compartments were partitioned by regularly spaced PDMS micropillars that confine cell-embedded hydrogels. In some embodiments, T cells, and FRCs were loaded into the paracortex area (T cell zone), and B cells and FDCs were loaded into the B cell follicle region in the LN chip. In addition, 3D blood vessels were created mimicking high endothelial venule (HEV) located in the T cell zone of the LN chip for lymphocytes trafficking, by compartmentalizing human primary endothelial cells (HUVECs) in the paracortex and surrounding channels of the immune compartments. Cell seeding density was optimized for each cell type (10⁶-10⁸ cells/ml) based on physiological data at different locations in the LNs. After forming the LN paracortex, B cell follicles compartments, and 3D blood vessels after 5 days, DCs were loaded from the microfluidic lymphatic vessel channels and entered the paracortex region with perfusion of fluids with a digitally controllable pressure pump, to induce adaptive immune response on chip. The flow rate of lymph fluid through human LNs is highly variable, ranging from a few to tens of microliters per hour. Actual rates vary based on factors like health, node location, and physiological conditions [Zawieja D C, Lymphat Res Biol 2009; 7(2):87-96]. The flow rate of the perfusion system was optimized from 0-120 μL/h to simulate lymph flow within the lymphatic system.

Cell samples: PBMC-derived immune cells were used to build the disclosed LN chip model. Specifically, naïve B cells, T cells, and monocytes were isolated from human PBMCs using magnetic beads Cell Isolation Kits (StemCell Technologies). DCs were differentiated from isolated CD14⁺ monocytes via treating with 400 ng/mL GM-CSF and 250 ng/mL IL-4 in complete RPMI medium for 5-6 days. Additionally, human FRCs (Sciencell) and HUVEC (Lonza) were used as stromal cells and for vascularization of the chip. To generate FDCs, stromal cells from palatine tonsils discarded during routine tonsillectomy were incubated with biotinylated anti-CD35 antibodies and then conjugated with streptavidin nanobeads (Biolegend) using a strong magnet (MojoSort magnet) for separation, as described in [Breeuwsma M, Heesters B A, STAR Protoc 2023; 4(3):102404]. ECM components: The ECM of natural LNs is mainly composed of type I, III and IV collagen, laminin, albumin and fibronectin, etc. [Morgado F N, da Silva A V A, Porrozzi R, Cells 2020; 9(3)]. In order to simulate the ECM of natural LNs, a mixture of Matrigel (rich in collagen and laminin) and fibrin hydrogel (Sigma) was used as the ECM of the disclosed model. Fibrin hydrogel is a cytoplasmic matrix that is widely used in the development of vascularized organs in vitro [Wonski B T, Fisher B, Lam M T, Bioengineering (Basel) 2023; 10(7)]. Culture optimizations: Since multiple types of cells were cultured in the disclosed device, culture media was optimized with a mixture of media for HUVEC (EGM™-2, Lonza), FRC (2301, Sciencells) and lymphocytes (RPMI1640, GIBCO) at different ratios (e.g., 1:1:1, 2:1; 1, or 1:2:1). In some embodiments, chemokines were added to promote vascularization (e.g. VEGF) and germinal center formation (e.g. IL-4, and anti-CD40Ab). In some embodiments, 50% of the culture medium was replaced with fresh medium every 2 days. In addition to optimization of culture media, perfusion was tested if static culture fails to maintain primary cells.

Characterization of the spatial organization of LN niche on chip: the chip was immunostained with CD3, CD19, and CD31 respectively on day 10 to prove successful formation of the LN paracortex (T cell zone), B cell follicles compartment, and 3D blood vessels (HEVs). The localization and density of T cells (CD4⁺ Helper T cells, CD8⁺ cytotoxic T cells) and PDPN⁺ FRCs in the paracortex (T cell zone), CD19⁺ B cells and CD35⁺CD21⁺ FDCs in the B cell follicle region of the LN chip was determined by immunostaining (see FIG. 7A through FIG. 7C).

Study the LN stromal niche on Chip: LN stromal cells, including the HEVs, specialized FRCs and FDCs, form reticular microenvironments to support adaptive immune responses not only by structural components, but also by secretion of soluble factors that guide, retain, and promote immune cells in specific zones of the LN [Rodda L B et al., Immunity 2018; 48(5):1014-28.e6]. Depending on their localization in the LN, these cells display heterogeneous properties supporting the different activities of the adaptive immune response.

3D blood vessels (HEVs) facilitate the recruitment process of T cells and B cells in LN. Based on the disclosed results, 3D blood vessels can be formed through self-assembly and grow into the T cell zone on the chip in 5-7 days. The area and diameter of formed vessels were measured under a confocal microscope (see FIG. 10B), and the intactness of the blood vessels were confirmed by Dextran Alexa Fluor™ 647 (Thermo Fisher). In some embodiments, more CD157⁺ perivascular FRCs surrounding CD31⁺ vessels may be added in the T cell zone support the intactness of the blood vessels [Alvarenga H G, Marti L, J Immunol Res 2014; 2014:402038].

T cell region FRCs (TRCs) form a reticular system to facilitate lymph flow and T cell-DC interactions within the paracortex [Katakai T et al., J Exp Med 2004; 200(6):783-95]. First, it was shown that in response to continuous contact with lymphocytes, FRCs secrete ER-TR7 antigen as an ECM component to form FRC stromal reticulum within 5 days, a reticular meshwork of FRCs and ECM that supports the movement and interactions of DCs and T cells within the paracortex area [Katakai T et al., J Exp Med 2004; 200(6):783-95]. In some embodiments, CD4⁺ T cells and FRCs pre-labeled with CellTracker Red and DID are loaded onto the LN on a chip device, specifically to examine the effect of frequency of T cell-FRC contacts in the T cell region on the production of the ER-TR7 antigen using ELISA, deposition of laminin or fibronectin during the formation of the FRC stromal reticulum using immunostaining.

FDCs are located in B cell follicles, where they present antigen via complement receptors to B cells and form a dense network support primary B-follicles and germinal center formation [Barnett L G et al., J Immunol 2014; 192(8):3607-17]. FDCs were stained with CD35 and CD21, which formed an FDC stromal reticulum in the B follicle region after 10 days. Using immunostaining, it was shown that the formation of FDC stromal reticulum is accompanied by the deposition of collagen I and the attachment of a large number of CD19⁺ B cells. If FDC stromal reticulum formation is not ideal, in some embodiments, TNF-α (10-100 ng/mL) is added to create an inflammatory environment within the LN in a manner that promotes the formation of FDC stromal network [Gillot L et al., Cell Mol Life Sci 2021; 78(16):5987-6002; Li N et al., J Immunol 2007; 178(7):4214-21].

Modeling adaptive immune processes to vaccine in the LN-on-a-Chip model: LNs play a pivotal role in human adaptive immune response that are vital for vaccine efficacy. After vaccination, vaccines drain to the LNs, where DCs recognize and phagocytize vaccine antigens, then activate naïve CD4⁺ T cells in the paracortex and induce naïve B cell activation and differentiation into memory B cells and plasma cells in the B follicles that sustain long-term immune responses (see FIG. 10B). Thus, the LN-on-a-Chip model (i.e., LN on a chip device) simulates the key adaptive immune process in LNs (T cell activation, germinal center formation, and antibody secretion) in response to human seasonal influenza virus vaccine (ATCC BEI).

DC maturation and antigen presentation: After capturing the antigen, immature DCs (imDCs) were shown to transform into mature DCs (mDCs) on chip (see FIG. 6A) and entered the paracortical area with lymph fluid, where they contacted and activated T cells (see FIG. 6C). This antigen presentation process marked the beginning of the adaptive immune response [Fu C et al., Immunol Invest 2022; 51(8):2133-58]. Thus, the disclosed LN on a chip device may be used to assess whether vaccine antigens can induce imDC maturation and trigger adaptive immune response. To this end, human seasonal influenza virus vaccine was used to induce DC maturation on or off chip for 7 days, then flow cytometry was used to analyze the maturation efficiency and phenotype of DCs with a panel of surface markers for imDCs (CD11c, CD1a) and mDCs (CD40, CD80, CD83, and HLA-DR). Upon vaccine antigen stimulation, the expression of mDC markers CD40, CD80, CD83, and HLA-DR increased significantly. In some embodiments, Ovalbimin (OVA I, Sigma Aldrich) is used as model antigen [Karandikar S H et al., JCI Insight 2019; 5(8); Blobner J et al., Neurooncol Adv 2021; 3(1):vdab147] to initiate the adaptive immune process in the LN chip, and it was shown that imDCs transformed into mDCs, and thus resulted in enhanced T cell migration, proliferation and activation (see FIG. 6A through FIG. 6D).

T cell migration and activation stimulated by mDCs and FRCs (T cell Zone): Successful antigen presentation by DCs increase T cell migration in LNs. CCL19 and CCL21 released by mDCs, and FRCs attract T cell migration toward mDCs [Koh W H et al., iScience 2020; 23(8):101427; Veerman K et al., Cell Rep 2019; 26(11):3116-31.e5]. First, the concentration changes of CCL19 and CCL21 were determined in the culture medium by ELISA, after constructing the FRC stromal reticulum, and after loading mDCs respectively. Next to verify the enhanced T cell migration in the paracortex area influenced by mDCs and FRCs, the migration of DCs and CD4⁺ T cells pre-labeled with CellTracker Red and DID dyes were tracked under a fluorescence microscopy for 1-3 days under treatment with human seasonal influenza virus vaccine. DC and T cell migration trajectory and speed in the paracortical area were using Image J. Vaccine-induced mDCs resulted in stronger T cell motility in LN (see FIG. 6B). T cell migration along the FRC stromal reticulum toward antigen-carrying mDCs in paracortex area was also shown on chip. With control experiments it was shown that without mDCs and/or FRC stromal reticulum, T cell motility in paracortex area was significantly hindered. Lastly, it was shown that CD4⁺ T cells were activated after mDCs enter the paracortical area. T cell prelibation rate (see FIG. 6C) was measured using EdU (Sigma Aldrich) and changes in key cytokines related to T cell activation (IL-2, IL-10, INF-γ) were detected using ELISA (see FIG. 6D) over 7 days. Additionally, the ratio of two primary T cell subtypes in LNs, CD4⁺ helper T cells and CD8⁺ cytotoxic T cells were analyzed, upon mDC simulation [Poppema S et al., J Exp Med 1981; 153(1):30-41]. Flow cytometry was used to analyze CD4⁺ T cell subsets (Th1, Th2, Th17, Treg cells) after T cell activation.

B cell differentiation and germinal center formation (B cell follicle zone): A key feature of adaptive immune response is the formation of germinal centers in B cell follicles [Young C, Brink R, Immunity 2021; 54(8):1652-64]. In this process, naïve B cells, with the assistance of regulatory T cells, differentiate into memory B cells capable of producing antigen-specific antibodies to sustain long-term immunity (see FIG. 7A). First, the formation of a large number of germinal center-like structures in the B cell follicles region 10 days after mDCs activate T cells was shown. To this end, GL7-expressing B cells were stained to label germinal center structures, and stained cells with CD83 and CXCR4 to define light and dark areas of the germinal center structure (see FIG. 7B) [Wagar L E et al., Nat Med 2021; 27(1):125-35]. The size and spatial distribution of germinal centers within the B follicle were quantified by Image J. Further, differentiation of B cells into CD27⁺ memory B cells and CD138⁺ plasma cells were verified using immunostaining (see FIG. 7C). Furthermore, the subtypes of B cells in the B follicles were quantified using flow cytometry as per the following panel: naïve B cells (CD27⁻CD38⁻), pre-germinal center (GC) B cells (CD27⁻CD38⁺), GC B cells (CD27⁺CD38⁻), memory B cells (CD27⁺CD38⁻), plasma cells (CD27⁺CD38⁺⁺) [Wagar L E et al., Nat Med 2021; 27(1):125-35]. The secretion levels of cytokines related to B cell activation were measured, such as IL-4, IL-6, and IL-7, in the conditioned medium using ELISA. The proliferation (Ki67) rates of T cells and B cells on chip were quantified to confirm the activation of adaptive immune responses upon the vaccine antigen stimulation.

The impact of FDCs on germinal center formation was explored: CD40L-expressing FDCs form a dense network in the B cell follicles are known to contribute to the formation of germinal centers [Barnett L G et al., J Immunol 2014; 192(8):3607-17]. Thus, B cell differentiation and generation center formation was validated with/without FDCs. It was shown on chip that in the absence of FDCs, B cell differentiation and of germinal center formation was impeded. Due to the scarcity of human-derived FDC, it was proposed to replace FDCs with supplementing CD40L and IL-4 in the culture medium [Tangye S G et al., J Immunol 2002; 169(8):4298-306] to promote the formation of germinal center structures. Moreover, germinal centers only form when both T and B cells successfully interact at the T-B border and migrate within the B-cell follicle containing the FDC network. CD4⁺ helper T cells were stained 10 days after mDCs activate T cells, and it was shown on chip that FDCs secreted the chemokine receptor CXCL13, guided CD4⁺ helper T cells into the T-B border region, and interacted with CD19⁺ B cells thus supporting formation of germinal centers [Kranich J, Krautler N J, Front Immunol 2016; 7:225].

Antibody secretion: Secretion of antibodies is an important sign of the adaptive immune response and the effectiveness of vaccine. Therefore, the secretion levels of total IgG, IgM, and vaccine-specific neutralizing antibodies (Influenza A antibody, Influenza B Antibody, Novus Biologicals) were measured over one week from the chip by ELISA after vaccination. Significant increases in secretion levels of these antibodies upon vaccine stimulation was shown on chip. Moreover, DNA-modifying enzymes are required by B cells to switch from one antibody isotype to another. For instance, Activation-Induced Cytidine Deaminase (AID), are exclusively expressed by B cells residing within lymphoid follicles, but they are not expressed by B cells in peripheral blood [Mechtcheriakova D et al., Cancer Immunol Immunother 2012; 61(9):1591-8]. A significant increase in AID expression on B cells in the disclosed LN chip was achieved 10 days after vaccine stimulation (see FIG. 8).

Gene expression (scRNAseq) analysis: as adaptive immune response proceeds, DC maturation (imDC to mDC) T cell differentiation (cytotoxic, helper, regulatory T cells) and B cell differentiation (memory B cells, plasma cells) occurs in LNs. Thus, cells were retrieved from the LN chips and processed with scRNAseq analysis [Stephenson W et al., Nat Commun 2018; 9(1):791]. Variable cellular states and subsets of immune cells were stratified using the single-cell transcriptome sequencing data, enabling a detailed understanding of cellular heterogeneity.

Multidimensional evaluation of vaccine effectiveness: Based on the aforementioned study, five key functional indices were proposed normalized with a range of 0-1 to comprehensively evaluate vaccine effectiveness. 1. Immune cell phenotypes: DC maturation (CD80, CD83, and HLA-DR), T cell proliferation (ki-67) and differentiation types (CD4⁺ Th cells, CD8⁺ cytotoxic T cells), B cell differentiation types (CD27⁺ memory B cells, CD138⁺ plasma cells); 2. Lymphocyte trafficking: DC recruitment and T cell migration; 3. Proteomic characterization related to immune cell homing (CCL19, CCL21), T cell activation (IFN-γ, IL-10, IL-8, IL-2), T cell migration (e.g. CCL17), and B cell differentiation (IL-4, IL-6); 4. Number and size of germinal centers (GL7⁺); 5. Antibody secretions: total IgG, IgM, and vaccine-specific neutralizing antibody concentrations.

The ultimate goal was to develop a robust and reproducible LN-on-a-chip device for large-scale vaccine efficacy assessment in diverse populations, including different age groups, health statuses, genders, and more. In the disclosed example, the LN chip model was validated with immune cells both from young (age: 28 and 29 years) and old (age: 70 and 72 years) population groups and distinct immune responses were observed (see FIG. 9A through FIG. 9C).

It was shown that older age groups exhibit a less robust response to the vaccine. The disclosed LN on a chip device allows one to apply the platform to various populations, including healthy individuals and those with chronic diseases such as diabetes, men and women, among others. The goal was to create a robust and reproducible LN-on-a-Chip platform for assessing vaccines and diseases in diverse populations. The disclosed LN on a chip device allows one to explore how these different factors such as age, gender, race, health status will reshape adaptive immune processes within LNs and thus the efficacy of vaccines. In some embodiments, the disclosed LN-on-a-chip platform can be utilized to assess different types of vaccines and vaccination strategies. For instance, to improve vaccination strategies for the elderly population, the effectiveness of vaccine adjuvants such as aluminum salts (Strem Chemicals) and MF59 (InvivoGen) can be verified. The disclosed LN-on-a-chip device thus will help to discover optimized vaccination strategies for different population groups by combining vaccine adjuvants.

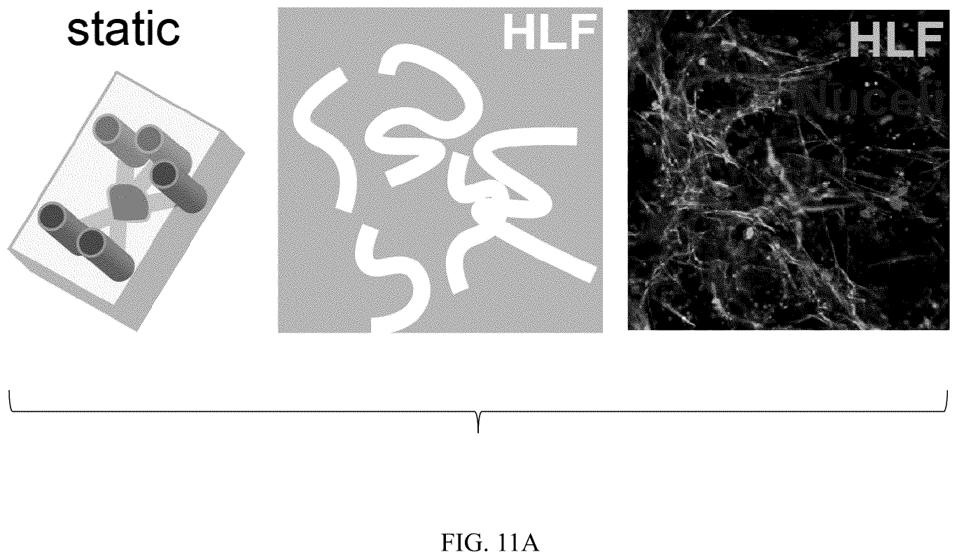

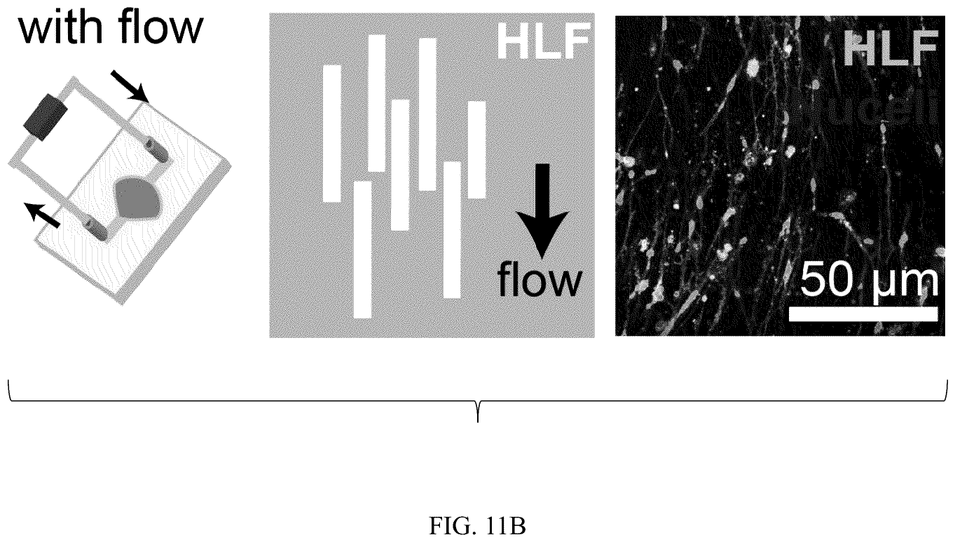

Sustainable perfusion of lymph fluid: Lymph fluid flow is vital for immune stability in LN. Lymph fluid flows in lymphatic vessels can assist in transporting antigens, activating immune cells, establishing chemokine gradients, and maintaining a dynamic environment required for effective immune responses [Birmingham K G et al., iScience 2020; 23(11):101751; Hampton H R, Chtanova T, Front Immunol 2019; 10:1168; Hampton H R et al., Nat Commun 2015; 6:7139]. On one hand, lymph fluid carries antigen-loaded DCs into LN and activates adaptive immune responses; on the other hand, lymph fluid can take away the lactic acid secreted by activated T cells due to enhanced metabolism to maintain pH stability in the LN microenvironment [Wu H et al., Nat Commun 2020; 11(1):4113]. However, current models largely lack precise control of lymph flow in the LN microenvironment. To address this, at least two chip culture modes or configurations may be employed (see FIG. 11A and FIG. 11B): One being a static culture with 5 medium pools (4 mm diameter) to support LN model growth, and another being a dynamic culture using an external peristaltic pump to simulate sustained lymph fluid perfusion. The fluid flow within the LN model was simulated and verified by COMSOL.

FIG. 11A and FIG. 11B depict exemplary configurations for the disclosed LN on a chip device and the related remodeling of stromal cell distribution within the chip through sustained lymph fluid perfusion. FIG. 11A depicts and shows the results for a static culture. FIG. 11B depicts and shows the results for a dynamic culture.

The flow rate of culture medium on the chip was optimized to simulate the flow of lymph fluid in LN. The flow rate of lymph fluid through human LNs is highly variable, ranging from a few microliters per hour to tens of microliters per hour. Actual rates vary based on factors like health, node location, and physiological conditions [Zawieja D C, Lymphat Res Biol 2009; 7(2):87-96]. The flow rate of the perfusion system was optimized from 0-120 μL/h to simulate lymph flow within the lymphatic system. It was observed that a continuously infused lymphatic system (at 60 μL/h) induces a rearrangement process of stromal cells. They (the stromal cells) aligned with the direction of flow, whereas in the stationary cultured LN model, FRC form larger aggregates and are randomly oriented (see FIG. 11A and FIG. 11B). Stromal cell rearrangement was observed in the LN in vitro model under sustainable perfusion of the lymphatic system.

T cell activation efficiency was verified at different flow rates. mDCs that have captured foreign antigens were loaded into the circulating lymphatic system under dynamic culture to compare the efficiency of mDCs activating T cells (proliferation and differentiation) at different flow rates (0-120 μL/h). The antigen presentation process of mDC entering the paracortical area and activating naïve T cells was observed through flow control on chip. Under appropriate lymph flow rate control, the antigen presentation process of DCs was reproduced in the disclosed model. In some embodiments, mDCs and T cells are loaded directly into the paracortex area on the chip and the migration of T cells caused by mDCs can be observed at different flow rates.

In order to further study the role of lymph flow in maintaining the stability of the microenvironment and immune function in LN, the lactate secretion (BioVision, K607) of T cells and the secretion of cytokines (IFN-γ, IL-2, IL-10) were compared related to T cell activation in static culture and dynamic culture. The lactic acid secreted due to the enhanced metabolic capacity during T cell activation reduces the pH of the paracortex area in the LN and inhibits the immune function of T cells [Wu H et al., Nat Commun 2020; 11(1):4113]. Lymph fluid can maintain the pH stability of the microenvironment in LN, and the efficiency of T cell activation was improved under dynamic culture conditions. By adjusting the lymph flow conditions, the DCs-mediated antigen delivery process was reproduced on the chip allowing one to further explore the impact of lymph flow on maintaining the stability of the LN microenvironment.

In addition, in order to further analyze the impact of lymph fluid on adaptive immunity in LN, key information about the adaptive immune response was compared, including changes in lymphocyte subsets (as described in LN Chip Characterizations section), cytokine (IFN-γ, IL-2, IL-4, IL-6, IL-8, IL-10) and antibody secretion (IgG, IgM, and neutralizing antibodies), in static cultures and in cultures with flowing lymph fluid.

### Example 3: Lymph Node-on a Chip

The disclosed innovation describes a 3D organotypic human ‘Lymph node (LN)-on-a-Chip’ model (i.e., LN on a chip device, or “chip”) using state-of-the-art microfluidic organ-on-a-chip technology to create a physiologically relevant LN microenvironment. The disclosed chip mimics natural LN niche compartments (paracortex, follicle, subcapsular sinus) with extracellular matrix (ECM), T cells, B cells, dendritic cells (DCs), fibroblasts, lymphatic vessels, and continuous lymph fluid perfusion. In some embodiments, the disclosed LN-on-a-Chip device is designed for human adaptive immunity modeling and vaccine evaluation. The chip can re-establish adaptive immune environments of varied populations in vitro to monitor crucial immune responses including, but not limited to, cell chemotaxis, lymphocyte activation, and antibody secretion.

Lymph nodes (LNs) play a pivotal role in human adaptive immune responses that are vital for vaccine efficacy. After vaccination, vaccines drain to the LNs, where antigen-presenting cells (APCs) recognize and phagocytize vaccine antigens to induce CD4+ T cell and naïve B cell activation and differentiation that sustain long-term immune responses. The remarkable success of vaccines suggests that this process occurs efficiently and results in long-term immunity against vaccine antigens. However, vaccines are less effective or do not maintain long-term immunity in different populations especially in elderly, due to depletion of CD4+ T lymphocytes, reduction of germinal centers, and fibrosis of the extracellular matrix (ECM) in LNs. Understanding these mechanisms is crucial for developing effective vaccines for different populations. However, current animal models fall short in emulating human adaptive immune responses, urging the need for a human LN model to accurately validate vaccine effectiveness.

The disclosed human LN-on-a-chip device provides a more accurate representation of adaptive immune responses than traditional animal models for vaccine efficacy evaluation. The disclosed technology allows testing of vaccines and adjuvants on long-term adaptive immune responses from various populations, considering factors like age, gender, race, health status, and genetic variations, thus aids in optimizing vaccination strategies for optimal protection for different populations.

Despite the pivotal role of lymph nodes in adaptive immunity, options for lymph node in vitro models are limited. Currently, in vitro lymph node models fall into three categories:

Partial Functional Model of Lymph Node: These models reconstruct some lymph node characteristics. In 2020, Birmingham et al. described a microfluidic device to study subcapsular sinus remodeling under inflammatory conditions, examining physiological architecture and adhesion molecule expression on human monocytes and metastatic cells [Birmingham et al., (2020). iScience, 23(11), 101751]. Additionally, Sonmez et al. developed a microfluidic device to study dendritic cell migration in lymph nodes under gradient chemokine concentrations [Sonmez et al., (2020). Micromachines, 11(4)]. These models simulate partial lymph node features for immune cell chemotaxis and chemokine diffusion studies but lack complete lymph node structure and cell types, limiting their ability to truly reflect the immune microenvironment.

Multicompartment Lymph Node Chip: a previous laboratory devised a microfluidic platform replicating key lymph node features. It divides immune cell regions and simulates the lymph node's spatial microenvironment using 3D hydrogels encapsulating immune cells [Hallfors et al., (2021), Bioengineering (Basel), 8(2); Shanti et al., Front Pharmacol. 2021 Aug. 16; 12:711307]. This platform was employed to assess the impact of drugs like hydroxychloroquine on immune cell movement and reactive oxygen species production. However, it lacks stromal cells and germinal centers, failing to replicate the authentic lymph node niche.

In Vitro Lymph Node Organoids: a previous laboratory generated mouse 3D immune organoids in vitro, capable of regulating B cell developmental dynamics and facilitating germinal center reactions, yielding antigen-specific high-affinity germinal center B cells [Purwada et al., (2019). Biomaterials, 198, 27-36; Purwada & Singh, Nat Protoc. 2017 January; 12(1):168-182; Purwada et al., (2015). Biomaterials, 63, 24-34]. These organoids are currently at the experimental stage in mice, requiring further research to simulate the human lymph node microenvironment in the future.

The disclosed lymph nodes on a chip device comprises a complete lymph node niche, and this platform is of great significance for further studying immune responses in lymph nodes. In some embodiments, blood-derived lymphocytes are utilized to reconstitute LN-on-a-chip. In some embodiments, CD14+ monocytes are induced to generate DCs with GM-CSF, IL-4. LN fibroblasts and HUVEC form stromal cells and blood vessels. In some embodiments, Matrigel serves as ECM. In some embodiments, a microfluidic perfusion system is integrated with the chip to simulate lymphatic fluid within the LNs. In some embodiments, using live-cell imaging, immunofluorescence and ELISA techniques, the lymphocyte phenotype, cytokine and antibody secretions, motility of immune cells (DCs and T cells) and morphology of germinal center from different populations can be visualized, quantified, and compared in vitro.

In some embodiments, the disclosed chip allows one to test the effects of different vaccine (e.g. human seasonal influenza virus vaccine, rabies virus vaccine) and adjuvants (e.g., MF59, AS03) in LNs to analyze Cell-cell communications: DCs and T cells, Tfh and naïve B cells. Immunofluorescence can be used to visualize T cell types (CD4+, CD8+), B cell types (CD27+ memory B cell, CD138+ plasma cell) and germinal centers (GL7+) in vitro. Cytokines: ELISA to track cytokine changes related to T cell activation (IFN-γ, IL-10, IL-2) and B cell differentiation (IL-4, IL-6) across populations. Lymph fluid impact: The chip allows one to compare in vitro LN niche interactions (cell-cell communication, cytokine changes) under fluid vs. static conditions. Antibody secretion analysis: The chip allows one to use ELISA to measure total, IgG, IgM, and vaccine-specific antibodies, assessing vaccine effects across different population groups (age, gender, race etc.).

### Example 4: T Cell Proliferation & B Cell Differentiation on the LN on a Chip Device

T Cell Proliferation: When a T cell receptor (TCR) on the surface of a T cell recognizes and binds to the antigen on a DC, the T cell is activated. At the same time, T cells proliferate, clonally expanding to increase the number of cells specific to the antigen. This proliferation is essential for mounting an effective immune response, allowing for the elimination of pathogens or infected cells and the formation of memory T cells for faster response upon future exposures.

Treatments: To detect T cell proliferation after vaccination on chip, the EdU Cell Proliferation Kit (Sigma Aldrich) and APC anti-human CD3 antibody (Biolegend) was used to perform in situ on the chip on day 7 after introducing the vaccine antigen to stimulate adaptive immunity within the chip. Chips without vaccine antigens served as control.

Measured Changes: the expression of EdU in T cells in the vaccine antigen stimulation group and the control group were observed through confocal microscopy (Nikon Ci2). Through Image J analysis, it was determined that the T cell proliferation in the vaccine antigen group was significantly increased (as shown in FIG. 6C).

B cell differentiation: B cell differentiation in adaptive immunity is a fundamental process through which B cells transform into effector cells capable of producing antibodies against specific antigens, thus playing a crucial role in the humoral immune response. During this process, naïve B cells, with the assistance of regulatory T cells, differentiate into plasma cells capable of producing antigen-specific antibodies and memory B cells that maintain long-term immunity.

Treatments: To detect post-vaccination B cell differentiation on chip, immunofluorescence staining using CD3, CD19, and CD138 antibodies (Biolegend) was performed on day 10 after introduction of the vaccine antigen within the chip. The chip without vaccine antigen served as a control.

Measured Changes: It was observed through confocal microscopy (Nikon Ci2) that the B cells in the vaccine-stimulated group grew in clusters, while the B cells in the control group were evenly dispersed in the chip. At the same time, compared with the control group, the content of CD138+ plasma cells in the vaccine stimulation group was significantly increased (as shown in FIG. 7C).

The following publications are incorporated by reference in their entirety:
B. C. Buckland, The process development challenge for a new vaccine. Nature Medicine 11, S16-S19 (2005).
B. Pulendran, M. M. Davis, The science and medicine of human immunology. Science (New York, N.Y.) 369, (2020).
A. C. Walls et al., Distinct sensitivities to SARS-CoV-2 variants in vaccinated humans and mice. Cell Rep 40, 111299 (2022).
D. Calina et al., COVID-19 vaccines: ethical framework concerning human challenge studies. Daru: Journal of Faculty of Pharmacy, Tehran University of Medical Sciences 28, 807-812 (2020).
E. Moysi, R. M. Paris, R. Le Grand, R. A. Koup, C. Petrovas, Human lymph node immune dynamics as driver of vaccine efficacy: an understudied aspect of immune responses. Expert Rev Vaccines 21, 633-644 (2022).
I. J. Amanna, M. K. Slifka, Successful Vaccines. Curr Top Microbiol Immunol 428, 1-30 (2020).
M. T. Witkowski et al., Extensive Remodeling of the Immune Microenvironment in B Cell Acute Lymphoblastic Leukemia. Cancer Cell 37, 867-882 e812 (2020).
Z. Zhang et al., An in silico glioblastoma microenvironment model dissects the immunological mechanisms of resistance to PD-1 checkpoint blockade immunotherapy. Small Methods 5, (2021).
B. Ma et al., A digital nanoplasmonic microarray immunosensor for multiplexed cytokine monitoring during CAR T-cell therapy from a leukemia tumor microenvironment model. Biosensors & Bioelectronics 230, 115247 (2023).
X. Cui et al., Dissecting the immunosuppressive tumor microenvironments in Glioblastoma-on-a-Chip for optimized PD-1 immunotherapy. Elife 9, (2020).
C. Ma et al., Leukemia-on-a-chip: Dissecting the chemoresistance mechanisms in B cell acute lymphoblastic leukemia bone marrow niche. Science Advances 6, (2020).
M. Cakala-Jakimowicz, P. Kolodziej-Wojnar, M. Puzianowska-Kuznicka, Aging-Related Cellular, Structural and Functional Changes in the Lymph Nodes: A Significant Component of Immunosenescence? An Overview. Cells 10, (2021).
J. Chen, J. C. Deng, D. R. Goldstein, How aging impacts vaccine efficacy: known molecular and cellular mechanisms and future directions. Trends Mol Med 28, 1100-1111 (2022).
E. Dube, J. K. Ward, P. Verger, N. E. MacDonald, Vaccine Hesitancy, Acceptance, and Anti-Vaccination: Trends and Future Prospects for Public Health. Annu Rev Public Health 42, 175-191 (2021).
M. Jeyanathan et al., Immunological considerations for COVID-19 vaccine strategies. Nat Rev Immunol 20, 615-632 (2020).
D. Y. Cassandra Pingali, Laurie D. Elam-Evans, Lauri E. Markowitz, Charnetta L. Williams, Benjamin Fredua, Lucy A. McNamara, Shannon Stokley, and James A. Singleton, National, Regional, State, and Selected Local Area Vaccination Coverage Among Adolescents Aged 13-17 Years—United States, 2020. Morbidity and Mortality Weekly Report 70, 1183-1190 (2021).
S. Sell, How vaccines work: immune effector mechanisms and designer vaccines. Expert Rev Vaccines 18, 993-1015 (2019).
L. Sompayrac, How the Immune System Works. (2019).
Y. Ding, Z. Li, A. Jaklenec, Q. Hu, Vaccine delivery systems toward lymph nodes. Adv Drug Deliv Rev 179, 113914 (2021).
J. Leleux, A. Atalis, K. Roy, Engineering immunity: Modulating dendritic cell subsets and lymph node response to direct immune-polarization and vaccine efficacy. J Control Release 219, 610-621 (2015).
S. M. Grant, M. Lou, L. Yao, R. N. Germain, A. J. Radtke, The lymph node at a glance—how spatial organization optimizes the immune response. J Cell Sci 133, (2020).
O. Ahmadi, J. L. McCall, M. D. Stringer, Does senescence affect lymph node number and morphology?A systematic review. ANZ J Surg 83, 612-618 (2013).
B. E. Burrell et al., Tolerance and lymphoid organ structure and function. Front Immunol 2, 64 (2011).
S. Liao, P. Y. von der Weid, Lymphatic system: an active pathway for immune protection. Semin Cell Dev Biol 38, 83-89 (2015).
J. E. Chang, S. J. Turley, Stromal infrastructure of the lymph node and coordination of immunity. Trends Immunol 36, 30-39 (2015).
Y. Chen, S. De Koker, B. G. De Geest, Engineering Strategies for Lymph Node Targeted Immune Activation. Acc Chem Res 53, 2055-2067 (2020).
T. Ozulumba, A. N. Montalbine, J. E. Ortiz-Cardenas, R. R. Pompano, New tools for immunologists: models of lymph node function from cells to tissues. Front Immunol 14, 1183286 (2023).
T. T. Murooka, T. R. Mempel, Intravital microscopy in BLT-humanized mice to study cellular dynamics in HIV infection. J Infect Dis 208 Suppl 2, S137-144 (2013).
C. Zhang et al., Humanized Mice for Studies of HIV-1 Persistence and Elimination. Pathogens 12, (2023).
R. S. Herati, E. J. Wherry, What Is the Predictive Value of Animal Models for Vaccine Efficacy in Humans? Consideration of Strategies to Improve the Value of Animal Models. Cold Spring Harb Perspect Biol 10, (2018).
A. Purwada et al., Ex vivo engineered immune organoids for controlled germinal center reactions. Biomaterials 63, 24-34 (2015).
K. G. Birmingham et al., Lymph Node Subcapsular Sinus Microenvironment-On-A-Chip Modeling Shear Flow Relevant to Lymphatic Metastasis and Immune Cell Homing. iScience 23, 101751 (2020).
N. Hallfors et al., Multi-Compartment Lymph-Node-on-a-Chip Enables Measurement of Immune Cell Motility in Response to Drugs. Bioengineering (Basel) 8, (2021).
P. Moura Rosa, N. Gopalakrishnan, H. Ibrahim, M. Haug, O. Halaas, The intercell dynamics of T cells and dendritic cells in a lymph node-on-a-chip flow device. Lab Chip 16, 3728-3740 (2016).
C. Ma, Y. Peng, H. Li, W. Chen, Organ-on-a-Chip: A New Paradigm for Drug Development. Trends Pharmacol Sci 42, 119-133 (2021).
S. Jalkanen, M. Salmi, Lymphatic endothelial cells of the lymph node. Nat Rev Immunol 20, 566-578 (2020).
J. Lian, A. D. Luster, Chemokine-guided cell positioning in the lymph node orchestrates the generation of adaptive immune responses. Curr Opin Cell Biol 36, 1-6 (2015).
B. C. Duckworth, R. Z. Qin, J. R. Groom, Spatial determinates of effector and memory CD8(+) T cell fates. Immunol Rev 306, 76-92 (2022).
C. Young, R. Brink, The unique biology of germinal center B cells. Immunity 54, 1652-1664 (2021).
S. A. Elmore, Histopathology of the lymph nodes. Toxicol Pathol 34, 425-454 (2006).
D. C. Zawieja, Contractile physiology of lymphatics. Lymphat Res Biol 7, 87-96 (2009).
R. Caeser et al., Genetic modification of primary human B cells to model high-grade lymphoma. Nat Commun 10, 4543 (2019).
F. N. Morgado, A. V. A. da Silva, R. Porrozzi, Infectious Diseases and the Lymphoid Extracellular Matrix Remodeling: A Focus on Conduit System. Cells 9, (2020).
B. T. Wonski, B. Fisher, M. T. Lam, Hydrogel Coating Optimization to Augment Engineered Soft Tissue Mechanics in Tissue-Engineered Blood Vessels. Bioengineering (Basel) 10, (2023).
L. B. Rodda et al., Single-Cell RNA Sequencing of Lymph Node Stromal Cells Reveals Niche-Associated Heterogeneity. Immunity 48, 1014-1028.e1016 (2018).
H. G. Alvarenga, L. Marti, Multifunctional roles of reticular fibroblastic cells: more than meets the eye? J Immunol Res 2014, 402038 (2014).
T. Katakai, T. Hara, M. Sugai, H. Gonda, A. Shimizu, Lymph node fibroblastic reticular cells construct the stromal reticulum via contact with lymphocytes. J Exp Med 200, 783-795 (2004).
W. H. Koh et al., HIV-Captured DCs Regulate T Cell Migration and Cell-Cell Contact Dynamics to Enhance Viral Spread. iScience 23, 101427 (2020).
K. Veerman, C. Tardiveau, F. Martins, J. Coudert, J. P. Girard, Single-Cell Analysis Reveals Heterogeneity of High Endothelial Venules and Different Regulation of Genes Controlling Lymphocyte Entry to Lymph Nodes. Cell Rep 26, 3116-3131.e3115 (2019).
L. G. Barnett et al., B cell antigen presentation in the initiation of follicular helper T cell and germinal center differentiation. J Immunol 192, 3607-3617 (2014).
N. Li, K. Boyd, P. J. Dempsey, D. A. Vignali, Non-cell autonomous expression of TNF-alpha-converting enzyme ADAM17 is required for normal lymphocyte development. J Immunol 178, 4214-4221 (2007).
L. Gillot, L. Baudin, L. Rouaud, F. Kridelka, A. Noel, The pre-metastatic niche in lymph nodes: formation and characteristics. Cell Mol Life Sci 78, 5987-6002 (2021).
H. R. Hampton, T. Chtanova, Lymphatic Migration of Immune Cells. Front Immunol 10, 1168 (2019).
H. R. Hampton, J. Bailey, M. Tomura, R. Brink, T. Chtanova, Microbe-dependent lymphatic migration of neutrophils modulates lymphocyte proliferation in lymph nodes. Nat Commun 6, 7139 (2015).
H. Wu et al., T-cells produce acidic niches in lymph nodes to suppress their own effector functions. Nat Commun 11, 4113 (2020).
C. Fu, T. Ma, L. Zhou, Q.-S. Mi, A. Jiang, Dendritic Cell-Based Vaccines Against Cancer: Challenges, Advances and Future Opportunities. Immunological Investigations 51, 2133-2158 (2022).
S. H. Karandikar et al., New epitopes in ovalbumin provide insights for cancer neoepitopes. JCI Insight 5, (2019).
J. Blobner et al., Comparative evaluation of T-cell receptors in experimental glioma-draining lymph nodes. Neuro-oncology Advances 3, vdab147 (2021).
S. Poppema, A. K. Bhan, E. L. Reinherz, R. T. McCluskey, S. F. Schlossman, Distribution of T cell subsets in human lymph nodes. J Exp Med 153, 30-41 (1981).
L. E. Wagar et al., Modeling human adaptive immune responses with tonsil organoids. Nat Med 27, 125-135 (2021).
S. G. Tangye, A. Ferguson, D. T. Avery, C. S. Ma, P. D. Hodgkin, Isotype switching by human B cells is division-associated and regulated by cytokines. J Immunol 169, 4298-4306 (2002).
J. Kranich, N. J. Krautler, How Follicular Dendritic Cells Shape the B-Cell Antigenome. Front Immunol 7, 225 (2016).
D. Mechtcheriakova, M. Svoboda, A. Meshcheryakova, E. Jensen-Jarolim, Activation-induced cytidine deaminase (AID) linking immunity, chronic inflammation, and cancer. Cancer Immunology, Immunotherapy: CII 61, 1591-1598 (2012).
W. Stephenson et al., Single-cell RNA-seq of rheumatoid arthritis synovial tissue using low-cost microfluidic instrumentation. Nat Commun 9, 791 (2018).
T. Zhao et al., Vaccine adjuvants: mechanisms and platforms. Signal Transduction and Targeted Therapy 8, 283 (2023).
E. M. B. Scholz, A. D. M. Kashuba, The Lymph Node Reservoir: Physiology, HIV Infection, and Antiretroviral Therapy. Clin Pharmacol Ther 109, 918-927 (2021).
Jeyanathan M, Afkhami S, Smaill F, Miller M S, Lichty B D, Xing Z. Immunological considerations for COVID-19 vaccine strategies. Nat Rev Immunol. 2020; 20(10):615-32.
Gillot L, Baudin L, Rouaud L, Kridelka F, Noel A. The pre-metastatic niche in lymph nodes: formation and characteristics. Cell Mol Life Sci. 2021; 78(16):5987-6002.
Buckland B C. The process development challenge for a new vaccine. Nat Med. 2005; 11(4 Suppl):S16-S9.
Pulendran B, Davis M M. The science and medicine of human immunology. Science. 2020; 369(6511).
Walls A C, VanBlargan L A, Wu K, Choi A, Navarro M J, Lee D, et al. Distinct sensitivities to SARS-CoV-2 variants in vaccinated humans and mice. Cell Rep. 2022; 40(9):111299.
Calina D, Hartung T, Docea A O, Spandidos D A, Egorov A M, Shtilman M I, et al. COVID-19 vaccines: ethical framework concerning human challenge studies. Daru. 2020; 28(2):807-12.
Leleux J, Atalis A, Roy K. Engineering immunity: Modulating dendritic cell subsets and lymph node response to direct immune-polarization and vaccine efficacy. J Control Release. 2015; 219:610-21.
Grant S M, Lou M, Yao L, Germain R N, Radtke A J. The lymph node at a glance—how spatial organization optimizes the immune response. J Cell Sci. 2020; 133(5).
Cakala-Jakimowicz M, Kolodziej-Wojnar P, Puzianowska-Kuznicka M. Aging-Related Cellular, Structural and Functional Changes in the Lymph Nodes: A Significant Component of Immunosenescence? An Overview. Cells. 2021; 10(11).
Ahmadi O, McCall J L, Stringer M D. Does senescence affect lymph node number and morphology?A systematic review. ANZ J Surg. 2013; 83(9):612-8.
Chen J, Deng J C, Goldstein D R. How aging impacts vaccine efficacy: known molecular and cellular mechanisms and future directions. Trends Mol Med. 2022; 28(12):1100-11.
Liao S, von der Weid P Y. Lymphatic system: an active pathway for immune protection. Semin Cell Dev Biol. 2015; 38:83-9.
Fletcher A L, Acton S E, Knoblich K. Lymph node fibroblastic reticular cells in health and disease. Nat Rev Immunol. 2015; 15(6):350-61.
Ozulumba T, Montalbine A N, Ortiz-Cardenas J E, Pompano R R. New tools for immunologists: models of lymph node function from cells to tissues. Front Immunol. 2023; 14:1183286.
Herati R S, Wherry E J. What Is the Predictive Value of Animal Models for Vaccine Efficacy in Humans? Consideration of Strategies to Improve the Value of Animal Models. Cold Spring Harb Perspect Biol. 2018; 10(4).
Purwada A, Jaiswal M K, Ahn H, Nojima T, Kitamura D, Gaharwar A K, et al. Ex vivo engineered immune organoids for controlled germinal center reactions. Biomaterials. 2015; 63:24-34.
Kwiatkowska K M, Mkindi C G, Nielsen C M. Human lymphoid tissue sampling for vaccinology. Front Immunol. 2022; 13:1045529.
Stern P L. Key steps in vaccine development. Ann Allergy Asthma Immunol. 2020; 125(1):17-27.
Sonmez U M, Wood A, Justus K, Jiang W, Syed-Picard F, LeDuc P R, et al. Chemotactic Responses of Jurkat Cells in Microfluidic Flow-Free Gradient Chambers. Micromachines. 2020; 11(4).
Chen X Y, Butt A M, Mohd Amin M C I. Enhanced paracellular delivery of vaccine by hydrogel microparticles-mediated reversible tight junction opening for effective oral immunization. Journal of Controlled Release: Official Journal of the Controlled Release Society. 2019; 311-312:50-64.
Goyal G, Prabhala P, Mahajan G, Bausk B, Gilboa T, Xie L, et al. Ectopic Lymphoid Follicle Formation and Human Seasonal Influenza Vaccination Responses Recapitulated in an Organ-on-a-Chip. Adv Sci (Weinh). 2022; 9(14):e2103241.
Westdorp H, Creemers J H A, van Oort I M, Schreibelt G, Gorris M A J, Mehra N, et al. Blood-derived dendritic cell vaccinations induce immune responses that correlate with clinical outcome in patients with chemo-naïve castration-resistant prostate cancer. J Immunother Cancer. 2019; 7(1):302.
Cabrita R, Lauss M, Sanna A, Donia M, Skaarup Larsen M, Mitra S, et al. Tertiary lymphoid structures improve immunotherapy and survival in melanoma. Nature. 2020; 577(7791):561-5.
Römer P S, Berr S, Avota E, Na S-Y, Battaglia M, ten Berge I, et al. Preculture of PBMCs at high cell density increases sensitivity of T-cell responses, revealing cytokine release by CD28 superagonist TGN1412. Blood. 2011; 118(26):6772-82.
Duckworth B C, Qin R Z, Groom J R. Spatial determinates of effector and memory CD8(+) T cell fates. Immunol Rev. 2022; 306(1):76-92.
Young C, Brink R. The unique biology of germinal center B cells. Immunity. 2021; 54(8):1652-64.
Elmore S A. Histopathology of the lymph nodes. Toxicol Pathol. 2006; 34(5):425-54.
Ma C, Witkowski M T, Harris J, Dolgalev I, Sreeram S, Qian W, et al. Leukemia-on-a-chip: Dissecting the chemoresistance mechanisms in B cell acute lymphoblastic leukemia bone marrow niche. Sci Adv. 2020; 6(44).
Zawieja D C. Contractile physiology of lymphatics. Lymphat Res Biol. 2009; 7(2):87-96.
Breeuwsma M, Heesters B A. Protocol for the isolation and purification of human follicular dendritic cells for functional assays. STAR Protoc. 2023; 4(3):102404.
Morgado F N, da Silva A V A, Porrozzi R. Infectious Diseases and the Lymphoid Extracellular Matrix Remodeling: A Focus on Conduit System. Cells. 2020; 9(3).
Wonski B T, Fisher B, Lam M T. Hydrogel Coating Optimization to Augment Engineered Soft Tissue Mechanics in Tissue-Engineered Blood Vessels. Bioengineering (Basel). 2023; 10(7).
Rodda L B, Lu E, Bennett M L, Sokol C L, Wang X, Luther S A, et al. Single-Cell RNA Sequencing of Lymph Node Stromal Cells Reveals Niche-Associated Heterogeneity. Immunity. 2018; 48(5):1014-28.e6.
Alvarenga H G, Marti L. Multifunctional roles of reticular fibroblastic cells: more than meets the eye? J Immunol Res. 2014; 2014:402038.
Katakai T, Hara T, Sugai M, Gonda H, Shimizu A. Lymph node fibroblastic reticular cells construct the stromal reticulum via contact with lymphocytes. J Exp Med. 2004; 200(6):783-95.
Barnett L G, Simkins H M, Barnett B E, Kom L L, Johnson A L, Wherry E J, et al. B cell antigen presentation in the initiation of follicular helper T cell and germinal center differentiation. J Immunol. 2014; 192(8):3607-17.
Li N, Boyd K, Dempsey P J, Vignali D A. Non-cell autonomous expression of TNF-alpha-converting enzyme ADAM17 is required for normal lymphocyte development. J Immunol. 2007; 178(7):4214-21.
Fu C, Ma T, Zhou L, Mi Q-S, Jiang A. Dendritic Cell-Based Vaccines Against Cancer: Challenges, Advances and Future Opportunities. Immunol Invest. 2022; 51(8):2133-58.
Karandikar S H, Sidney J, Sette A, Selby M J, Korman A J, Srivastava P K. New epitopes in ovalbumin provide insights for cancer neoepitopes. JCI Insight. 2019; 5(8).
Blobner J, Kilian M, Tan C L, Aslan K, Sanghvi K, Meyer J, et al. Comparative evaluation of T-cell receptors in experimental glioma-draining lymph nodes. Neurooncol Adv. 2021; 3(1):vdab147.
Koh W H, Lopez P, Ajibola O, Parvarchian R, Mohammad U, Hnatiuk R, et al. HIV-Captured DCs Regulate T Cell Migration and Cell-Cell Contact Dynamics to Enhance Viral Spread. iScience. 2020; 23(8):101427.
Veerman K, Tardiveau C, Martins F, Coudert J, Girard J P. Single-Cell Analysis Reveals Heterogeneity of High Endothelial Venules and Different Regulation of Genes Controlling Lymphocyte Entry to Lymph Nodes. Cell Rep. 2019; 26(11):3116-31.e5.
Poppema S, Bhan A K, Reinherz E L, McCluskey R T, Schlossman S F. Distribution of T cell subsets in human lymph nodes. J Exp Med. 1981; 153(1):30-41.
Wagar L E, Salahudeen A, Constantz C M, Wendel B S, Lyons M M, Mallajosyula V, et al. Modeling human adaptive immune responses with tonsil organoids. Nat Med. 2021; 27(1):125-35.
Tangye S G, Ferguson A, Avery D T, Ma C S, Hodgkin P D. Isotype switching by human B cells is division-associated and regulated by cytokines. J Immunol. 2002; 169(8):4298-306.
Kranich J, Krautler N J. How Follicular Dendritic Cells Shape the B-Cell Antigenome. Front Immunol. 2016; 7:225.
Mechtcheriakova D, Svoboda M, Meshcheryakova A, Jensen-Jarolim E. Activation-induced cytidine deaminase (AID) linking immunity, chronic inflammation, and cancer. Cancer Immunol Immunother. 2012; 61(9):1591-8.
Stephenson W, Donlin L T, Butler A, Rozo C, Bracken B, Rashidfarrokhi A, et al. Single-cell RNA-seq of rheumatoid arthritis synovial tissue using low-cost microfluidic instrumentation. Nat Commun. 2018; 9(1):791.
Ager A, May M J. Understanding high endothelial venules: Lessons for cancer immunology. Oncoimmunology. 2015; 4(6):e1008791.
Petrova T V, Koh G Y. Biological functions of lymphatic vessels. Science. 2020; 369(6500).
Birmingham K G, O'Melia M J, Bordy S, Reyes Aguilar D, El-Reyas B, Lesinski G, et al. Lymph Node Subcapsular Sinus Microenvironment-On-A-Chip Modeling Shear Flow Relevant to Lymphatic Metastasis and Immune Cell Homing. iScience. 2020; 23(11):101751.
de Castro Pinho J, Förster R. Lymph-Derived Neutrophils Primarily Locate to the Subcapsular and Medullary Sinuses in Resting and Inflamed Lymph Nodes. Cells. 2021; 10(6).
Hampton H R, Chtanova T. Lymphatic Migration of Immune Cells. Front Immunol. 2019; 10:1168.
Hampton H R, Bailey J, Tomura M, Brink R, Chtanova T. Microbe-dependent lymphatic migration of neutrophils modulates lymphocyte proliferation in lymph nodes. Nat Commun. 2015; 6:7139.
Wu H, Estrella V, Beatty M, Abrahams D, El-Kenawi A, Russell S, et al. T-cells produce acidic niches in lymph nodes to suppress their own effector functions. Nat Commun. 2020; 11(1):4113.
Pompano et al., “3D spatially patterned lymph node on a microfluidic device”, US Patent Publication No.: US 2021/0054324 A1
GOYAL et al., “Human lymphoid tissue-on-chip”, US Patent Publication No.: US 2022/0250055 A1
Stark et al., “Antibody producing microfluidic devices”, US Patent Publication No.: US 2022/0282211 A1

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A lymph node on a chip device, comprising:
a microfluidic chip comprising a top and bottom surface;
a central chamber embedded in the microfluidic chip;
one or more openings in the chip fluidly connected to the central chamber with one or more channels;
a plurality of micropillars arranged within the central chamber such that the central chamber is partitioned into an inner region, one or more outer regions positioned around the inner region, and a circumferential region surrounding the one or more outer regions, with the micropillars forming one or more channels extending from the inner region to at least the outer region; and
one or more paracortex cells configured to mimic a paracortex region positioned in the inner region, one or more follicle cells configured to mimic one or more follicle regions positioned in the one or more outer regions, and one or more interfollicular cells configured to mimic one or more interfollicular regions positioned in the one or more channels.

2. The device of claim 1, wherein the one or more paracortex cells are selected from the group consisting of: paracortex niche cells, paracortex niche supporting cells, stromal cells, T cells, T lymphocytes, T helper cells, cytotoxic T cells, regulatory T cells (Tregs), dendritic cells (DC), fibroblastic reticular cells (FRC), and blood vessel endothelial cells.

3. The device of claim 2, wherein the one or more follicle cells are selected from the group consisting of: follicle niche cells, follicle niche supporting cells, B cells, naïve B cells, memory B cells, plasma cells, follicular dendritic cells (FDC), and blood-derived dendritic cells (DC).

4. The device of claim 3, wherein the one or more interfollicular cells are selected from the group consisting of: interfollicular niche cells, interfollicular niche supporting cells, stromal cells, fibroblastic reticular cells (FRC), endothelial cells, blood vessel cells, blood vessel endothelial cells, T cells, T lymphocytes, T helper cells, cytotoxic T cells, and regulatory T cells (Tregs)

5. The device of claim 4, further comprising one or more subcapsular sinus cells configured to mimic a subcapsular sinus positioned in the circumferential region.

6. The device of claim 5, wherein the one or more subcapsular sinus cells are selected from the group consisting of: sinus cells, subcapsular sinus cells, macrophages, lymphatic endothelial cells, marginal reticular cells, dendritic cells.

7. The device of claim 6, further comprising one or more cell culture media components in the central chamber selected from the group consisting of: cell culture media, growth factors, growth factors for fibroblasts, growth factors for endothelial cells, fibroblast medium (2301, ScienCell), RPMI 1640 medium (Gibco), endothelial cell growth medium (EGM-2, Lonza), and Vascular endothelial growth factor (VEGF).

8. The device of claim 7, further comprising one or more extracellular matrix components in the central chamber selected from the group consisting of: membrane, basement membrane, solubilized basement membrane, Matrigel (Corning), polymer, gel, hydrogel, fibrin hydrogel (Sigma), collagen, collagen I (Corning).

9. The device of claim 8, further comprising one or more reservoirs embedded in the microfluidic chip fluidly connected to the central chamber.

10. The device of claim 9, wherein the central chamber is at least partially formed in one or more shapes selected from the group consisting of: irregular, limagon, cardioid, heart, kidney, elliptical, ovular, and round.

11. The device of claim 10, wherein the central chamber is formed in a cardioid shape and the one or more reservoirs comprise a first reservoir fluidly connected to the cusp region of the central chamber, and a second and third reservoirs fluidly connected to positions opposite the cusp region of the central chamber.

12. The device of claim 11, wherein each micropillar of the plurality of micropillars is at least partially formed in one or more shapes selected from the group consisting of: column, cylinder, round, frustum, cone, oblong, irregular.

13. The device of claim 12, wherein each micropillar has a width or diameter, a height, and a spacing to the next micropillar; wherein the width or diameter ranges between about 100 μm and about 200 μm, the height ranges between about 50 μm and about 200 μm, and the spacing to the next micropillar ranges between about 50 μm and about 200 μm.

14. A method of measuring an immune response, comprising the steps of:
providing the device of claim 1;
administering at least one treatment to the device; and
determining treatment responsiveness based on at least one measured change on the device.

15. The method of claim 14, wherein the at least one treatment is selected from the group consisting of: vaccine, mRNA vaccine, inactivated vaccine, adenovirus-based vaccine, small molecule, protein, and nucleic acid molecule.

16. The method of claim 15, wherein the measured change comprises any of: antibody secretions, cell migration, cell proliferation, cell activation, cell infiltration, cell speed, cell trajectory, cell distance, cell position, cell motility, cell maturation, cell maturation efficiency, cell maturation efficiency, cell phenotype, cell differentiation, cell concentration, cell recruitment, cell migration within one region (e.g., the inner region), cell migration from one region to another (e.g., the inner region to the interfollicular region), pH in the central chamber, pH in a culture medium, number, size and spatial distribution of germinal centers, number, size and spatial distribution of germinal centers in the one or more outer regions.

17. The method of claim 16, wherein the at least one measured change comprises: antibody secretion (e.g., immunoglobulin such as IgG, IgM, IgE, IgD, or secretion levels of total IgG, secretion levels of total IgM), and T cell migration from the paracortex region to the interfollicular region.

18. The method of claim 17, wherein the at least one measured change further comprises cytokine concentration.

19. The method of claim 18, further comprising the step of administering at least one adjuvant in combination with the at least one treatment.

20. The method of claim 19, wherein the at least one adjuvant is selected from the group consisting of: AS03, CpG Oligodeoxynucleotides, squalene, monophosphoryl Lipid A, aluminum salts and MF59.
